(19) United States
(12) Patent Application Publication
(10) Pub. No.: US 2023/0394989 A1
(43) Pub. Date: Dec. 7, 2023

(54) MULTI-SENSORY BASED PERFORMANCE ENHANCEMENT SYSTEM

(71) Applicant: SPEADTECH LIMITED, Hong Kong SAR (HK)

(72) Inventors: Peter Hill, Hong Kong (HK); Robert Louis Wilson, Brentford (GB)

(21) Appl. No.: 18/250,193

(22) PCT Filed: Apr. 21, 2021
(86) PCT No.: PCT/US2021/028476

§ 371 Date: Apr. 21, 2023

Related U.S. Application Data
(60) Provisional application No. 63/014,034, filed on Apr. 22, 2020.

(51) Int. Cl. G09B 19/16; G06F 3/16; G07C 5/02; G09B 5/04

(52) U.S. Cl. CPC G09B 19/167; G06F 3/16; G07C 5/02; G09B 5/04; A61B 5/6893

(57) ABSTRACT

A system is disclosed for communicating audio messages about a vehicle or equipment to a user or other person to improve performance. The vehicle could be a racecar and the user could be a racecar driver. The system can include a speaker to convey audio messages to the user, including real time messages for helping the user assess and improve physical performance. The messages may be generated based on various types of data, including sensor data. Sensor data may be collected by vehicle sensors of a vehicle or by systems external to the vehicle.

### CROSS-REFERENCE TO RELATED APPLICATIONS

This application is the U.S. National Phase under 35 U.S.C. § 371 of International Application PCT/US2021/028476, filed Apr. 21, 2021, which claims priority to U.S. Application No. 63/014,034, filed Apr. 22, 2020, the entire contents of each of which are incorporated by reference herein and made a part of this specification.

### BACKGROUND

Racecar drivers and other athletes commonly rely on sensor data to evaluate and improve their performance. Typically, however, the relevant sensor data and reports are provided to the athlete after the performance is complete. For example, a racecar driver may, as part of a training session, complete several laps on a track and then stop to review a report that is based on collected sensor data.

### SUMMARY

The present disclosure can provide for the creation of an audible language for vehicle, machine, or equipment dynamics to give the driver, athlete, or other person feedback for improving vehicle, machine, or equipment control. The audible language is comprised of sophisticated sound metaphors. The sound metaphors can be used in combination with other sensory-based messages, such as haptic and/or vibration metaphors. These metaphors communicate vehicle dynamics as cue messages, in real time, or post event as an audible or other expression. How the language is provided conveys certain desired details of operation. Vehicle data can be turned into music, with appropriate beats and notes. The driver or operator can adjust inputs (steering, braking, accelerating, regenerative braking, energy harvesting, deployment of energy, etc.) to stay “on tune”. Flat or sharp notes can indicate the optimum or desired thresholds have been exceeded or not reached. Rhythms or beats can be used to create a low cognitive load warning or impending event. Rhythm or beats can indicate what inputs to adjust at the vehicle or equipment controls, in anticipation of the next “correct” chord to play and when to, for example, guide the operator to achieving a desired result.

The audio, auditory, or sound messages can include metaphors that allow the operator to predict or anticipate action to be performed by, for example, facilitating operator connection to the vehicle, machine, or equipment control. For example, the operator may anticipate an audio metaphor (e.g., a note or other audio message variations as discussed herein) to adjust operator input to follow a musical signal to, for example, for the musical signal to become or stay in tune. The musical signal can be continuously variable to allow for prediction by the operator, to allow the operator to know what action, including adjustment or direction of adjustment, to perform next, in for example, a sequence of steps to operate the vehicle, machine, or equipment control by anticipating a target communicated via audio message.

The present disclosure can provide for enabling the driver, athlete, or other person with dynamic vehicle or equipment feedback to thereby enhance the ability to control specified vehicle or equipment dynamics within the environment of the vehicle or equipment. The feedback may occur during events, where continuous control input is driving the audible feedback expression. The feedback can also occur after an event, where the assessment of the event is processed and the associated feedback to the driver, athlete, or other person is delivered.

The sound metaphor construction can be dependent on a plurality of vehicle or equipment dynamics and classified events. The overlapping of said events can result in unique expressions. For example, the approach of a braking marker can be influenced by the direction and significance of the wind speed. The resulting expression provides intuitive feedback to the driver on vehicle dynamics with respect to the environments influence, for the purpose of maintaining vehicle or equipment control to achieve performance goals.

The composition and associated construction of the audio metaphor can be dependent on the real time vehicle or equipment dynamics. These include, but not limited to, vehicle or equipment sensor inputs, telemetry, and external data received by the vehicle or equipment. The vehicle or equipment dynamics and associated data can be processed by both digital and analog techniques to identify events, discrete signals, and continuous signals, with results communicated or signaled with audio metaphors. Discrete signals may also be referred to as digital signals. Continuous signals may also be referred to as analog signals. The audible metaphors may include the variation in one or more of frequency, pitch, beat, volume, tone, rhythm pattern, chords, and/or sequence of chords.

The present disclosure can provide for dynamically creating the sound metaphor in response to the continuing variation in vehicle or equipment dynamics and the environment. The driver, athlete, or other person can use the metaphor to adjust driving or operation behavior to achieve a desired tuned metaphor response and/or to maintain the driving or operating behavior to keep within the operating range that produces the continued desired audio metaphor response. A metaphor can involve a significant change in the audio expression, such as where different dynamics contribute to the feedback. For example, a driver may be driving through a corner listening to the metaphor related to achieving optimal wheel slip. However, as the vehicles yaw and/or yaw rate results in either elevated risk or loss of wheel traction, the sound metaphor may introduce a significant variation to the existing metaphor to illicit a driver reaction. The significance of this variation can correspond directly to vehicle dynamics. The correspondence may be directly proportional, exponential, logarithmic or any other function, such that the significance of the variation may be any function of the vehicle dynamics. A comprehensive series of notes and chords can create a metaphor for complex vehicle or equipment dynamics. This can create awareness of movement of the vehicle or equipment as well as allow the driver or operator to adjust operating inputs to result in the sound/music changing to match an optimum or desired outcome (for example, playing a piano to a correct tune/pitch and not miss a beat or a note).

The present disclosure can provide for including the capability to emphasize and/or prioritize the significance of a sound metaphor, in relation to the present vehicle or equipment dynamics and environment. This can include elements to the sound metaphor that are situationally relevant. For example, contamination on the road presents a safety hazard. The audible metaphor(s) may be adjusted, replaced, or cycled depending on the vehicle dynamics and driver's input with respect to the environment. Track marshalling, hazard notification, and/or team instructions can overlap, delay, and/or have priority over other audible and/or haptic messages

The expression of the audio language can be created with benefit to a human's emotional response to audio. The expressions can produce intuitive levels of affirmation, optimism, encouragement, success, calmness, discomfort, pain, fear, anxiety, risk, etc. For example, in motorsport it is desirable to cut the corners where possible to decrease lap times. The curb in the corner however generates an impact force on the car. The vehicle can tolerate a certain impact level (curb strike force). However, hitting the curb with too much force is undesirable and can lead to mechanical failure. Therefore, the sound metaphor associated with curb strike may have a varied expression depending on the nature and magnitude of the curb strike impact. For minimal curb strike, the sound metaphor may emphasis encouragement. For optimal and sustainable curb impacts, the sound metaphor could associate with degrees of success. An unacceptable impact force could result in the metaphor sounding painful. The use of audible metaphor emotion provides an instinctive understanding to the driver and therefore provides an intuitive extension to the metaphor's language.

Feedback to a driver, athlete, or other person with an audible sound metaphor can be related to the result of a completed event. The feedback may also be intentionally delayed to avoid user sensory overload, thereby avoiding distraction when needed or desired and conveying the feedback when the user is more receptive to the information. The delayed time used for delivery of feedback may be predetermined, either by vehicle or equipment location, vehicle or equipment dynamics or time since the last feedback or communication event to the driver. The delayed feedback may also be discarded, where the context for the feedback to the driver or operator may be lost. This condition of discarding a delayed message may be dependent on the accumulated time since the initiating event (e.g., a predetermined time has passed), type of audible metaphor, delivery of subsequent audible metaphors having more context (e.g., based on location), or number or type of other priority communications or messages during the delayed period. In some cases, the user may be a driver. In other cases, the user could be a co-pilot, a simulator user, a team member, and/or a spectator. The present disclosure provides post-event audible metaphors to convey either or both event occurrence and degree of success. Post event metaphor can include, but not limited to, vehicle dynamics related to fuel consumption, energy consumption and/or deployment, tire wear, tire temperature, impact, environment, track position, steering angle, braking, acceleration, race line target, corner apex target, and/or entry/exit speed.

The present disclosure can provide for creating a continuously variable response to driving or operating dynamics. The audio metaphor can incorporate a function to facilitate the driver, athlete, or other person adjusting their vehicle or equipment control to tune to the metaphors desired sound. For example, a discordant combination of notes (a type of chord) represents a non-optimal or undesired vehicle or equipment dynamic condition. As the driver, athlete, or other person adjusts their operating input to the optimal or desired vehicle or equipment dynamic condition, the chord progressively leads to a full and/or desirable harmony. Thereby validating or indicating the proximity to the optimum desired operating range. This type of expression is a harmony-based audio metaphor.

The use of a rhythm and beats can extend the metaphor language by providing situational context. This can prompt the driver's, athlete's, or other person's awareness to the metaphors purpose and provide the starting point for the feedback-based vehicle control. This can pro-actively impact the driver's, athlete's, or other person's behavior to follow the feedback to achieve the driving or operating goals.

The present disclosure can include directional audible effects in relation to the sound metaphors and their associated expression. The audio system may consist of multiple speakers and/or incorporate surround sound technology to enhance the sound metaphor content. For example, the road ahead may have debris, surface contamination, animals, or unexpected water that may present a danger. The associated sound metaphor may incorporate the use of directional or spatial sound to provide the driver with both vehicle and environment awareness of this danger. This concept extends to spatial awareness. This metaphor may be expressed to provide feedback on road position, cornering position, related to vehicle dynamics and the relative position of other vehicles or objects in the environment. A directional audio metaphor may indicate the type of danger, proximity, closure rate, optimal or desired road position, and/or avoidance strategy. The present disclosure can provide for assisting where vision is impaired due to environmental weather conditions, where vision is restricted, where reaction time without advance warning is insufficient, and/or due to the nature of the surface traveled.

The present disclosure can provide for synchronizing with tactile haptics to extend the detail of the feedback to the wearer of the associated haptic device. Haptics can be used to emphasize metaphors, classify different metaphors, or to relate the metaphors to different feedback events. In conjunction with the audio metaphor, the haptics may provide spatial awareness, or relate to different vehicle or equipment systems. For example, a moving vehicle may momentarily hit a retaining wall. The audio metaphor may announce the nature and severity of the impact, while the position of the haptics on the driver may be used as a reference to indicate where the impact was registered on the vehicle.

The present disclosure can provide for enabling a non-driver(s) or non-operator(s) to receive the audio metaphors. This enables the non-driver(s) to experience the vehicle or equipment dynamics represented with audio metaphors in association with the driver's or operator's input. The disclosure provides for a method of education and training for driver to non-driver(s), non-driver(s) to driver, operator to non-operator(s), non-operator(s) to operator, etc.

The present disclosure can provide for the application to recorded vehicle dynamics data to produce audio metaphors for data analysis, such as an extended sensory output to interpret and relate the data to the recorded data. Both the driver and non-drivers (operator and non-operators) can use this for analysis, collaboration and validation of the vehicle or equipment dynamic performance as well as the driver's or operator's control of the vehicle or equipment dynamics. Users can experience the data with audio metaphor feedback at both real time and varied time rates. The operation with recorded data can provide a mechanism to further adjust and improve the behavior and benefit of the audio metaphors, for drivers, non-drivers, and/or operators.

The audio construction can be comprised of either or a combination of music and synthesized content. This can be mixed or used to vary one or more parameters of the audible content to produce the metaphor expression related to different events associated with the vehicle dynamics and environment. A modifier based on a music emotion classification can be used to provide the metaphor in relation to the feedback presented to the driver or operator. This can be comprised of changes in frequency, volume, pitch, beat, vibrato, tone, repetition, and/or scale change. The construction/application and management of the audible metaphors and associated libraries can be built in accordance with the present disclosure.

The Summary is provided to introduce a selection of concepts in a simplified form that are further described below in the Detailed Description. This Summary is not intended to identify key features or essential features of any subject matter described herein.

### BRIEF DESCRIPTION OF THE DRAWINGS

The features of the inventions disclosed herein are described below with reference to the drawings of the preferred embodiments. The illustrated embodiments are intended to illustrate, but not to limit the inventions. The drawings contain the following figures:

FIG. 1 illustrates an example embodiment of a messaging apparatus connected to a vehicle.

FIG. 2 illustrates an example embodiment of a controller and other parts for a messaging apparatus.

FIG. 3 illustrates an example embodiment for generating audio messages.

FIG. 4 illustrates an example embodiment of a process for generating audio messages.

FIG. 5 illustrates an example embodiment of a process for generating audio messages.

FIG. 6 illustrates an example embodiment for prioritizing audio messages.

FIG. 7 illustrates an example embodiment of a process for generating audio messages.

FIG. 8 illustrates an example embodiment of a process for generating audio messages.

FIG. 9 illustrates an example embodiment of a process for generating audio messages.

FIG. 10 illustrates an example embodiment of a process for generating audio messages.

FIG. 11 illustrates an example embodiment of a simulator.

FIG. 12 illustrates an example embodiment of a messaging apparatus.

FIG. 13 illustrates an example embodiment for generating audio messages.

FIG. 14 illustrates an example embodiment for generating audio messages.

FIG. 15 illustrates an example embodiment of a process for generating audio messages.

FIG. 16 illustrates an example embodiment of a process for generating audio messages.

### DETAILED DESCRIPTION

While the present description sets forth specific details of various embodiments, it will be appreciated that the description is illustrative only and should not be construed in any way as limiting. Additionally, although particular embodiments of the present inventions may be disclosed or shown in the context of motorsports, such embodiments can be used as for other sports or vehicles such as downhill skiing, boat racing, and/or trains, respectively. Further, various applications of such embodiments and modifications thereto, which may occur to those who are skilled in the art, are also encompassed by the general concepts described herein.

A gap in time between performing a human action and reviewing a performance report based on that action can impede on the ability of the athlete to identify the particular actions (control inputs) and events that improve or degrade performance. With the amount of data available today, the complexity of transmitting critical information in a readily perceived format is critical. A human's exteroceptive system includes nerves that respond to stimuli emanating from outside of the body via the five basic senses: sight, hearing, taste, smell, and touch. The sense of hearing includes the ability to discern relatively small or large changes in tone, pitch, harmony, frequency, magnitude, urgency, and/or direction. The sense of hearing can be used as sensory input to facilitate and improve performance of various activities described herein while leaving other senses available for the person to perform the activity. Using the sense of hearing can help convey a lot of information while mitigating sensory overload of a human's exteroceptive system.

A system is disclosed for enabling racing vehicle drivers (and in some embodiments, other types of athletes, equipment operators, users, or other persons, machine operators, users, or other persons, and/or drivers) to monitor and improve their performance through audio stimulation. In some embodiments, the system includes an audio device. The audio device can be a speaker, including a speaker of a car stereo system. The audio device can be mounted to a vehicle, a helmet, or any other suitable structure. In some embodiments, the audio device can be coupled to the driver or another piece of equipment, or the audio device could be stand-alone. The audio device could be headphones. The audio device can be electrically coupled to a controller. The controller monitors various parameters of the vehicle's operation (e.g., speed, engine RPM, wheel balance, vehicle location relative to turns, vehicle track position, wheel slip ratio, tire grip, tire temperature), and uses this information to generate audio, auditory, or sound messages that are conveyed to the driver. The system can have a wired and/or wireless transceiver to receive information from one or more data sources, such as a CAN bus of the vehicle, a global positioning system, and/or a computing device that is internal or external to the vehicle.

Depending upon how the system is configured, the system may, via audio signals or messages, convey a variety of different types of information, including real time information, to the driver. For example, the system may signal to the driver when to shift or brake or may notify the driver whether a shifting or braking operation was performed too early or too late. The system can have a user interface for selecting when and what types of audio messages are to be delivered. The system may, for example, be used to provide real time feedback and guidance to drivers during racing or training. The system can replace or supplement other indications the driver may receive during racing or training. An audio message to the driver can minimize distractions to the driver and enhances the time drivers spend with their eyes and attention focused on the road, instead of the control interface.

In some embodiments, the system can prioritize messages for audio delivery to the driver based on message type. The system can prioritize messages based on a user's input before, or during, use. For example, a cue signal based on a trigger event conveying to the driver to perform an action can be prioritized over a feedback signal conveying audio messages regarding driver performance. Further, audio messages received from external sources can be delayed and prioritized to be delivered to the driver after the system determines that there are no other audio messages (e.g., cue signals or feedback signals) to be delivered. Lower priority messages can be postponed and queued to be delivered to the driver once higher priority or more time critical messages have been delivered. Messages can be queued for delivery with a delay, defined track positions or when the recipient is deemed to have a reduced operating cognitive demand.

The system may also use audio messages to, for example, provide cues related the order of tasks to be performed, warn of an impending event, advise of a marshalling condition, provide information related to the position of nearby vehicles, provide information related to environmental conditions, and/or provide instructions regarding crewmember(s) or pit crew positioning such as in a sailing or motorsport context.

Other embodiments of the system and associated methods can be used in other sports to improve performance. For example, in Freestyle Moto X, the system can augment a driver's sense of location to help safely land during a trick being performed. The system can communicate to the driver if enough vehicle speed is being attained to perform a particular trick and/or providing wind direction or speed to instruct/advise driver compensation. Similarly, the system can be used in non-motorized sports such as downhill skiing. The system can, for example, provided real time feedback on the effect of a maneuver on downhill speed.

Embodiments of the system can also be used in non-sport contexts. For example, in one embodiment, the system can provide audio messages conveying to a train engineer if the locomotive is traveling at a safe speed for an approaching corner, bridge or location. The audio device can also act as a reminder to sound the horn when the locomotive is approaching the corner.

As further examples, the system can be used to provide audio cues and audio feedback based on performance of various vehicles and their drivers/operators, such as a racing vehicle, a racecar, motorsport vehicle, commuter or transport vehicle, electric vehicle, hybrid vehicle, construction vehicle, watercraft, aircraft, submersible vehicle, space vehicle, autonomous vehicle, or instructional vehicle (e.g., instructional vehicle operation). The system can also be used to provide audio cues and audio feedback based on performance of simulated vehicles and simulated vehicle's drivers. The system can facilitate operator connection to the vehicle, machine, or equipment control, including in electric vehicles where traditional input cues such as engine revolution per minute and gear selection input are absent or mitigated.

### Current Motorsport Learning Techniques

Motorsports racing currently utilizes trial-and-error learning techniques. A driver will repeat a racecourse many times, analyzing any mistakes made after the fact. Over time, incremental improvements are made until the correct maneuvers become innate. If the driver is talented, the after the fact learning technique may be effective. However, even for a talented driver, this process can often be slow and limited. Only incremental improvement steps are made with each post event analysis iteration. This is a slower and less accurate learning process. Memory is an unreliable source of information and imagining complex data on a screen as real life movement is difficult to comprehend and inconsistent. Further, a driver may simply not be aware of the kinds of inefficient maneuvers being made. The present disclosure discloses a method to provide audio feedback, and audio cues on a near instantaneous or time-delayed manner. The quicker feedback to the driver with associated situational context may enable faster improvement over time. Additionally, instantaneous feedback may allow a driver to improve performance immediately by guiding the driver to learn more efficient actions and choices.

Motorsports is a multi-billion-dollar industry with team spending alone being many billions of dollars. A gain of a fraction of a second in improved performance through new technology can translate into millions of dollars in revenue for a team. Some motorsport regulations do not allow for certain manipulations of car controls using sensors, such as the use of wheel sensors for antilock braking system (ABS) or manipulating ECUs in other ways to gain a competitive edge. Motorsport regulations may allow teams to manipulate the ECUs in limited ways such as providing fuel/energy information or traction control. However, motorsport regulations typically do not restrict the teams from gathering, logging, and analyzing all the information available with permitted or not-restricted car sensors. In some cases, motorsport regulations do not restrict the type of or number of sensors available on a car. In these cases, participating teams can add more sensors to the car if more information about performance is desired.

The regulations allow for the driver to receive available sensor information, including receiving the information in real time. In fact, many types of real time information are already relayed to the driver such as speed, sector times, RPM, flag signals, gear selection, and other messages such as auditory messages from the pit crew. Teams have researched how to increase the types of real time information delivered to the driver. For instance, teams have attempted to deliver to the driver optimum and/or desired shift points by visual cues, such as green, yellow, red flashes. This research has shown that in some cases, they visually compete with other signals for the driver's mental bandwidth, and the driver may filter out the signals during periods requiring focused hand eye coordination. Yet, the extra information to optimize performance can be vitally important to improve lap times, reduce testing costs, increase safety, reduce tire wear, and enhance pit stop procedures. Teams that can effectively deliver the needed performance information to all team members, including the driver, can increase performance leading to increased team revenues while reducing team costs. Delivering real time information through audio cues and audio messages can reduce or eliminate the need for a user of a messaging apparatus to view or listen to the performance information in a post event analysis. In some cases, the audio signals can include sophisticated audio metaphors for operator feedback control.

By sending real time data, a driver can learn and self-correct more accurately and quickly. The driver can immediately respond to an audio signal and quickly learn the effects of changing particular body movements or control inputs on the response of the motorsport or racing vehicle, such as a racecar. The human performance factor of motorsports is still preserved while providing a hypersensitive “feel” of the vehicle and associated subtle dynamics. As a driver learns and improves over time, the driver may need audio cues on a less frequent basis. As a driver's skill develops, the system can be configured to reduce the audio cues used and/or replace the audio cues with different audio cues to further develop the driver's performance. The system can be configured to focus the audio cues on improving a particular skill or operation.

### Messaging Apparatus for Delivering Audio and Other Messages Based on, for Example, Data Generated by Vehicle Sensors and Externally Generated Data.

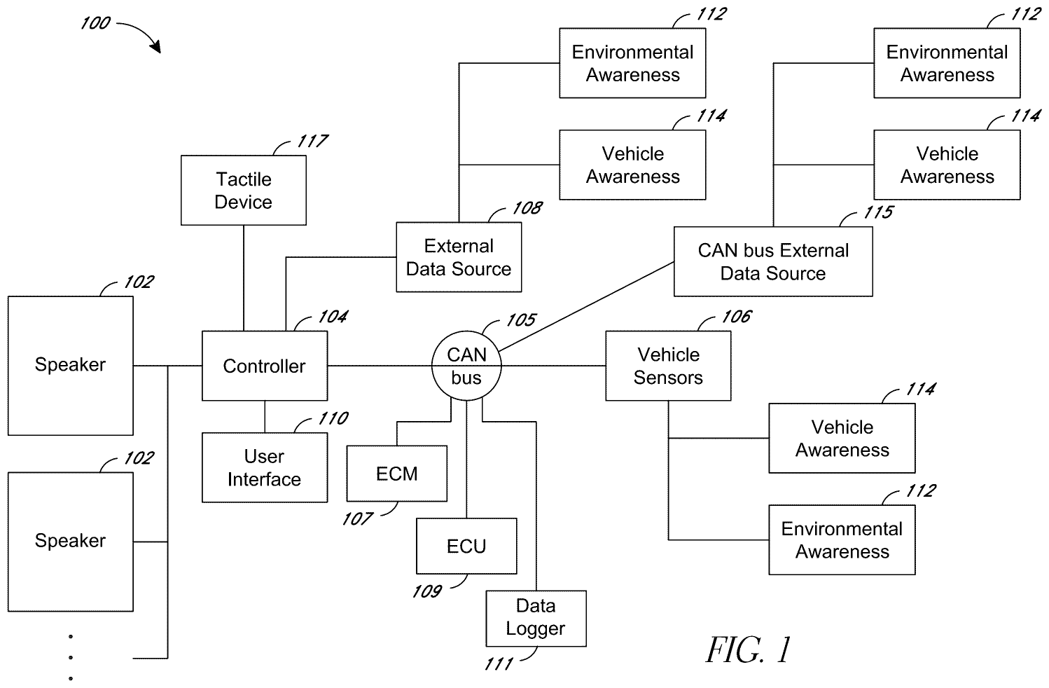

FIG. 1 illustrates an example embodiment of a messaging apparatus 100 for enabling racing vehicle drivers to monitor and improve their performance through audio feedback and cue messages. In some embodiments, the messaging apparatus 100 can be an audio apparatus that communicates audio or auditory messages. In some embodiments, the messaging apparatus 100 can be an audio apparatus in communication with a haptic apparatus that communicates haptic or vibrational messages as discussed herein. The messaging apparatus 100 includes a controller 104. The controller 104 is coupled to one or more speakers 102, and a user interface 110. The controller 104 can be connected to the car's CAN bus 105 and monitors various parameters of the vehicle's operation (e.g., speed, RPMs, wheel balance, location relative to turns). The CAN bus 105 is the car's central information system that links the car's electronic components such as an ECM (Engine Control Module) 107, ECUs (Electronic Control Units) 109, sensors 106, and a data logger 111. The ECM 107 can control engine functions such as fuel injection and rev control limits. The car sensors 106 can detect car movements such as wheel spin, while the ECUs 109 can control, within certain limits, the wheel spin based on signals from the sensors 106 to provide, for example, traction control. While a car may already have many sensors 106, where not prohibited, teams can add additional car sensors 106 depending on data that is desired to be collected. For example, a team may add a sensor 106 to measure brake line pressure if a car does not already have a sensor 106 monitoring this variable. The data logger 111 can log data from the ECM 107, ECUs 109, and/or sensors 106 that is transmitted through the CAN bus 105. The vehicle sensors 106 can receive vehicle awareness data 114 and/or environmental awareness data 112. The controller 104 may be functionally or operatively connected to an external data source 108. In some embodiments, the external data source 108 can be functionally or operatively connected to the controller 104 via the CAN bus 105. In some embodiments a CAN bus external data source 115 is connected to the CAN bus 105. The CAN bus external data source 115 can include vehicle awareness data 114 and/or environmental awareness data 112. The connection may be a network connection. The network connection may be an Internet connection, including via Wi-Fi, or other suitable communication protocols such as Bluetooth, cellular protocols, etc., as well as wired connections between various components described herein. The external data source 108 may include vehicle awareness data 114 and/or environmental awareness data 112. In some embodiments, the controller 104 may be connected to a tactile device 117.

In some embodiments, the stereo system of the racing vehicle includes the speaker 102. In some embodiments, a helmet configured to be worn by the driver of the racing vehicle includes the speaker 102. Voice communications such as from other team members may be included and prioritized as discussed herein with the audio event metaphors.

In some cases, vehicle awareness data 114 may be racing vehicle awareness data. Racing vehicle awareness data, or racing vehicle awareness, may be information regarding the location of the racing vehicle relative to the road or racetrack and/or the position of the racing vehicle relative to a corner (i.e. cornering position) of the road or racetrack. Audio messages corresponding to racing vehicle awareness may include one of a desired road racetrack position of the racing vehicle or a location of impact on the racing vehicle. Location of impact on a road or racetrack may relate to the location of an impact on the racing vehicle when the racing vehicle is cutting a corner. A racing vehicle may cut a corner to reduce lap time, including via achiever a corner apex target as discussed herein.

Environmental awareness data 112, or environmental awareness or environmental information of a racing vehicle may include at least one of position of another racing vehicle relative to the racing vehicle or the position of an object in an environment of the racing vehicle. This information could be gathered via sensors mounted on the racing vehicle, such as vehicle sensors 106 or the information could be transmitted to the racing vehicle from an external data source 108, located external to the vehicle. Audio messages corresponding to the environmental awareness may include a cue message to avoid the other racing vehicle or the object in the environment of the racing vehicle. The audio messages corresponding to the environmental awareness may include a cue message corresponding to, relative to the racing vehicle, at least one of a proximity or approach rate of the other racing vehicle or the object in the environment of the racing vehicle. The message may also indicate a type of object in the environment of the racing vehicle.

In some embodiments, the messaging apparatus 100 may be a system for improving racing vehicle driver performance. The speaker 102 communicates audio signals and/or messages to a driver of a racing vehicle or other persons such as passengers in the vehicle or remote persons such as pit crew members or persons in a simulator or other computer systems (to which audio signals and/or messages can be communicated to in real time or post event for evaluation).

The controller 104 controls the speaker 102 to communicate performance-related information to the driver during movement of the racing vehicle. The controller 104 may be configured to receive vehicle information. The vehicle information may include vehicle information collected by vehicle sensors 106 of the racing vehicle. The vehicle information, and the other data described above can be used to select and/or generate audio messages to convey to the driver. The messages may be cue messages or post-event feedback messages. The cue messages are intended to assist the driver in timing actions correctly and performing maneuvers properly or as desired. The post-event feedback messages are intended convey to the driver whether actions were performed properly or as desired. The vehicle information and the other data can be used to determine which audio messages to send, and which audio messages to prioritize.

In some embodiments, the audio device or messaging apparatus 100 may be configured to have at least a portion physically contact the driver (e.g., be positioned on the driver or a body part of the driver), the audio device may include the speaker 102 capable of communicating audio signals and/or messages to the driver.

In some embodiments, the messaging apparatus 100 is a system for improving control of a vehicle. It can include a speaker 102 capable of communicating audio signals and/or messages to a person. It can also include a controller 104 that controls the speaker 102 to communicate performance-related information to the person. The controller 104 can receive vehicle information and use the vehicle information to communicate to the person an audio message that notifies the person of a desired timing for performing a driver action corresponding to controlling the vehicle. The vehicle information can be supplied from an external data source 108, through the CAN bus 105, or directly from sensors 106 connected to the controller 104.

The messaging apparatus 100 can be used in conjunction with various vehicles such as a racing vehicle, racecar, motorsport vehicle, commuter or transport vehicle, electric vehicle, hybrid vehicle, construction vehicle, watercraft, aircraft, submersible vehicle, space vehicle, autonomous vehicle, or instructional vehicle.

### The Controller for Messaging Systems

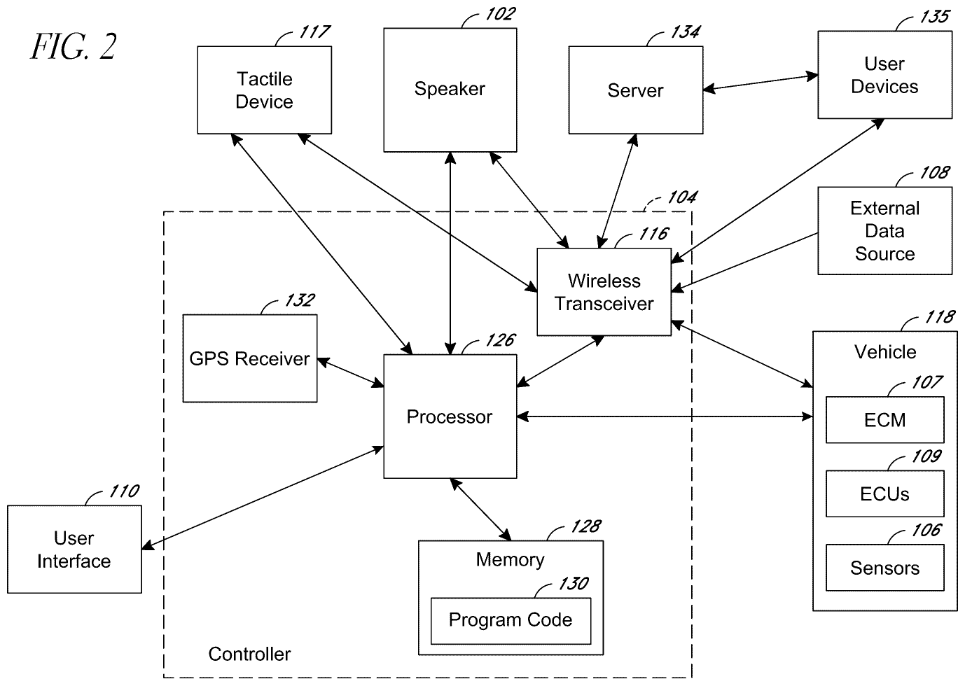

FIG. 2 illustrates a block diagram showing elements of an example embodiment of a messaging apparatus 100 with a controller 104. The controller 104 can be in the form of any suitable computing device or board and is typically a portable device that attaches to the speakers 102. The controller 104 functions and features as discussed herein can be integrated into the existing vehicle ECM 107, ECU 109, and/or other vehicle hardware. In the illustrated embodiment, the controller 104 has a processor or hardware processor 126 and a computer readable memory 128 that stores program code 130. The program code 130 can include specific computer-executable instructions, which when executed by the process 126, cause the messaging apparatus 100 to perform desired functions and operations as discussed herein, including communicating audio and/or haptic communication messages or metaphors. The program code 130 implements the algorithms and program logic for performing the various functions and operations described herein, including the analysis of vehicle-related information and the generation of audio signals to be applied via the one or more speakers 102.

The controller 104 may be powered by a dedicated battery or by the vehicle's battery. The controller 104 can be integrated with the one or more speakers 102, or can be outside of, and functionally linked to the one or more speakers 102. The processor 126 can send information to and receive information from, the CAN bus 105 and the external data sources 108. The processor 126 can be connected to a vehicle 118 to receive signals from the CAN bus 105. The CAN bus 105 connected to the ECM 107, ECUs 109, and/or sensors 106 within the vehicle 118. In some embodiments, the processor 126 can receive signals from the vehicle 118, but cannot send signals to the vehicle 118 or otherwise directly control the vehicle. The processor 126 is preferably a microprocessor or microcontroller, but may alternatively be, for example, an application-specific integrated circuit (ASIC), FPGA, or other device that includes application-specific circuitry. Although a single processor 126 is shown, the controller 104 may include multiple distinct processors in some embodiments.

In some embodiments, the processor 126 can be connected to a GPS (global positioning system) receiver 132 to receive GPS information as described herein. The processor 126 can send information to and receive information from the GPS receiver 132. The processor 126 can be connected to a user interface 110 to receive user input as described herein. The processor 126 can send information to and receive information from the user interface 110. In some embodiments, the user interface can be connected to the processor 127 via the wireless transceiver 116. The user input device 110 can include, for example, a keypad and a display, or a touchscreen, for enabling the driver or driver team to view and adjust various configuration settings. In some embodiments, the user interface can also be controlled through existing vehicle controls or remotely through an external interface. For example, in some embodiments, steering wheel controls can be programmed to their respective switch position via the CAN bus 105 to the controller 104, and the signals could be used as user inputs to the controller 104. The controller 104 could additionally or alternatively support voice recognition, gesture recognition, and/or other types of user input. In embodiments where the user interface 110 includes a software-based user interface, the user interface may display status information of the messaging apparatus 100 based on the information sent to the user interface by the processer 126.

In some embodiments the messaging apparatus 100, and specifically the controller 104, may use lap distance to determine a track position of the vehicle. Lap distance can be the elapsed distance from a lap reset point to the present location of the vehicle. The lap distance can be used to determine an approximate track position. Track position may be estimated in a variety of ways. Estimating track position may be based on steering data, inertial sensor data, and/or other data available to the controller 104.

As illustrated, the processor 126 may be connected to a wireless transceiver 116. The wireless transceiver 116, if present, enables the processor 126 to communicate wirelessly with the vehicle 118, to one or more speakers 102 (and/or tactile actuators on a tactile device 117). The one or more speakers 102, the external data source 108, the internet, and/or a pit crew computing device that is external to the vehicle. The wireless transceiver 116 can also enable the processor 126 to wirelessly communicate with the GPS receiver 132 and/or the user interface 110. Although a single wireless transceiver 116 is shown, separate, wireless transceivers may be provided for communication with different systems or entities. The wireless communications, if any, with the vehicle 118, speakers 102, and/or different systems or entities may either replace or supplement wired communications.

The controller 104 can receive sensor signals from the CAN bus 105 through a transceiver using any appropriate signal interface(s) and protocol(s). In some embodiments, the transceiver may be a wired transceiver. In some embodiments, the transceiver may be a wireless transceiver, such as wireless transceiver 116. Preferably, the controller acts as a passive node on the CAN bus 105, meaning that it monitors signals on the CAN bus 105 but does take any action that directly affects the vehicle's operation. The controller 104 can be used to broadcast message data and message delivery timing on the CAN bus 105. The information can be subsequently recorded with the vehicle data logger for later analysis. The recording of this data can be used to verify that audio messages are being delivered as intended, and the data can be useful for improving the system. The controller 104 can determine the audio signals to be delivered to the driver based on the data received from the CAN bus 105 and based on various programmatic criteria. In some cases, the controller 104 may also use other sources of information (other than the CAN bus) to select audio signals to apply. For example, the controller 104 may include a GPS device that uses both GPS signals and local beacons to accurately determine the car's real time location; in such embodiments, the controller 104 may use this additional source of information to, for example, select shifting-related audio signals to transmit to the driver.

In embodiments in which a wireless transceiver 116 is provided, the controller 104 can also receive an audio signal via the wireless transceiver 116 to be communicated to the driver through speakers 102. For example, a team member may send a known or predetermined audio signal to the driver instead of communicating through a standard radio. The wireless transceiver 116 can be part of the controller 104 or any other part of the system depicted in FIG. 1 such as being directly connected to the CAN bus 105. In some cases, the wireless transceiver 116 may be capable of communicating with a device that is external to the vehicle; where this is the case, another team member or another controller outside the system depicted in FIG. 1 can determine and send an audio signal to the driver through the wireless transceiver 116. The other team member may also be able to send data to the controller 104 to indicate that the audio messages being generated by the system should be adjusted.

The speaker 102 can be any system capable of delivering an audio message, metaphor, or signal to a person.

In some embodiments, the controller 104 can be configured to receive input information, which can include vehicle information, execution information, auxiliary information, and/or user information. The classification of the input information does not limit the functionality of the embodiments described herein. Rather the classification of the input information is for discussion purposes. The vehicle information, also referred to as signal sensor data or vehicle sensor information, can be based on signals from vehicle sensors. The execution information can be based on signals from vehicle sensors and/or signals from auxiliary sensors such as a global positioning system. The auxiliary information can be based on signals from auxiliary sensors such as pit crew information. Vehicle information can, for example, overlap with auxiliary information because, for instance, individual wheel speeds as determined by tire rotation may not match the speed of the vehicle as determined by global positioning system information and/or inertia movement sensors. This discrepancy may stem from wheel slip relative to the surface of travel. Execution information can be the same as auxiliary information such as for acceleration. Acceleration can be execution information as determined by the acceleration a driver achieves with reference to a maximum acceleration that could have been achieved based on optimum and/or desired balance of a vehicle weight on all four tires. Acceleration can also be auxiliary information based on actual vehicle acceleration relative to the surface of travel as determined by a global positioning system and/or inertia movement sensors. Vehicle information may be communicated to the controller 104 by the vehicle sensors 106, by an external data source 108, or by the CAN bus 105. Data may also be sent to the controller 104 by the user interface 110. The external data source 108 may include data from teammates or data from a server.

The vehicle information can include characteristics of vehicle operation such as power use, fuel/energy consumption, wheel speed as determined by tire rotation, wheel spin relative to other wheels, wheel slip, revolutions per minute of the engine, gear selection, accelerator pedal position, brake pedal position, brake pad position, brake line pressure, steering angle, transitional input ramps, vehicle weight distribution, and vehicle momentum, which can be used by the controller to generate audio signals as described herein. The execution information, sometimes referred to as performance data, can include time gain or loss from reference laps, time gain or loss from reference points, timing of gear selection, braking rate, acceleration, steering angle, entry and exit speeds, entry and exit lines, entry and exit decelerations and accelerations, and grip use, which can be used by the controller to generate an audio signal as described herein.

The auxiliary information can include input from a global positioning system, vehicle location, vehicle speed, tire wear, acceleration, deceleration, radius for approaching corner, speed for approaching corner, pit crew information, and reference points including for braking, steering angle, acceleration, and approaching corners, which can be used by the controller to generate an audio message as described herein. Based on the received input information, the controller 104 can generate an audio message in real time or near real time, at a predetermined time, at certain racetrack locations, and/or at particular time intervals, which may be identical or different. In one embodiment, user(s) can query an input device for real time or near real time information. All the vehicle related data may sometimes be called performance data or vehicle performance data. Data related to operator inputs may also be called performance data or driver performance data.

The user information can include information from a driver and/or at least one other user able to communicate with the messaging apparatus 100. User information may also be referred to as user preferences. In an embodiment, either a driver or another user can configure the messaging apparatus 100 with, for example, an input device to generate or communicate a certain audio signal, or choose from a list of audio signals or profiles to determine the type or contents of audio signals to be communicated. Then another user can choose from a list of available audio signals, using, for example, a pit crew computing device, to communicate a message to a driver. This can be helpful to avoid, for instance, conventional auditory or visual communication that can lead to slower lap times. In embodiments in which the controller 104 includes a GPS receiver 132, the controller 104 can take into consideration vehicle's current and past location in determining whether and when to generate particular audio signals. In some cases, the GPS receiver 132 (or a separate wireless transceiver 116 provided on the controller 104) may be configured to receive location-based signals transmitted by fixed beacons positioned along the racetrack. These signals may enable the controller 104 to more accurately determine the vehicle's position than standard GPS.

In some embodiments, choosing from a list of profiles can allow a driver to customize the types of audio signals generated. The profiles can include options to receive selected specific audio signals, for example, such as receiving audio signals regarding optimum and/or desired gearshift points and optimum and/or desired corner entry speeds, but not receiving audio signals regarding reference points for braking, acceleration, and steering angle. Other profiles can include options to receive audio signals that are more relevant depending on the driving conditions such wet or dry conditions and/or practice run versus an actual race. Further profiles may be related to managing fuel/energy consumption, managing tire temperatures and tire wear, or managing strain on other vehicle components. The profiles may also include an option to disable some or all of the audio signals. Reducing or disabling the audio signals may be advantageous for evaluating change in driver learning or performance when exposed to audio signals.

In some embodiment, the driver can change profiles in real time or near real time, at a predetermined time, or at particular time intervals, which may be identical or different. Profiles can help improve performance by, for instance, providing additional information about the car to the driver during wet conditions when actual feedback of the car's movements is subdued or reduced due to slippery conditions. A wet condition profile can help a driver perform or repeat body controls more accurately and consistently as if driving during dry conditions. In some embodiments, the controller can also be placed into a “learn” mode in which it observes the driver's performance over several laps and then determines (or recommends) the types of cues and other signals to be generated. In some embodiments, the profiles can also be externally controlled the race team. This may facilitate audio cue development, refinement, and/or audio cue efficacy evaluation.

In some embodiments, the profiles may generate different sounding audio messages for any given data condition. For example, a first profile may have a first base track that is modified depending on the performance data of the vehicle, a second profile may have a second base track that is modified depending on the performance data of the vehicle. The first and second track are different. For the same performance data, profile A will produce a different audio track than profile B. Similarly, other conditions related to how the audio tracks are generated can be varied and associated with profiles. For example, in profile A, audio characteristic A may vary with respect to changes in performance data A, whereas in profile B, audio characteristic B may vary with respect to changes in performance data A. In different profiles the relationship governing the degree of change of an audio message may be different. For example, in profile A, audio characteristic A may vary linearly with respect to changes in performance data A, whereas in profile B, audio characteristic A may vary exponentially with respect to changes in performance data A. In some embodiments, performance data A may be tire wear data. In some embodiments, audio characteristic A may be volume. In some embodiments, audio characteristic B may be beat.

Various profiles, as mentioned above, may also be used by various user devices 135. Various user devices may be in communication with the server 134 or the wireless transceiver 116. The user devices 135 may receive audio messages from the controller 104 or the server 134. The user devices 135 may also receive data suitable for generating audio messages and/or other data. In some embodiments, the user devices 135 may be configured to convert received data into audio messages. The audio messages generated by the user devices 135 may be different from the audio messages generated by the controller 104. The audio messages generated by a plurality of user devices may all be different. The user devices 135 may comprise systems similar to controller 104.

In some embodiments, the user devices may also accept user input to control the system and/or provide inputs into the system as discussed herein. For example, the system can be enabled, disabled, adjusted, or otherwise controlled such as adjusting audio metaphors as discussed herein via a user device 135 by a user such as a non-driver, a non-operator, or a non-athlete, including engineers or other team members. Such users can also enable, disable, adjust, or otherwise control the system via the user interface 110.

In some embodiments, the controller 104 can adaptively fine tune the audio signals based on an automated assessment of the driver's strengths and/or weaknesses. For example, if the controller detects that the driver is consistently late at performing a particular action (e.g., braking or shifting), the controller 104 may start generating associated audio signals for performing this action, or may adjust the timing and/or intensity of such cues. Adaptive learning can emphasize certain aspects of training or mitigate a driver's weaknesses or failure to achieve the action. The profiles discussed herein can also include profiles ranging from novice to expert drivers, such as novice, intermediate, advanced, or expert. In one embodiment, a novice profile can include most if not all audio signals described herein to be communicated to a driver. An expert profile can include counterintuitive audio signals or audio signals that help to counteract the particular weaknesses of a driver. An example of a counterintuitive audio signal can be approaching a corner on the racetrack at a different entry position, angle and speed, in order to exit the corner at a higher speed to gain increased higher top speed benefit in the following track section. The adaptive control may use machine learning. The adaptive control may include various determinations executed on a server, such as server 134.

The controller 104 may also receive and analyze other sources of information, such, for example, global positioning data, beacon data, predetermined messages, etc. The connection to the controller 104 can be wireless through a wireless transceiver 116. Depending upon how the system is configured, the system may, via audio messages or signals, convey a variety of different types of information, including real time information, to the driver. For example, the system may signal to the driver when to shift or brake or may notify the driver whether a shifting or braking operation was performed too early or too late. The system may, for example, be used to provide real time feedback and guidance to drivers during racing or training. The system can replace or supplement other indications the driver may receive during racing or training. The messaging apparatus, or parts thereof could be mounted to a vehicle, a helmet, or any other suitable structure. In some embodiments, the audio device could be coupled to the driver or another piece of equipment. The messaging apparatus, or parts thereof, could be stand-alone. The messaging apparatus 100, or parts thereof could be included in headphones. The audio device could be in contact with the driver. The speakers 102 of the messaging apparatus 100 could be located far from each other, could be proximal to or touching various people, and could all include their own controllers 104. The speaker function may be also implemented using bone conduction transducers.

In some embodiments, the one or more speakers 102 include two or more speakers and the audio messages 304 may include directional, spatial, 3D surround, or other similar sound corresponding to at least one of vehicle awareness data 114 or environmental awareness data 112 of the racing vehicle. The directional sound may correspond to data received from the external data source 108. Speakers 102 more than two speakers may be included. The speakers 102 could be distributed around the vehicle to enable audio messages to include finer directional, spatial, 3D surround, or other sound. The system may use audio processing techniques to mimic the effects of 3D audio. Headphones and helmets may comprise a multitude of speakers to provide directional, spatial, 3D surround or other sound.

In some embodiments, the controller 104 may associate tactile messages with the audio messages, the tactile messages corresponding to at least one of emphasis of the audio messages, classification of the audio messages or relating the audio messages to different post-event feedback messages. The controller 104 may be configured to associate tactile messages with the audio messages. The tactile messages may be delivered along with directional sound corresponding to at least one of racing vehicle awareness or environmental awareness of the racing vehicle. The tactile messages may be delivered to the receiver via the tactile device 117.

### Audio and Other Message Generation

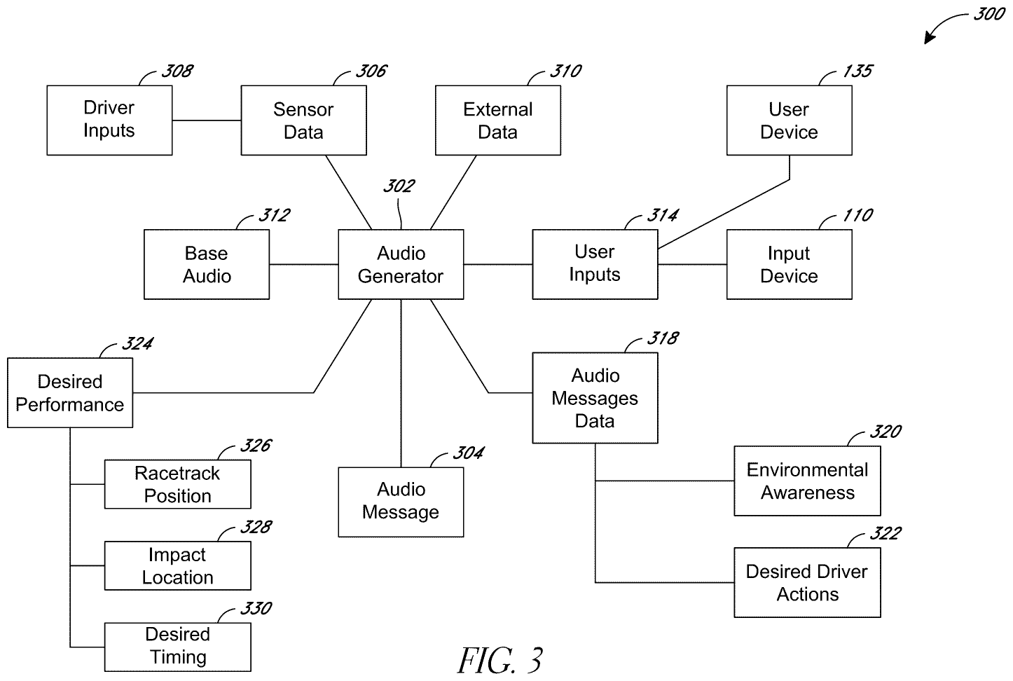

FIG. 3 illustrates an example embodiment for generating audio messages 300 with audio metaphor generation program modules, of which corresponding program code can be stored in memory 128 and executed by the processor 126. The controller 104 can cause the system to communicate one or more messages as discussed in reference to FIG. 3 herein. The one or more components described in reference to FIG. 3 can be modules of the controller 104.

The outputs from the audio metaphor generator program modules can be used to send an audio signal to the one or more speakers 102. The audio modifier or generator module 302 generates the audio message 304. While identified as the audio modifier or generator 302, the generator 302 can be a haptic or other message generator 302. The generator 302 can generate haptic or other messages as discussed herein for audio messages, but having haptic or vibration specific implementations, with audio messages having unique characteristics and implementations as discussed herein different from haptic messages. The generator 302 can be considered a message or metaphor generator 302 that can generate audio and/or haptic messages or metaphors as discussed herein.

The audio message 304 can be delivered to the recipient using the one or more speakers 102 or any another suitable sound generating device 102, including electroacoustic transducers. In some embodiments, the audio message 304 may be saved for later playback. Audio message 304 may be a discrete message, or a continuous audio stream. The audio message 304 may indicate how well an action was performed, when an action was performed, when an action was performed compared to a benchmark, when an action should be performed, how the car is performing, the status of the various vehicle systems, the rate of change of the various vehicle systems, how the driver is performing, how the driver is performing versus a certain or predetermined benchmark performance, how the environment of the vehicles surroundings are changing, how the driver should respond to the vehicle's surroundings, or various other messages as described herein.

The audio modifier or generator 302 can use a variety of source data to generate audio messages 304. The audio modifier 302 may receive a base audio 312, sensor data 306—the sensor data 306 may include data from the CAN bus 105 as well as driver or operator input data 308, external data 310, user inputs or preferences data 314—some of which may be input via the user interface 110 or the user device 135 as discussed herein by an operator and/or non-operator, including driver and/or non-driver, cue messages data—such as environmental awareness data 320, or desired driver action data 322, and desired performance data which may include racetrack position data 326, impact location data 328, and/or desired timing data 330. The base audio 312 may be an audio file. The base audio 312 may be a set of parameters suitable for conversion to an audio file. The base audio 312 may be data suitable for generating an audio message.

Various audio messages can be generated using the audio modifier 302. In some embodiments, the audio messages may include variations in one or more of frequency, pitch, beat, volume, tone, rhythm pattern, chords, and/or sequence of chords. In some embodiments, these variations may be applied to a base audio 312. In some embodiments these variations are used to generate an audio file without using a base file. The vehicle information about the vehicle may include external data 310 received by the vehicle. The vehicle information may also include information about the environment of the vehicle, such as environmental awareness data 320. In some embodiments, the audio messages are adjusted, played, or cycled based on the external data received and the priorities defined.

In some embodiments, the controller 104 is configured to make the audio messages harmonious in response to the racing vehicle being operated within a desired operating range. In some embodiments, this operating range may relate to a single vehicle dynamics parameter lying within the desired operating range. The vehicle dynamics parameter may be communicated to the audio modifier 302 as a component of external data 310 or a component of the cue or feedback messages data 318. The desired value, or desired operating range may be communicated to the audio modifier 302 as desired performance data 324. In some embodiments, the audio messages may be made harmonious in response one or more various vehicle dynamics parameters and/or environmental parameters being within an optimal range. In some embodiments, the controller 104 is configured to generate a cue or feedback message that varies from the harmonious audio messages in response to the racing vehicle being operated outside the desired operating range. The system may be configurable to adjust the desired operating range either on the fly or in between uses. The system may be configurable to adjust the desired operating range in real-time or in near real-time. In some embodiments, this adjustment can be entered via the user interface 110. In some embodiments, the adjustment may be submitted to the system via the wireless transceiver 116. In some embodiments, the adjustment may be entered via adaptive control of the system. Adaptive control may include automatic self-adjustment by the system. Self-adjustment by the system may be controlled by machine learning. The extent of the variation of a cue or feedback message can correspond to an extent of the racing vehicle being operated outside the desired operating range. The variation may correspond to a variation from a base audio 312. The extent of the variation of the cue or feedback message can correspond to the extent of the racing vehicle being operated outside the desired operating range. The controller 104 can be configured to make the audio messages out of tune in response to the racing vehicle being operated outside a desired operating range. The configuration of the controller 104 as discussed herein may, in some embodiments be achieved using the processor 126 or the server 134.

In some embodiments, the audio signal or audio message 304 corresponds to at least one of the environment, track position, entry speed, or exit speed of the racing vehicle. The audio signal can be adjusted depending on user inputs. Cue messages regarding actions to be performed by the driver can be continuously varied in response to actions performed by the driver or the environment of the racing vehicle. The audio signal can have an audio pattern representing a late driver-initiated event. This audio pattern can be applied in response to a determination that the driver-initiated event occurred more than a threshold amount of time after the desired timing. An audio signal can have an audio pattern representing an early driver-initiated event. This audio signal can be applied in response to a determination that the driver-initiated event occurred more than a threshold amount of time before the desired timing. Desired timing can be determined by the computing device based at least partly on vehicle location data. The desired timing, and the actual timing achieved may be supplied to the audio modifier 302 as desired performance data 324, or as cue or feedback message data 318.

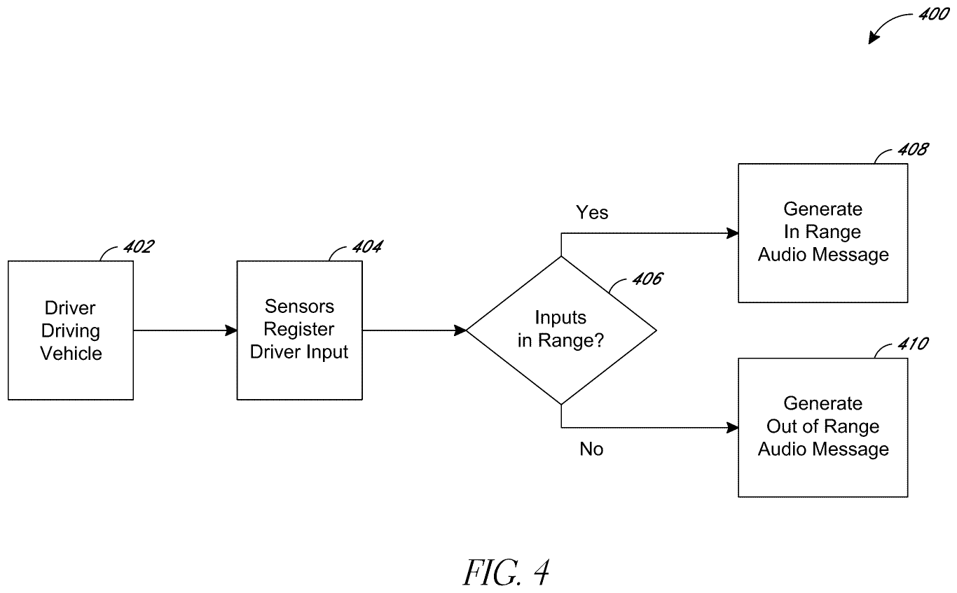

FIG. 4 illustrates an example process 400 for generating audio messages. FIG. 4 can be an example embodiment of a process for providing audio feedback to an operator based on whether inputs are within a given range. The process 400 may be part of the audio modifier 302 that can be executed on the processor 126. At block, 402 the driver is driving the vehicle. The vehicle generates data and at block 404 the vehicle sensors 106 register the driver inputs and the other vehicle data. At block 406, the system determines if the inputs are in a preferred range. If the inputs are with the preferred range, the system may generate an in-range audio message at block 408. If the inputs are not within the preferred range the system may generate an out-of-range audio message at block 410. The actions performed by the driver may include at least one of steering, gear shifting, braking, regenerative braking, energy harvesting, deployment of energy, or acceleration. The driver may perform these actions at block 402.

In some embodiments, energy management may include anticipating strategical harvesting and deployment of energy. For example, desired steering angle may be controlled to optimize energy usage by, for example, reducing or optimizing desired tire wear. If the driver oversteers, then the driver may have to correct for the oversteer while the performance threshold of the tire may have been crossed or surpassed. With oversteer, the tire performance may have dropped or been hampered (e.g., excessive tire wear), leading to suboptimal energy usage or deployment of the tire for that particular maneuver (e.g., taking a corner). Accordingly, audio messages as discussed herein can communicate desired tire performance management to not over utilize tire capacity (e.g., excessive tire wear) that can, for example, lead to a flat tire or an underperforming tire that needs to be changed out due to excessive tire wear beyond a desired or predetermined threshold.

Similar control scenarios using audio messages as discussed herein can be applied to communicate, for example, desired fuel/energy consumption (e.g., throttle control to mitigate excessive fuel/energy usage for a particular maneuver such as during acceleration), desired brake life and/or usage (e.g., brake position control to mitigate excessive use of brake capacity/life for a particular maneuver such as during braking), desired regenerative braking (e.g., optimize energy harvesting to mitigate excessive brake capacity/life usage for a particular maneuver such as during braking), and/or desired gear shifting (e.g., optimize gear shifting to mitigate over steering, fuel/energy consumption, brake life usage, or suboptimal regenerative braking). The system can use audio messages to communicate to the driver/operator how to optimize or achieve desired deployment of energy, which can include any one of or a combination of, for example, desired steering angle, desired tire wear, desired fuel/energy consumption, desired brake life and/or usage, and/or desired gear shifting as well as other machine operation/controls discussed herein.

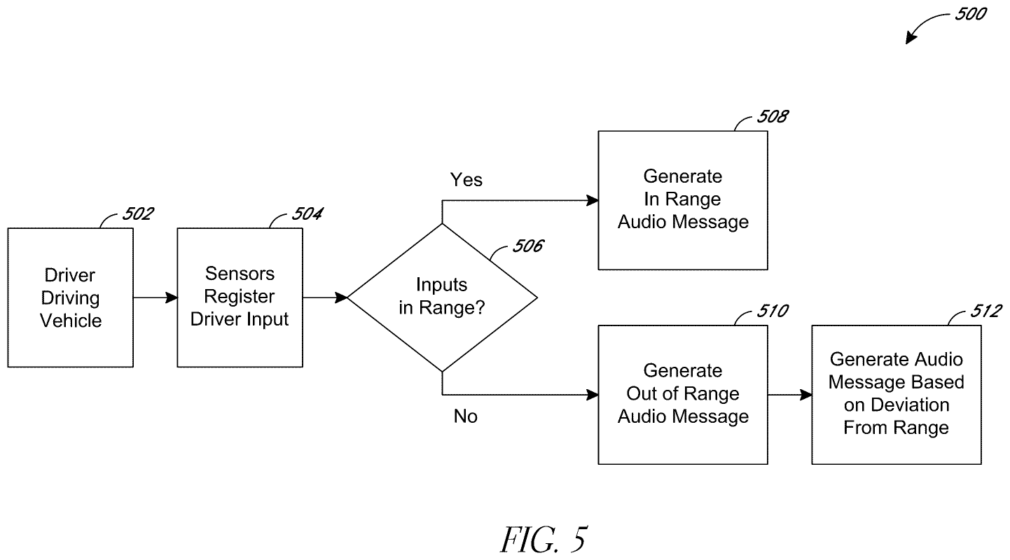

FIG. 5 illustrates an example process 500 for generating audio messages. FIG. 5 can be an example embodiment of a process for providing audio feedback to an operator based on whether inputs are within a given range and how far out of the range the inputs are. The process 500 may be part of the audio modifier 302 that can be executed on the processor 126. At block 502, the driver is driving the vehicle. The vehicle sensors generate data based on vehicle operations and at block 504, the vehicle sensors 106 register the driver inputs and the other vehicle data. At block 506, the system determines if the inputs are in a preferred range. If the inputs are with the preferred range, the system may generate an in-range audio message at block 508. If the inputs are not within the preferred range the system may determine the extent to which the inputs deviate from the preferred range in block 510. At block 512, the system may generate an audio message based on the extent of the variation from the preferred range, of the driver's action. The actions performed by the driver may include at least one of steering, gear shifting, braking, regenerative braking, energy harvesting, deployment of energy, or acceleration. The driver may perform these actions at block 502.

The controller 104 may be configured to communicate the audio messages corresponding to performance thresholds of the racing vehicle. The performance threshold may relate to at least one of wheel slip ratio, tire grip, tire wear, tire temperature, curb impact force, acceleration rate, deceleration rate, cornering rate, dynamic vehicle movements, and/or fuel/energy consumption. Fuel consumption may be related to the use of petrol/gas for an internal combustion engine vehicle. Energy consumption may be related to the use of electrical energy (e.g., from a battery) for an electric vehicle. Audio messages may also correspond to at least one of environment of the racing vehicle, track position, or entry or exit speed. The audio messages can be adjusted depending on user inputs. Cue messages regarding actions to be performed by the driver may be continuously varied in response to actions performed by the driver. Cue messages regarding actions to be performed by the driver may be continuously varied in response to changes in the environment of the racing vehicle. The cue messages may vary in at least one of rhythm or beat to prompt an action to be performed by the driver. The variation in message composition may be generated by the audio modifier 302

In some embodiments, the messaging apparatus 100 may include more than one speaker 102 capable of communicating audio signals to a person. The controller may be configured to select audio messages to convey to the person. The audio modifier 302 may generate and/or select audio messages to be delivered to the user. The generated and/or selected audio messages may include cue messages regarding actions to be performed by the driver. The selected audio messages may include post-event feedback messages that convey to the driver whether actions were performed properly or as desired. The audio and/or haptic message may be communicated to a person substantially in real time as the driver controls the racing vehicle. Audio and/or haptic messages that are communicated in real time may be considered a post-event feedback message (or feedback message) corresponding to the driver's inputs or driver-initiated event communicated in real time as the driver operates the vehicle via driver inputs. Feedback messages communicated in real-time can be considered post-event feedback messages because even though communicated in real-time, the audio and/or haptic message is in response to driver input/action or driver-initiated event that may have occurred a fraction of a second earlier and may still be occurring as the feedback message is communicated to the person. A post-event feedback message can include that the driver input/action or driver-initiated event that caused the feedback message to be generated by the controller 104 as discussed herein is still occurring (e.g., the driver is still performing the driver input/action or the driver-initiated event is still occurring that caused generation of the feedback message or post-event feedback message).

The audio and/or haptic messages may be communicated to the person after the driver controls the racing vehicle. The audio and/or haptic message may be communicated to the person on-demand. In some embodiments, the driver may be able to control the transmission of the audio messages to other people. The audio and/or haptic message may be recalled using a repeat message function. The messaging apparatus 100 may include a user interface 110 for selecting the type of audio messages to be conveyed to the driver.

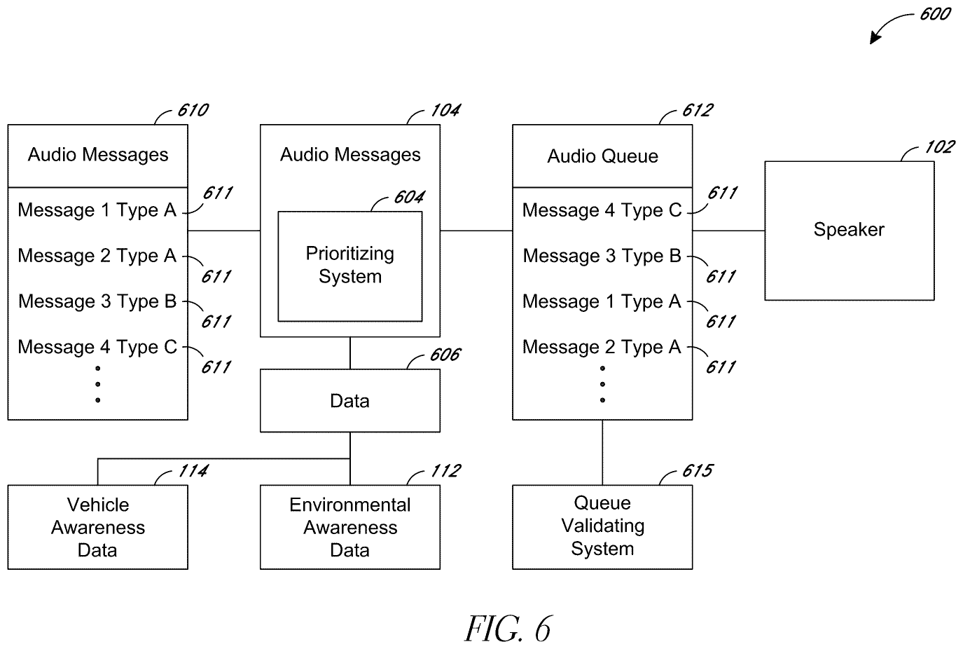

FIG. 6 illustrates an example process 600 for prioritizing audio messages, which can conveyed or communicated to a user. The controller 104 may execute computer-executable instructions for the prioritizing system 604 to function as discussed herein. The controller 104 can cause the system to communicate one or more messages as discussed in reference to FIG. 6 herein. The one or more components described in reference to FIG. 6 can be modules of the controller 104.

The prioritizing system 604 can receive a queue of audio messages 610. The audio messages may be generated by a system similar to audio modifier 302. Each of the audio messages 611 may have content and a type. The prioritizing system 604 may also receive data 606. The data 606 may include environmental awareness data 112. The data 606 may also include vehicle awareness data or vehicle information 114. The data 606 may come from the external data source 108, vehicles sensors 106, or from the CAN bus 105. The prioritizing system 604 may use both the data 606 and the audio message input queue 610 to generate and output audio queue. The prioritizing system 604 may use pre-established or predetermined priorities to generate and output audio queue. The output audio queue may be the play queue fed into the one or more speakers 102 for delivery to the recipient. At each time step, the output audio queue may be accessed by the prioritizing system 604 to update the output queue 612. In some embodiments, the audio messages 611 may be queued based on their respective types. In some embodiments, the queued audio messages may be stored as pointers to audio files. The queue audio files may be stored as pointers to data for generating audio files. The actual output to the one or more speakers 102 may generated on just in time basis or in real time. The actual output to the one or more speakers 102 may be generated in advance of being needed. In some embodiments, multiple audio messages may be conveyed simultaneously. The type of messages that can be played together, and the maximum number of simultaneous messages may be controllable by a user.

The controller 104 can be configured to adaptively modify a selection of audio messages conveyed to the driver based on performance data collected by the controller 104. The controller 104 can also be configured to prioritize audio messages for audio delivery to the driver based at least partly on message type. In some embodiments, the controller is configured to prioritize the audio messages in response to external data 108 received by the vehicle, the external data comprising information corresponding to an environment of the racing vehicle—such as environmental awareness data, including location of the vehicle on the track. The audio messages may be prioritized based on a variety of considerations such as urgency, or settings on the controller 104. The data 606 used by the prioritizing system 604 may include any of the data gathered, received, or transmitted by the messaging apparatus 100. The data 606 may include user preferences. Any of the data 606 may be used to at least partly or include detail to determine how messages should be prioritized. The controller 104 may be configured to prioritize audio messages in response to external data received by the vehicle. The external data may relate to information corresponding to an environment of the racing vehicle.

The controller 104 may use a queue validating system 615. The queue validating system 615 may evaluate the messages 611 in the audio queue 612. The queue validating system 615 may delete one or more messages from the queue if the relevance of those messages has diminished sufficiently as discussed herein. The block 615 may be a time-out system that deletes messages that have been in the audio queue 612 for too long. The queue validating system 615 can discard messages when their relevance diminishes, or when another event supersedes the message. For example, a gear shift message may be delayed due to another message. Subsequently, the gear shift message may be discarded after some period of time, or when a gear shift occurs—rendering the message superfluous. Accordingly, whether one or more messages are communicated by the controller 104 may depend at least partially on what other messages are in the audio que 612. The delayed messages or feedback may be discarded, where the context for the feedback to the driver or operator may be no longer relevant. This condition of discarding a delayed message may be dependent on the accumulated time since the initiating event (e.g., a predetermined time has passed), type of audible metaphor, delivery of subsequent audible metaphors having more context (e.g., based on location), or number or type of other priority communications or messages during the delayed period. In some embodiments, the queue validating system 615 may delete messages from the queue based on user input, data 606, vehicle awareness data 114, and/or environmental awareness data 112. The controller 104 may use the que validating system 615 to not generate certain audio messages if the relevance of the messages no longer applies for a particular operator maneuver or scenario, including the particular operator maneuver or scenario being based on location of the racing vehicle on the road or racetrack.

The controller 104 can use one or more message prioritizing systems discussed herein to coordinate when and if audio and/or haptic messages are communicated and/or overlapped. In some embodiments, the controller 104 can cause the system to communicate the braking marker messages as discussed herein as a prioritized message. For example, the controller can cause the system to communicate braking marker messages before communicating other messages that may be in the que for communication. In some embodiments, the controller 104 can prioritize braking marker messages as a priority message (to be communicated before other messages) based on analysis of vehicle awareness data 114 and/or environmental awareness data 112. For example, the controller 104 may prioritize braking marker messages at predetermined locations on the track to provide messages that allow for proper control of the vehicle in order for the driver to be able to perform other desired operational driver inputs at safe and effective speeds. Accordingly, the braking marker message may be prioritized over other messages in the que due to the inherent dependency of achieving proper driver input based on the braking marker in order for the driver to be able to perform other vehicle operational inputs safely and effectively (e.g., steering into, around, and out of a corner at a safe speed while achieving a relatively fast time through the corner). The predetermined locations on the track for prioritizing braking marker messages may be at predetermined one or more corners on the track.

In some embodiments, the controller 104 can select one or more audio and/or haptic metaphors from a list of messages such in the audio queue 612. The list of messages can include multiple audio metaphors for a single driving event (e.g. entering a corner or turn as discussed herein). The controller 104 may convey an audio and/or message to the driver based on the selected audio and/or message. The controller 104 may select the audio and/or message based on vehicle awareness data 114 and/or environmental awareness data 112 as discussed herein.

As another example, in some embodiments, the controller 104 can prioritize fuel consumption per lap messages to either be communicated or not be communicated based on, for example, the particular message being no longer relevant (such as excess fuel or under pace car control) or of being lower priority in comparison to other messages (e.g., braking marker messages). Accordingly, the controller 104 can determine priority of fuel consumption per lap messages based on vehicle awareness data 114 and/or environmental awareness data 112.

As another example, the controller 104 can cause system to communicate wind direction and force via audio and/or haptic messages to the driver at pre-defined or predetermined corners where such wind data to the driver is more relevant or of higher priority. Audio and/or haptic messages corresponding to wind data at corners may facilitate the driver's ability to properly achieve, for example, one or more corner apex targets as discussed herein by for example, modifying steering and/or braking inputs based on wind force and/or direction. The pre-defined corners may be entered by one or more users. The controller 104 can determine when to communicate audio and/or haptic messages corresponding to the wind direction and force to the driver, and thereby provide the wind direction and force messages to the driver as prescribed or programmed at the time of configuring the controller 104.

Providing the driver with feedback in a real time format as discussed herein can be beneficial, but the human driver has limited attention, and it can be important not to overload the driver or other persons with messages. As part of situational awareness/functionality and prioritization, the controller can determine which messages are more important in one position along the track but not as important at another position along the track, including based on other environmental and/or vehicle awareness data 112, 114. The controller 104 can be determine using various data the priority and importance of a message to selectively communicate the audio and/or haptic message based on, for example, priority and situational awareness.

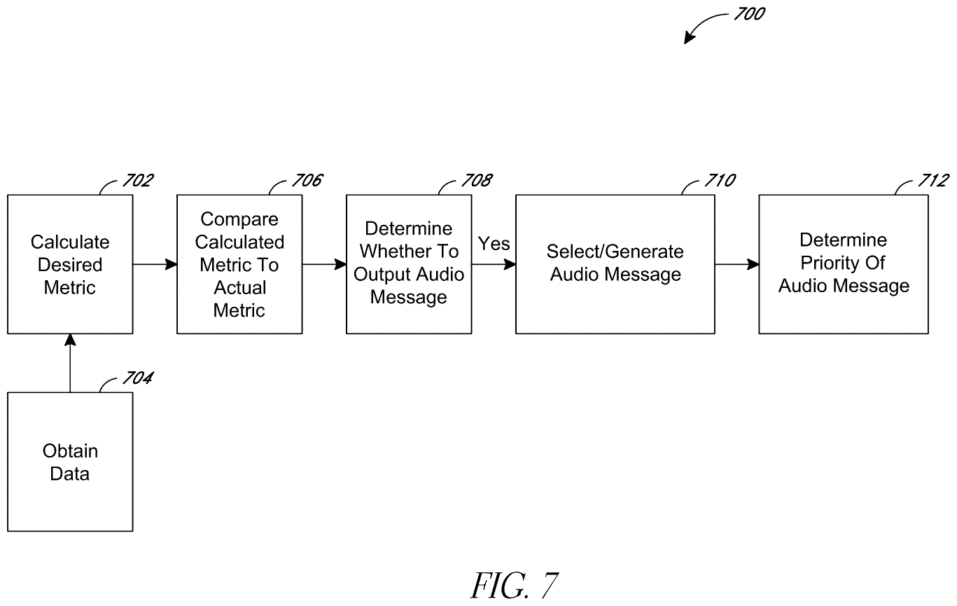

FIG. 7 illustrates an example embodiment of a process for generating audio messages. FIG. 7 illustrates an example metric calculation process 700. Process 700 may use the processor 126. FIG. 7 can be an example embodiment of a process for providing audio feedback to an operator or driver based on comparing an actual performance metric to a desired performance metric. At block 702, the system calculates the desired metric related to a driver action. This calculation may be based on data obtained at block 704. The block 704 data may include vehicle data and environmental data. At block 706, the system compares the calculated desired metric to the actual metric achieved by the driver. At block 708, the system decides whether to output an audio message. If the system determines that an audio message should be generated it proceeds to block 710. At block 710, the system selects and/or generates an audio message. The process for selection/generation of the audio message may be similar to audio modifier 302. After the message is generated, the priority of the audio message is determined at block 712. At Block 712, the system may utilize the prioritizing system 604. The metric could be timing, a vehicle dynamics metric, a telemetry metric, and/or another metric as discussed herein, including metrics such as steering, braking, accelerating, regenerative braking, energy harvesting, deployment of energy, etc.

In some embodiments, the messaging apparatus 100 is configured as a system for improving racing vehicle driver performance. The system may include a speaker 102 capable of communicating audio signals to a driver. The speaker 102 may be controlled by a controller 104, enabling the speaker to communicate performance-related information to the driver. To do so, the controller 104 may receive vehicle information, such as from an external data source 108 or from the vehicle via, for example, the CAN bus 105.

The controller 104 may be further configured to use the vehicle information to communicate to the driver an audio message that notifies the driver of a desired metric related to performing a driver action. The desired metric may be calculated, such as block 702, or may be input by a user. The driver action may correspond to controlling the racing vehicle. The desired metric may depend at least partly on a weight of the racing vehicle distributed on a particular wheel of the racing vehicle. The desired metric may be based, at least partly, on a desired tire slip relative to a surface of travel of a tire of the racing vehicle on a racetrack. The determination of the desired tire slip may be based, at least partly, on spin of the tire relative to the surface of travel on the racetrack. In some embodiments, an audio message will notify the driver of a transitional input ramp associated with steering the racing vehicle to achieve the desired metric.

In some embodiments, the controller 140 can calculate the desired metric based at least partly on the vehicle information collected by the vehicle sensors. The controller may further compare the desired metric to an actual measured or calculated metric of the driver action and determine whether to output the audio message based on a result of the comparison. Block 706 may include this comparison. If an audio message is to be output, the controller 104 may select the audio message to convey to the driver based on a result of the comparison. In some embodiments, the controller 104 may be configured to determine the desired metric at least by comparing a vehicle location to a predetermined location on a racetrack. The desired metric may correspond to a maximum of at least one of acceleration, deceleration, or cornering of the racing vehicle. These maximums may be determined at least partly based on a reference location on a racetrack. The controller 104 may be configured to determine the desired metric based on any of the data collected by the sensors on the racing vehicle. The controller 104 may be configured to determine the desired metric based on any of the data received by the racing vehicle from an external source. The controller 104 may send data to a system, such as a server 134, located external to the vehicle for analysis. The controller may receive data from the system, such as the server 134, related to the desired metric, or other parameters.

In some embodiments, the system includes a second speaker 102 capable of communicating audio signals to a person. The audio signals may be configured to notify the person of the desired metric related to performing the driver action. Performing the driver action may correspond to controlling the racing vehicle.

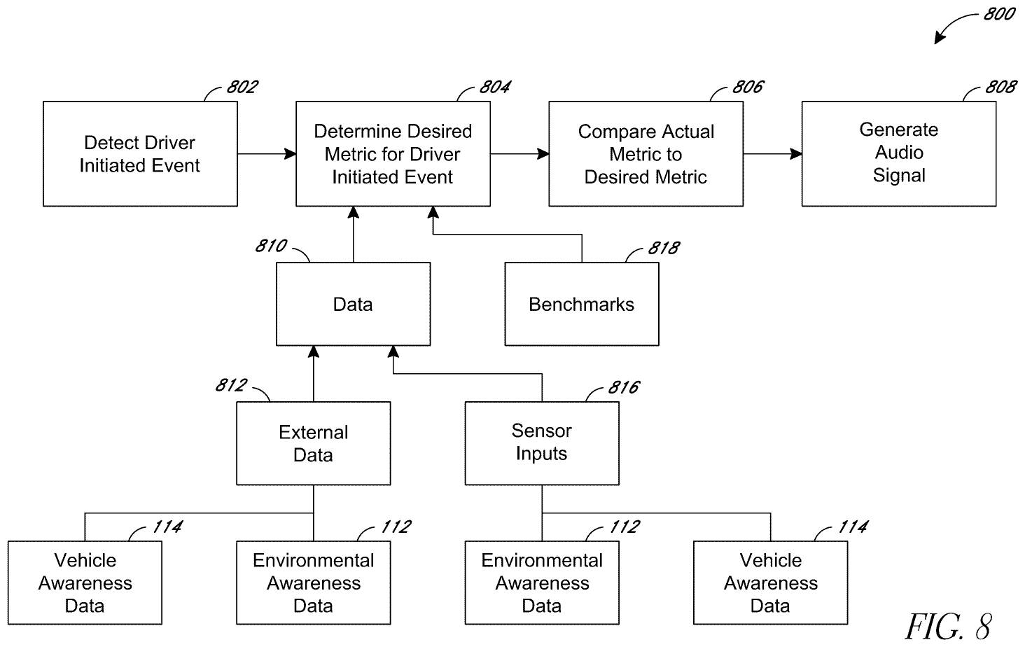

FIG. 8 illustrates an example embodiment of a process for generating audio messages, including with certain inputs. FIG. 8 can illustrate a desired action calculation process 800. FIG. 8 can be an example embodiment of a process for providing audio feedback to an operator based on comparing an actual performance metric to a desired performance metric, the performance metric calculated based on data. As discussed herein, performance metric can correspond to one or more desired or predetermined thresholds or ranges. Process 800 may be implemented by the processor 126. At block 802, the system detects a driver-initiated event. Block 804 determines the desired metric for the driver-initiated event. The desired metric is determined based on data 810 and benchmarks 818. The benchmarks 818 may be entered by a user at an earlier time, or they may be adaptively generated by the system in response to driver and vehicle performance. The data 810 may include external data 812 that is obtained from a data source external to the vehicle or from sensors 106 located on the vehicle. Both the external inputs/data 812 and the sensor inputs/data 816 may include environmental awareness data 112 and/or vehicle awareness data 114. The system compares the actual metric to the computed desired metric at block 806. Based on the comparison of the actual metric and the desired metric, the system generates an audio signal at block 808. The audio signal may be generated in a manner consistent with the audio modifier 302. The benchmarks 818 may include metrics based on position of the vehicle, or performance parameters of the vehicle or the driver. The metric could be timing, a vehicle dynamics metric, a telemetry metric, and/or another metric, including metrics such as steering, braking, accelerating, regenerative braking, energy harvesting, deployment of energy, etc.

In some embodiments, the desired metric related to performing a driver action is based at least partly on at least one of a position or a change in position of a nearby racing vehicle. This information may be conveyed to the system as external data 812 or sensor inputs 816. The positions of nearby racing vehicles may be determined using vehicle sensors 106 configured to detect the position and the change in position of the nearby racing vehicle during movement of the racing vehicle. In some embodiments, the position of the nearby racing vehicle may be known via data received an external data source 108, such as from a Global Positioning System (GPS) configured to track the position and the change in position of the nearby racing vehicle. In some embodiments, the position of nearby racing vehicles may be known via vehicle image, LIDAR, radar, and/or proximity sensors. In embodiments, image, LIDAR, radar, and/or proximity data may be received by the system from external sources and/or sensors as external data 108. The controller 104 is configured to receive information related to the nearby racing vehicle based at least partly on at least one of data received from the vehicle sensors or from the GPS (directly or indirectly). Other equipment may also be used to generate data regarding the position of the nearby racing vehicle.

In some embodiments, the controller 104 can determine whether a driver action was performed early or late relative to a desired metric. In some cases, this includes comparing the racing vehicle location at the time of the action to a pre-defined location on a racetrack. Location information may be represented by block 810 and combined with benchmark location data as represented by block 818. This information may be used to determine the desired metric and compare it to the actual metric of a driver-initiated action at blocks 804 and 806. The controller may be configured to output an audio signal in response to determining that the actual metric related to driver performance is greater by more than a threshold amount as compared to desired metric. The controller may be configured to output the audio signal in response to determining that the measure metric associated with driver performance is less by more than a threshold amount, as compared to the desired metric. The audio signal may vary in proportion, or by another function, to the variation between the desired and actual metric.

The desired metric related to performing the driver action is based at least partly on at least one of a position or a change in position of a nearby racing vehicle. The controller 104 can receive information related to the nearby racing vehicle based at least partly on data received from the vehicle sensors 106. The vehicle sensors 106 can be configured to detect the position and the change in position of the nearby racing vehicle during movement of the racing vehicle. The location of the other racing vehicle may also be determined, based at least partly on data received from a Global Positioning System (GPS) configured to track the position and the change in position of the nearby racing vehicle. Each of these data sources could be used independently, or they could be relied on together.

In some embodiments the messaging apparatus 100 is configured for improving racing vehicle driver performance. A method to improve driver performance using the messaging apparatus may involve detecting a driver-initiated event performed during driving of a racing vehicle such as at block 802. This detecting may be performed by a computing device that monitors real-time vehicle information associated with the racing vehicle. The computing device could be a server 134, a processor 126, or a computing device connected to other components of the vehicle, such as a computing device integrated or connected to the vehicle sensors 106. The method may further include determining a desired metric related to a driver-initiated event based at least partly on collected sensor data. Sensor inputs 816 and external data 812 may both be used to determine the desired metric related to the driver-initiated event. Other data may also be used, such as data input by the driver, any data generated by analyzing sensor inputs 816 or external data 812, or any other data generated by or sent to the vehicle or the server 134. The driver-initiated event may be a driver input to the vehicle via the vehicle controls. The method may then include comparing actual metric related to the driver-initiated event to the desired metric. Depending on the outcome of this comparison an audio signal may be delivered to the driver. The messaging apparatus 100 may deliver any generated audio message via a speaker 102. The contents of the audio signal may depend on the result of the comparison. The audio signal may be played in real-time. The audio signal may be played to the driver sometime after the driver-initiated event. The audio signal may correspond to a degree to which the actual metric related to the driver-initiated event varied from the desired metric. The audio signal may be changed via a step function by the comparison.

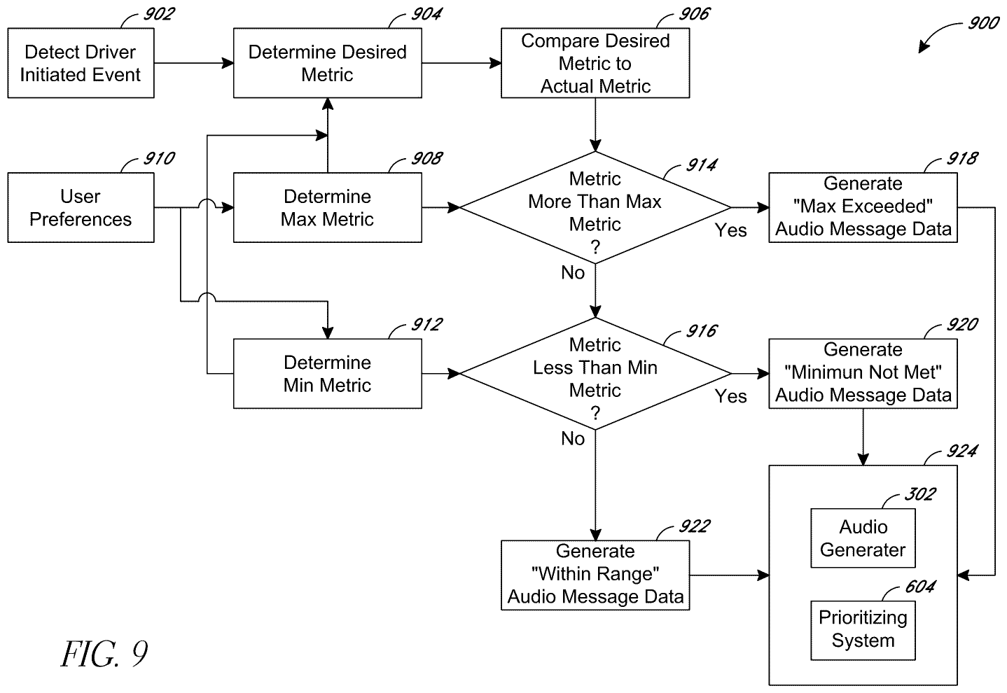

FIG. 9 illustrates an example embodiment of a process for generating audio messages. FIG. 9 illustrates an example embodiment of a process for notifying the driver when certain performance metrics are above a maximum metric or below a minimum metric. FIG. 9 illustrates a process 900 that may be implemented by the controller 104 to notify the driver when a metric associated with driver actions are below a predetermined low threshold or above a predetermined high threshold (e.g., a predetermined range). Like other processes, this process may, for example, be implemented in software or firmware executed by the controller's processor 126. At block 902, the process may detect a driver-initiated event such as a shifting event, braking event, or acceleration event. The driver-initiated event could be any event comprising an input from the driver. In some embodiments, the system could be configured with specific rules for identifying certain driver-initiated events. These specific rules could classify complex combinations of basic driver inputs. This detection may be accomplished by the processor 126 receiving data from the vehicle sensors 106, the CAN bus 105 or the external data source 108. For example, the data can be car sensor signal data, or equivalently vehicle sensor information, collected through the CAN bus. The data can include GPS and/or beacon data. The data can also include external sources data, such as, for example, a message from another team member. At block 904, the system determines a desired metric for the driver-initiated event. In some embodiments the desired metric for the driver action may be calculated from various metric datum points. For example, the metric could be calculated from the start of a race, from the start of a lap, from the start of a corner, or from the start of any position as selected by the user, or the metric datum may be defined based on detecting selected driver actions. In some embodiments, the metric datum may be configurable by the user. In some embodiments, datum triggers may be selectable by the user. In some embodiments, user inputs can be received through the user interface 110 to select the metric datum. The metric could be timing, position, a vehicle dynamics metric, a telemetry metric, and/or another metric, including metrics such as steering, braking, accelerating, regenerative braking, energy harvesting, deployment of energy, etc.

In some embodiments, the appropriate position datum may be determined using adaptive techniques or machine learning. In some embodiments the desired position for the driver action may be calculated from various position datum points. For example, the position could be calculated from a static position, from the vehicle starting position at the start of a lap, from the start of a corner, or from the start of any position as selected by the user, or the position datum may be defined based on detecting selected driver actions. In some embodiments, the position datum may be configurable by the user. In some embodiments, datum triggers may be selectable by the user. In some embodiments, user inputs can be received through the user interface 110 to select the position datum. In some embodiments, the appropriate position datum may be determined using adaptive techniques or machine learning.

In some embodiments, the process 900 may determine multiple metrics for the driver's action at block 904. In some embodiments, the system may determine one or the other. In some embodiments, the system may determine one or the other based on the various data received by the system. Processes 700 and 800 may similarly be applied to single or multiple metrics as described with respect to process 900 above.

The desired metric may be calculated by the controller 104 prior to the event (in which case it may be the basis for an audio signal applied to the driver), or during or after the event. As shown in block 904 and 906, the controller 104 may alternatively compare the vehicle's actual location to the determined desired location.

At block 906, the system compares the desired metric(s) of the driver-initiated event to the measured metric(s) of the actual driver-initiated event.

At blocks 914 and 918, if the metric exceed a maximum threshold metric level, the controller 104 generates “max exceeded” audio message data to notify the driver that the action resulted in a metric exceeding a maximum for that metric. At block 916 and 920, if the metric was less than a minimum threshold metric level, the controller 104 generates “minimum not met” audio message data to notify the driver that the action resulted in a metric that was less than a minimum for that metric. At blocks 922 and 924, if the metric resulting from the driver action is not below a threshold minimum metric level and is not above a maximum threshold level, the controller 104 generates “within range” audio message data to notify the driver that the action was performed within the desired range via, for example, the audio generator 302 and/or prioritizing system 604 as discussed herein. At blocks 908 and 912, the process 900 determines the maximum, or desired maximum, threshold metric level and minimum, or desired minimum, threshold metric level respectively. The max/desired max threshold metric level and min/desired min threshold metric level may be based at least partly on user preferences 910. The determined desired metric level from block 904 may also be an input to the max/desired max threshold metric level 908 and min/desired min threshold metric level 912 determinations. In some embodiments, the max/desired max threshold metric level and min/desired min threshold metric level determinations 908 and 912 may use any of the data conveyed or gathered by the controller 104.

The threshold or thresholds determined in blocks 908 and 912 may be programmable by the driver or driver's team via a user interface of the controller 104. The controller's user interface may also enable the driver or driver team to enable and disable various types of feedback signals.

The audio message data generated in blocks 918, 920, and 922 can be used to generate audio messages. In some embodiments the audio message data generated in blocks 918, 920, and 922 can be used to generate and convey audio messages to a user at block 924. In some embodiments, the audio message data from blocks 918, 920, and 922 is sent to the audio modifier module 302 and/or the prioritizing system 604.

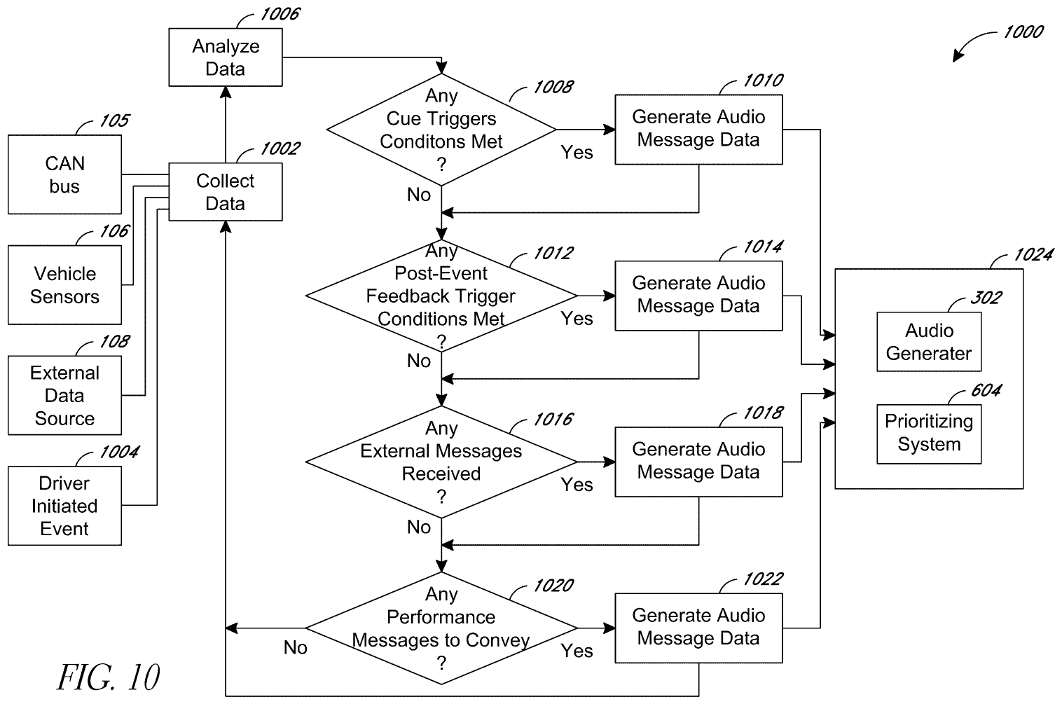

FIG. 10 illustrates an example embodiment of a process for determining what audio message data to generate. FIG. 10 illustrates a process 1000 that may be implemented by the controller 104 to determine what audio message data to generate. At block 1002, the system receives data from a variety of sources, the variety of sources may include the CAN bus 105, the vehicle sensors 106, the external data source 108, and the driver-initiated events 1004. The driver-initiated event data 1004 may be conveyed from one of the other sources, such as by the CAN bus 105 or the vehicle sensors 106. At block 1002 and/or block 1006, the controller 104 may log data collected and detected from the sensor signals corresponding to events associated with vehicle information, execution information, and/or auxiliary information as described herein, such as, for example, braking, acceleration, shifting, wheel spin, etc.

At block 1006, the controller 104 analyzes the collected data to determine appropriate action at decision points 1008, 1012, 1016, and/or 1020.

At decision point 1008, the controller 104 determines whether any cue trigger conditions are met. Cue trigger events can include information relating to real time performance, such as, for example, timing of braking, accelerating, shifting, and cornering, wheel slip, speed, wheel angle, and/or other cue events as described herein.

When a cue trigger condition is met, the controller 104, at block 1010, can generate audio message data. The audio message data can be sent to block 1024, wherein the audio message is generated via, for example, the audio generator 302 and/or prioritizing system 604 as discussed herein. The cue trigger event can be logged in the memory 128 of the controller 104. The cue trigger conditions can be determined based on current sensor signal data, including what the driver is doing in real time, and logged events. For example, when the controller 104, based on current sensor signal data, determines that the driver should be braking, but the driver has already braked based on logged event data, the controller 104 can proceed to decision point 1012 and determine whether to generate post-event feedback audio data as described herein such as, for example, to let the driver know that the braking was performed too early. As another example, when the controller 104, based on current sensor signal data, determines that the driver should be increasing (or decreasing) acceleration, the controller 104 can determine whether to increase (or decrease) the output of the acceleration audio signal based on the previously generated and logged intensity of acceleration audio signal at block 1010. Described differently, as the controller 104 generates and logs acceleration audio message data, the controller 104 then determines at decision point 1008 whether to increase (or decrease) the intensity of the acceleration audio message data based on current sensor data and the logged intensity of the previous or still continuous acceleration audio message data.

After block 1010, or when a cue trigger condition is not met at decision point 1008, the controller 104 can determine whether a post-event feedback condition is met at decision point 1012. Post-event feedback conditions can include post-event feedback as discussed herein. When a post-event feedback condition is met, the controller 104, at block 1014, can generate audio message data. The feedback event can be logged in the memory 128 of the controller 104. The controller 104 can use logged post-event feedback and current sensor data to determine, for example, whether to generate audio message data if the event is continually occurring. When the controller 104 determines that it has already generated post-event feedback based on current sensor data, the controller 104 can instead generate an audio cue message to change driver behavior. Accordingly, the controller 104 can use current sensor data, logged or queued audio message data at block 1010, and/or logged post-event feedback data at block 1014 to determine what type of audio message data to generate. For example, based on a logged audio message data event at block 1010, the controller 104 can change the audio message data to be a post-event feedback signal after determination that the audio message associated with the cue signal has already been generated. In some embodiments, if the system detects that the time window wherein a cue message to the driver is useful, has elapsed, the system may delete the audio cue message data or convert the audio cue message data to feedback event audio message data. The time window corresponds to the time before the driver-initiated event. Once the driver-initiated event has been executed, if the cue message has not yet been conveyed to the driver, the cue message data associated with the cue message should be deleted or converted.

After block 1014, or when a post-event feedback condition is not met at decision point 1012, the controller 104 can determine if a message from an external source, such as external data source 108 or server 134, has been received at decision point 1016. Messages can include an audio message data sent by another member of the racing team and as described herein. When a message from an external source has been received, the controller 104, at block 1018, can generate audio message data corresponding to the external message. The external message event can be logged in the memory 128 of the controller 104. The controller 104 can use logged external message events to determine when a repeated external message is being received within a predetermined timeframe and suppress the repeated external message to minimize distractions to the driver.

After block 1018, or when an external message has not been received as determined at decision point 1020, the controller can determine if the vehicle performance data should be used to generate audio message data. For example, the controller 104 may determine whether tire life or fuel levels should be conveyed to the driver. The decision point 1020 decides which continuously generated data, not linked to a specific driver action, or received message, should be conveyed to the driver. The rules for determining which performance levels correspond to generating audio message data can be configured by the user. The rules may use a threshold system, wherein if a certain parameter, such as fuel levels below 25%, exists then audio message data conveying this information should be continuously generated. After block 1022 or 1020 the process 1000 is repeated indefinitely.

The audio message data generated in blocks 1010, 1014, 1018 and/or 1022 can be used to generate audio messages. In some embodiments the audio message data generated in blocks 1010, 1014, 1018 and/or 1022 can be used to generate and convey audio messages to a user at block 1024. In some embodiments, the audio message data from blocks 1010, 1014, 1018 and/or 1022 are sent to the audio modifier module 302 and/or the prioritizing system 604.

The process steps of FIG. 10 do not have to be performed sequentially. The process steps can be carried out in parallel with other steps. The process steps can be carried out by multiple processors in parallel. For example, the controller 104 can be continuously collecting data at block 1002 while performing any of the process steps at blocks 1006 to 1022 such as, for example, while generating audio message data at blocks 1010, 1014, 1018 and/or 1022. Similarly, the controller 104 can be continuously analyzing collected data at block 1006 while performing any of the process steps at blocks 1002 and 1008 to 1022.

All the steps, computations, or programs could be run or executed on the processor 126 mounted to the controller 104. In some embodiments, computational steps, especially those steps discussed with relation to FIGS. 2-8 can be executed by a server 134. If the step is executed by the server 134, the server may communicate the audio files back to the processor 126 to route to the speaker 102. Various distributions of computing activities between the server 134 and the processor 126 are possible. Computing on the server may be useful for improving reliability, speed and/or access to tools such as machine learning. In some embodiments, the prioritizing system 604, audio modifier 302, decision tree 400, decision tree 500, timing calculation 700, and/or desired action calculation 800, may be machine learning systems. When these systems are machine learning systems, they may utilize data sets gathered and stored on the server 134 or the processor 126. The data may include all the data gathered from the vehicle sensors 106, ECMs 107, ECUs 109 and gathered in connection to the external data source 108. The data may be subjected to post-processing. The data may be associated with the driver and vehicle from which it was generated. The data may be associated with race results, or other performance indications. The data may be screened for errors generated by the sensor systems.

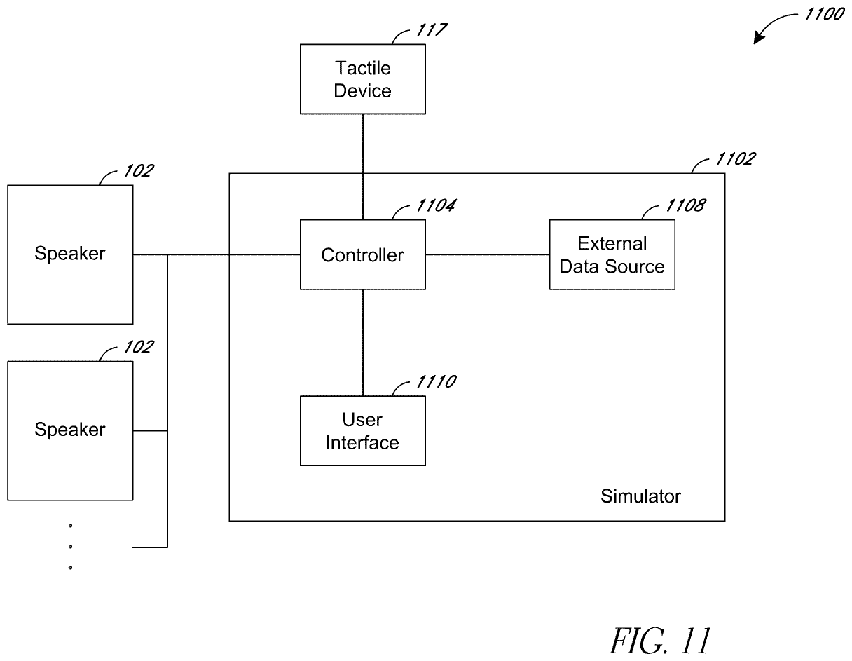

FIG. 11 illustrates an example embodiment of a simulator 1100. FIG. 11 can be an example embodiment of a simulator with a messaging apparatus. The simulator 1100 may be similar to the messaging apparatus 100 with possible various differences while having and utilizing the various audio messaging functions and controls as discussed herein, including message prioritization, simultaneous playback, and/or message generation such as variation in frequency, pitch, beat, volume, tone, rhythm pattern, chords, and/or sequence of chords. The simulator 1100 may include a data source 1108. The data source 1108 can store information, data, and/or program code to simulate driving a vehicle or can store information and/or data from a past driving event to be used by the driver, pit crew, engineer, and/or team members to review performance of the driving event using audio and/or haptic messages as discussed herein, correlating past driver events and actions to recorded audio and/or haptic messages to review driver performance from the past driving event without or without simulating a racing vehicle. The data source 1108 may be connected to the controller 1104 via a network connection or a wired connection. The controller 1104 generates audio messages (and/or tactile messages in some embodiments) that are played through the one or more speakers 102 (and/or conveyed through one or tactile devices 117) as discussed herein. The simulator 1100 may use inputs to control a simulated vehicle machine, or equipment control. Audio metaphors can be generated based on the simulated vehicle machine, or equipment control and simulated vehicle machine, or equipment control environment as discussed herein.

In some embodiments, the simulator 1100 may use data gathered from a real race to generate simulated audio signals. In some embodiments, the simulator may be used to enable users to test audio metaphor generating schemes based on real race and/or event data or simulated race and/or event data.

In some embodiments, the simulator 1100 can replay an audio message stream substantially or generally, including exactly, as it was played to a driver or operator of the vehicle, machine, or equipment control. In some embodiments, the simulator 1100 can generate various audio messages based on data gathered during a race or other events during operation of vehicle, machine, or equipment control.

The simulator 1100 may be useful for E-Sports, driver training, and audio metaphor development for, for example, training, feedback, adjustment, and/or advancement of audio metaphor application.

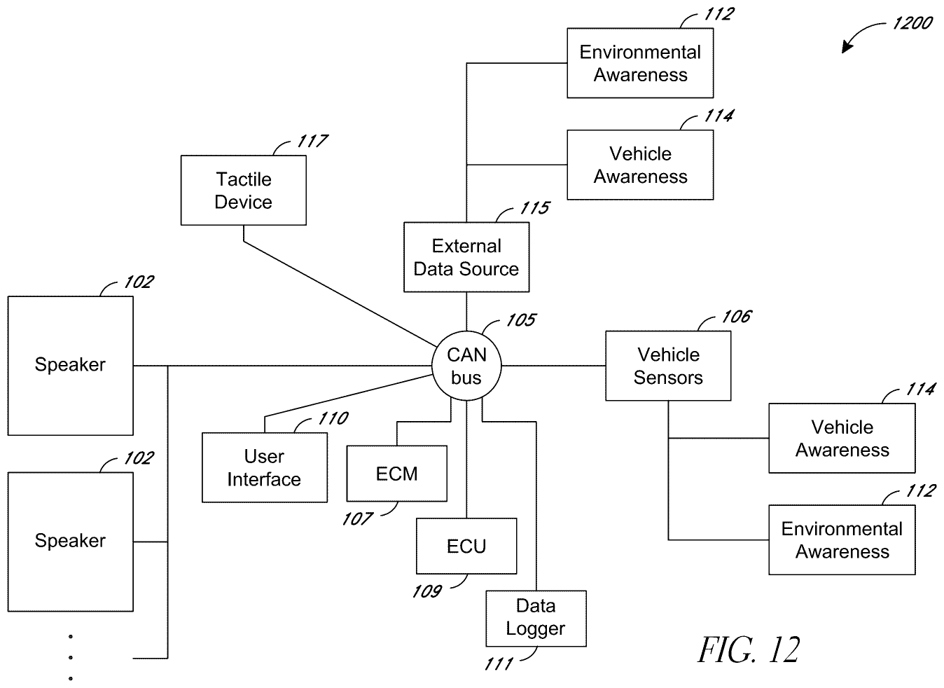

FIG. 12 illustrates an example embodiment of a messaging apparatus connected to a vehicle a CAN bus of a vehicle. FIG. 12 illustrates an example embodiment of a messaging apparatus 1200. The messaging apparatus 1200 is similar to the messaging apparatus 100 with various differences as described herein. The ECU 109 in the messaging apparatus 1200 can substantially conduct the functions served by controller 104 in messaging apparatus 100. In some embodiments, the one or more speakers 102, user interface 110, and tactile device 117 are connected to the CAN bus 105. The ECU 109 may implement the program code 130. Implementation of the messaging apparatus 1200 may be reflective of a TAG-320/integrated street vehicle implementation. Implementing the messaging apparatus 1200 as discussed herein may be achieved without having an additional controller external to the CAN bus 105.

### Sophisticated Audio Metaphors for Operator Feedback Control

The messaging apparatus 100 may be configured to receive inputs from a vehicle sensor or other sensor and generate audio metaphors. An example embodiment of a system for generating audio metaphors is shown in FIG. 3. In some embodiments the audio messages 304 can be audio metaphors. Audio metaphors and audio messages may be used interchangeably. The audio metaphors may be generated according to rules. The rules used to generate the audio metaphors can be configured by a user. In some embodiments a touch screen or other input device, such as user interface 110 can be used to configure the rules. Operators, drivers, and other recipients of the audio metaphors may understand the metaphors and be able to learn about the sensor data by hearing the audio metaphor. The metaphors communicate data in real time as an audible expression, message, signal, cue, or otherwise. In the case of vehicles, the metaphors communicate vehicle information in real time to drivers, passengers, or pit-crews. Recorded telemetry data can be used in a simulated playback, to allow non-real-time post-analysis. Post-analysis may include playing audio metaphors as they occurred at the time of the recording. The audio metaphors may be suitable for use in connection with machines other than vehicles, such as utility scale power plants.

The messaging apparatus 100 and associated controller 104 can enhance the ability of a driver, especially a racing vehicle driver, to understand the vehicle dynamics of their racing vehicle. In particular, the real-time feedback loop between control input and change in vehicle dynamics, as communicated by changes in audio metaphors, can be effective to enable the driver to tune their control inputs to improve vehicle and driver performance.

Additionally, the audio metaphors can include information gathered by sensors that is related to the environment of the sensors and the car. In the case of racing vehicle mounted sensors, such as vehicle sensors 106, this could include data about the environment of the racing vehicle. Information about the environment of the racing vehicle could include the location of other racing vehicles, or obstacles. Data can also come from a source external to the race car such as external data source 108.

The sensor data, and related audio metaphor, can be recorded. This may be useful for training purposes. Recorded sensor data can be used to generate sound metaphors on-demand. The generated sound metaphors can use any sound metaphor generation configuration as input by a user.

The messaging apparatus 100 generates audio messages, and audio signals in response to data received from the monitored vehicle. In some embodiments, the controller 104 receives vehicle data from a CAN bus 105, directly from vehicle sensors 106, or from an external data source. The received data is used to generate a sound metaphor. The sound metaphor may be sent to the user in response to various conditions. Some of the conditions are described above. An audio signal, audio message, or audio cue may all comprise a sound metaphor.

The controller 104 may dynamically generate the sound metaphor in response to the continuing variation in vehicle dynamics and the environment. The information in the metaphor is conveyed to the driver, or other person. The driver can use the metaphor to adjust their input controls according to the tones heard in the metaphor. For example, the metaphor may be a non-harmonious metaphor when an operator is operating a vehicle sub-optimally, or outside of an operating range. The operating range may be pre-computed, computed on the fly, computed by an external device, and transmitted to the vehicle, or otherwise determined. As the driver brings the operation closer to the optimal point, or range, the metaphor may become increasingly harmonious. Hence, the metaphor can guide the driver towards an optimum and/or desired. As the driver guides the metaphor towards harmony, the driver simultaneously drives the vehicle more optimally. The driver inputs influence how the vehicle dynamics, and hence the sound metaphor, change over time.

The sound metaphor may be consistently or continuously conveyed to the driver or conveyed at time intervals. The time intervals could be evenly spaced on unevenly spaced. The metaphors may be conveyed in response to specific action or events. The sound metaphor is conveyed using the speakers 102.

The sound metaphor may simultaneously convey information about various sensor systems, vehicle operations, vehicle dynamics, vehicle environment, or related topics. For example, the sound metaphor may convey information about the location of nearby racing vehicles, about the current variation from the optimal wheel slip ratio and fuel levels and/or usage rates all simultaneously. In some embodiments, the sound metaphor may relate to a single category of sensor data at a time. The data being conveyed via the sound metaphor may change based on an interval, a user input, a pre-programmed timing, a pre-programmed basis using location of the vehicle, performance conditions of the vehicle, performance of the operator versus optimal operator performance, the environment of the racing vehicle—such as proximity of other racing vehicles, or other basis.

In some situations, it may be needed or desirable to enter a significant and abrupt change in the audio metaphor to illicit an immediate reaction in the driver. For example, the driver may be driving through a corner while a sound metaphor conveys information related to achieving an optimal wheel slip ratio. However, if during this maneuver, an elevated risk of loss of rear wheel traction occurs, the sound metaphor may suddenly change to alert the driver of this condition. The sudden change in the sound metaphor in response to the condition change may be based at least partly on the configuration of the priority system. This abrupt change in the audio metaphor is useful to illicit an immediate driver reaction to correct the condition. The nature and intensity of the variation may vary in proportion to the immediacy of the action to be performed. Generally, the audio metaphors may have various parameters which may vary proportionally to various vehicle dynamic indications as conveyed by the vehicle's sensors. In some embodiments, the parameters of the audio metaphors may have a non-proportional variation related to various vehicle dynamic indications.

The metaphors conveyed to the users may convey different information depending on the relative importance of information given the current situation of the vehicle. The situation of the vehicle may include the performance and location of the vehicle, and environmental considerations.

The messaging apparatus 100 has a controller 104 that is configured to emphasize and/or prioritize sounds based on present vehicle dynamics and environment. The system can be configured to determine the information with highest priority to the listener. The prioritizing system 604 may generate the audio queue for conveyance to the users. The prioritizing system may generate various different audio queues for different users. In some embodiments, the system will queue up messages and play those messages which have highest priority first. In some embodiments, the system may increase the intensity of the highest priority messages while simultaneously playing the other messages. In some embodiments, the system may multiplex the audio metaphors together (e.g., simultaneously deliver multiple audio metaphors corresponding to various operator control inputs) and increase the intensity of the highest priority messages while simultaneously playing the other metaphors. Simultaneous or multiplexed messages may be prioritized as discussed herein to determine which audio metaphors should increase in intensity and be more prominent by, for example, being played louder relative to other messages either intermittently or continuously.

For example, contamination on the road presents a safety hazard. The hazard may be identified, either by an onboard sensor, or by data transmitted from outside the vehicle to the vehicle. The controller 104 can receive this data and determine how relevant the data is at the present time. With the relevance determined the system may determine where in the priority queue to locate the new message regarding the road contamination.

In the case where the contamination is distant from the car, the priority of the contamination message can be low. In contrast, the relevance of the road contamination can be high, for instance when the vehicle is close to the contamination. When the priority of the road contamination is high, the audio metaphor associated with the road contamination may be prioritized and moved up in the queue. The system may react to this differently depending on how it is configured. In some configurations, the controller 104 may replace the current audio metaphor with a new metaphor relating information about the road contamination to the driver.

In some configurations, the controller 104 can mix the audio metaphor relating to the road contamination into the existing metaphor. The metaphor mixing may take numerous forms. For example, mixing in the new high relevance information into the audio metaphor may include playing a new metaphor contemporaneously with the already playing metaphor or metaphors. In this case, the change may include adjusting the relative volume of the different metaphors. The objective of the modulation of the audio cue or message is done to convey the new high priority information to the driver or other listener. In some embodiments, the system may determine to adjust the audio metaphor or replace the audio metaphor with new priority information based on vehicle dynamics, driver's input, and changes with respect to the environment. In some embodiments, adaptive audio volume is used. Adaptive audio volume may include adjusting a volume of an audio metaphor based on ambient conditions/environment, road speed, user preferences, message priority, and/or other factors.

The priority function, including both the replacement of current messages, and mixing of new, priority, messages are described above with respect to a new condition of a contaminated road, however this concept can be applied to any new sensor input that is ranked with a high priority. The priority may be based on message type. In some embodiments, machine learning trained on data related to race and/or practice outcomes and data gathered relating to racing vehicles may be used to determine what priority should be used.

The audio metaphors can be generated with regard to the effect of various tones on human emotional state. The musical metaphors may be configured to produce intuitive sensations of affirmation, optimism, success, calmness, discomfort, pain, fear, anxiety, risk et al. For example, in some motorsports it may be desirable to cut a corner to decrease lap time. This process may, however, generate an impact force on the car as the as the vehicle strikes the corner. A vehicle may be able to tolerate some degree of impact (curb strike force). However, above some threshold the vehicle may begin to suffer damage. Repeated or very high strike forces can lead to mechanical failure. An audio metaphor can be used to convey information regarding curb strike force to the driver. The sound metaphor may have a varied expression depending on the nature and magnitude of the curb strike impact. In the case of a minimal curb strike, the sound metaphor may emphasize encouragement. It may be possible to determine the optimal curb strike force. The optimal curb strike force may be determined as the curb strike force that is unlikely to cause mechanical damage during the remaining course of the race if the curb strike were repeated at all corners and that minimizes the lap time. The closer the curb strike force is to the optimal curb strike force the more encouraging the sound metaphor may be. The sound metaphor may indicate degrees of success. An unacceptable impact force could result in the conveyance of a sound metaphor that sounds painful.

Various emotions may be conveyed by specific sound effects. For example, an emulated cry of pain (“ouch!”) may be used to convey pain. Alternatively, variations in musical instruments may be used. Similarly, a pattern of “clapping hands” may be played to signal success. Extending the example above, if the driver cuts the corner and the car receives an unacceptable curb strike, then the audio metaphor may be an anguished “ouch!” In contrast, if the car strikes the curb with the optimal force, then the audio metaphor may include a “clapping hands” track. In some cases, the audio metaphor associated with the emotion may be played alone. In some embodiments, the emotional metaphor is overlaid on an audio metaphor that is simultaneously presenting other information to the driver, as discussed elsewhere in this disclosure. In some embodiments, the sound to convey an emotion may be a recording of an object, a being, or a situation that commonly elicits and emotional response. For example, children laughing may be correlated with joy, a wolf's howl may be correlated with fear. These correlations can be selected by the user ahead of time.

In some embodiments, the emotional component of the audio metaphor could include respiratory and other bodily sounds. For example, fear may cause an increase in breathing rate and heart rate. The audio metaphor may mimic this response. For example, to convey fear to the user, the audio metaphor may incorporate a soundtrack with a quick heart rate and quick breathing. In some cases, this soundtrack could be reproduced based on readings taken from the actual user at a previous date. For example, the pulse, breathing rate, and/or other physiological conditions of the user could be recorded under various emotional test conditions—like a fearful condition. These emotional data sets could be used to reproduce an emotion laden audio metaphor. The emotional audio metaphor may be overlaid onto another audio metaphor.

The audio metaphors can be played as audio feedback that is temporally displaced from the actual event. This delay may be used to avoid driver sensory overload during high mental load situations, thereby mitigating distraction when needed or desired, and conveying the feedback to the listener when the driver is more receptive to the information. In some cases, the playback may occur as soon as the driver has completed an event. The information can be used to convey both the presence of an event, and/or how successfully a maneuver was conducted.

In some embodiments, the sound or audio metaphor may include a harmony-based metaphor. The harmony-based metaphor may be a dynamically generated audio metaphor which varies based on driving dynamics and the environment of the vehicle. The metaphor may be relatively more non-harmonious, disharmonious, inharmonious, discordant, or dissonant as the car is driven relatively less optimally or further outside an operating range. The audio metaphor may be relatively more harmonious the relatively more optimally the vehicle is controlled or while the vehicle is driven within a target or predetermined operating range. This system is beneficial to allow the driver to tune their control inputs with quick feedback from the audio system. As the driver adjusts their operating inputs to optimize the vehicle dynamic condition and/or bring the vehicle into a target or predetermined operating range, a chord progressively leads to a full harmony. The closeness of the output to full harmony indicates to the listener the closeness of the vehicle being operated in the optimal way or within an operating range.

As an extension to modifying the closeness to harmony of the audio metaphor, the rhythm, and beat and other parameters of the audio metaphor can be adjusted. In some embodiments the rhythm may coincide with a first driving input, the beat may correspond to a second driver input, etc. Other parameters may correspond to other driver inputs. Audio parameters may correspond to vehicle dynamics or environmental conditions.

In some embodiments, a specific instrument may correspond to a specific control input. For example, a guitar may correspond to wheel slip, drums may correspond RPM for optimal gear shifting point, and bass guitar may correspond to tire life. All three of wheel slip, optimal gear shifting RPM and/or tire life may be continuously optimized. An audio metaphor can be generated to convey information about all three of wheel slip, gear shifting RPM, and/or tire life. This can be done by modulating and/or mixing the corresponding instrumental sounds in the metaphor. For example, the driver may select a song they are familiar with. This song may play as the driver operates the racing vehicle. The audio metaphor may alter the playback to send information to the driver. If wheel slip is non-optimal, the guitar may sound either to low or too high in the song. Similarly, if the RPM is not optimal for gear shifting then the drums make be too high or too low. Similarly, if the tire life is not being optimized the bass guitar may be too high or too low. When all the inputs are optimal, the song may be played with non-modified sounds. In some embodiments, the music may be music that is computer generated on demand. In the case of newly generated music, the generation system may be consistent depending on the setting selected, such that once a driver becomes familiar with the system, they can be confident in their understandings of the vehicle dynamics as they relate to the metaphor.

In some embodiments, the music may be generated for a specific race. In some embodiments, the music may progress as the race progresses. The runtime of a generated song may be equal to the race length. The runtime of a generated song may be less than the race length. The runtime of a generated song may be greater than the race length. In some embodiments, the way an instrument is used may correspond to specific driver inputs. For example, a slide guitar sound may correspond to conveying information about steering inputs while fingerpicking guitar may correspond to braking inputs.

In some embodiments, the sound metaphor may have a standard base. This base could be, for example, a song, playlist, pink noise, silence, or any other base. In the optimal condition for all measured parameters the base may be played without modifications. Optimal condition may mean, within an operating range. Each measured parameter may be correlated with a specific sound or family of sounds. For example, non-optimal wheel slip may be associated with a specific whine or tone. As the wheel slip becomes further from optimal the whine may increase in intensity. The non-optimal wheel slip may be associated with two tones, one tone indicating above a threshold and one tone indicating below a threshold. All the measured and optimized vehicle performance parameters may be associated with different tones.

In some embodiments, the wheel slip can correspond to a sound or audio metaphor that can vary based on the degree of understeer or oversteer of the driver in operating the vehicle, based on for example, a template as discussed herein. For example, an instrument such as a violin or guitar associated with wheel slip can be sounded playing harmonious tones when there is relatively no understeer or oversteer. As the degree of understeer or oversteer increases, the tone can become correspondingly more disharmonious. In some cases, understeer can be associated with relatively low disharmonious chord sound metaphors. Oversteer can be associated with relatively high disharmonious chord sound metaphors. In some cases, depending on the degree of understeer or oversteer, octaves of the chords may be changed to further emphasize the degree to which performance is to be corrected.

The various tones may be overlapped when multiple parameters are out of the optimum and/or desired. In some cases, the messages conveyed simultaneously may be limited in number to 1, 2, 3, 4, 5, or 6 different messages being conveyed at once. In some embodiments, the messages may be prioritized, as discussed elsewhere in this disclosure. In some embodiments, the various vehicle performance parameters may be mapped to different instruments such that instead of merely a tone or a whine, the non-optimal conditions are signaled by a simulated or recorded instrument. In some embodiments, the various vehicle performance parameters may be mapped to different sounds such as laughter, crying, cries of pain, clapping, or other sounds. The various vehicle performance parameters may be matched to any sounds. The added sounds could also be other soundtracks, such as different songs. A user input device and data transfer devices may enable users to select their own tones, and soundtracks to correlate with various vehicle parameters. Using the user interface 110 the user may enter user preferences related which sounds the system should use, which metaphor construction techniques the system should use, and these preferences may be varied for each of the various potential messages to be conveyed.

In some embodiments, the sound metaphor may have a standard base. The standard base could be a song, a playlist, or any other soundtrack. This base may have various channels whose parameters can be modified independently of the other channels. Each of the vehicle performance parameters being conveyed to the driver can be correlated to a channel in the sound metaphor. When the driver is operating the car optimally, all the channels may be played in their original states. However, as the driver deviates from the optimal in one or more parameters the various channels of the sound may be modified. The modification could affect any parameter of the channel. For example, in the non-optimal state for one parameter, one channel may have increased or decreased volume, or higher or lower sound frequency. For example, if the vocals of a song may be tied to the wheel slip ratio, as the wheel slip ratio deviates from optimal the vocals may become higher volume or could become lower volume.

In some embodiments, the sound metaphor may be a specific assembly of notes. The notes may follow a predictable pattern. For example, the notes may repeatedly increase in frequency at a set time interval until reaching a maximum or desired threshold and resetting to a base low note and then rising again. Another example pattern may be a metronome. Another example pattern may be a metronome with a specific tone. Another example pattern may be a refrain or a chorus of a song. The sound metaphor may alter the base pattern to convey information to the driver as described herein. The driver who detects a change in the base pattern may be able to adjust the driver inputs to bring the pattern back to the base. The base pattern may be associated with a desired or optimal operating range. The base pattern may be modified by a system similar to the audio modifier 302 to generate the audio message 304. The audio message 304 may be the audio metaphor. The base audio 312 may be the base pattern described herein. The specific assembly of notes may enable the driver to predict what notes to expect. A repeating pattern may allow the driver to quickly interpret the audio metaphor to identify driver actions to take to return the audio metaphor, and the operated vehicle, to a desired or optimal operating condition or range.

In some embodiments, a single channel may be affected by multiple vehicle operation parameters. For example, non-optimal wheel slip may be communicated by altering the volume of a channel and the optimal gearshift RPM may be indicated by the playback speed of the channel. In some embodiments, a single parameter of a channel could be affected by more than one vehicle parameter. In some embodiments, which include a single channel parameter being affected by more than one vehicle parameter, there may be an additional sound that is played to indicate which vehicle parameter is affecting the channel parameter. For example, if both wheel slip and gearshift RPM affect volume of an audio channel, there may also be a whine that indicates it is wheel slip information being conveyed, and/or a thud that indicated gearshift RPM information is being conveyed.

The vehicle information monitored by the controller 104 can include the engine revolutions per minute (RPM). To utilize all available engine power for maximum acceleration, a driver can manually shift gears at the latest point. If the driver selects a gear too late, the rev limit engages, which limits the RPMs of the engine. With the rev limit engaged, acceleration is reduced. If the driver selects a gear too early, acceleration is also compromised by less than optimum and/or desired use of available engine torque. An audio message about the optimum and/or desired gearshift points can replace or supplement the other cues a driver normally uses for gear shifting. For example, drivers may feel an audio cue with varying tone, beat, or other variations as discussed herein. Once the tone, beat, or other variations as discussed herein reaches a predetermined level, the drivers are notified of the optimum and/or desired gearshift point. In some embodiments, drivers will not receive an audio message until the optimum and/or desired gearshift point is present. Not receiving an audio message until the point in time of an optimum and/or desired gearshift can increase the ability to communicate other audio messages that may be more relevant during certain driving situations.

An audio message can communicate feedback to drivers on whether an optimum and/or desired gearshift point was executed. For example, the audio message may communicate a tone, beat, etc. cue or other variations as discussed herein with, for example, becoming disharmonious as a driver performs a gearshift farther away from an optimum and/or desired gearshift point.

Optimum and/or desired gearshift points can also be utilized during deceleration. Maximizing the engine RPMs while downshifting gears in braking situations can help decelerate a car. The selected gear should match the wheel speed of the car with the optimum and/or desired engine RPM to help avoid compression lock up and engine damage through excessive engine RPM that can be generated by an imposed high wheel speed. An audio message can communicate to the driver an optimum and/or desired relationship between the wheel speed, engine RPM, and gear to be selected.

Based on an audio comparison of the two or more audio cues with varying tone, beat relating to the RPMs and the wheel speed(s), drivers can optimize deceleration by matching wheel speeds with optimum and/or desired RPM through a selection of an audio indicated optimum and/or desired gear. In some embodiments, drivers will not receive an audio message until the relative wheel speed(s) matches an optimum and/or desired engine RPM that would be achieved based on a next gearshift down (e.g., from fourth to third gear). Thus, drivers can be notified when maximum deceleration can be achieved. Not receiving an audio message until the point in time of maximum deceleration or optimum and/or desired match of wheel speeds to RPM can increase the ability to communicate other audio messages that may be more relevant during certain driving situations.

The controller 104 can monitor actual RPM and rate of RPM change to provide an audio message that indicates rate of approach to a gear shift point as well as the gear shift point itself. The controller 104 can monitor wheel speed from all four wheels to identify wheel spin and provide an audio message that assists with power management (e.g., braking, deceleration, acceleration, etc.) during starts and exits from turns and/or corners.

The vehicle information monitored by the controller 104 can include wheel slip relative to the surface of travel. A car can have an optimum and/or desired wheel slip point during braking and/or deceleration such that the wheels rotating slower than the surface they are travelling over maximizes the deceleration of the car. Similarly, a car can have an optimum and/or desired wheel slip point during acceleration such that the wheels rotating faster than the surface they are traveling over maximize the acceleration of the car. Further, a car can have an optimum and/or desired wheel slip or grip point in cornering situations such that wheels slip slightly at the corner to maximize grip and cornering ability. An audio message can communicate to the driver an optimum and/or desired wheel slip or grip being achieved in real time and the driver can immediately adjust the car's controls accordingly. The audio message can incorporate spatial sound to represent the wheel quadrants and associated level of wheel slip. For example, drivers can receive a tone, beat, etc. cue or other variations as discussed herein relating to the wheel slip relative to the surface of travel. The wheel slip for each individual wheel can be communicated through an audio message. The tone, beat, etc. or other variations as discussed herein of the audio message can increase as a particular wheel slips more relative to the surface of travel. Based on an audio cue indicating the wheel slip, drivers can adjust control inputs to optimize the wheel slip for more grip of the tire during deceleration, acceleration, and/or cornering.

In some embodiments, drivers will not receive an audio message until the relative speed between the front and back wheels is such that wheel slip is optimum and/or desired or alternatively, not optimum and/or desired. An audio message can, for example, consist of a particular one or a particular combination notes, chords, tones, and/or other variations as discussed herein when optimum and/or desired wheel slip is present or is not present. Thus, drivers can be notified when maximum deceleration, acceleration, and/or cornering can be achieved or not achieved. Not receiving an audio message until the point in time of maximum deceleration, acceleration, and/or cornering (or lack thereof) can increase the ability to communicate other audio messages that may be more relevant during certain driving situations.

The vehicle information monitored by the controller 104 can include brake line pressure to further maximize deceleration through the achievement of optimum and/or desired wheel slip. Brake line pressure achieved through the effort applied to the brake pedal can be the primary means of decelerating a vehicle. An optimum and/or desired brake line pressure can be at a point right up to which a wheel lock up would be likely. Brake line pressure achieving the point right before wheel lock up can help optimize wheel slip during deceleration as described herein. An audio message can communicate to the driver the optimum and/or desired possible brake line pressure and real time feedback about how quickly it is achieved. Furthermore, the brake line pressure can waver under braking when, for instance, “heel to toe” braking can be needed to match engine RPM while downshifting gears as described herein. The changing brake line pressure can be communicated to the driver in real time with, for example, a tone, beat, etc. cue or other variations as discussed herein. The changing brake line pressure can replace or supplement the wheel slip audio messages for each individual wheel described herein.

The vehicle information monitored by the controller 104 can include vehicle position as discussed herein, including as part of vehicle awareness data 114 as discussed herein. The vehicle position can include a track lateral position and a track longitudinal position. The vehicle position can include the track lateral position and the track longitudinal position through the corner. One or more corner apex targets can be communicated and/or stored by the controller 104. In some embodiments, the controller 104 can determine and/or cause the communication of one or more corner apex targets to the driver using sound metaphors. A corner apex can be the point (e.g., a specified or predetermined track latitude and/or longitudinal position) at which the vehicle should be closest to the inside of the particular corner (e.g., closest point to an inside edge of the track for the vehicle taking a turn at the corner) that provides the best track performance. The corner apex can also be referred to as a clipping point of the corner.

In some embodiments, a pre-defined/predetermined template and an associated chord or chords can provide information to the driver. The driver can be provided with a tuned path. The tuned path can be defined based on a series of driver actions, a series of vehicle positions, other data from sensors. In some embodiments, audio (sound metaphor(s)) is played to the driver based on the driver's actions, vehicle positions, and/or other data from sensors. In some embodiments, audio (sound metaphor(s)) is played to the driver based on preferred driver's actions, vehicle positions, and/or other data from sensors. The audio played can be determined based on one or more discrepancies between a target driver's actions, vehicle positions, and/or other data from sensors and a measured driver's actions, vehicle positions, and/or other data from sensors. The audio can be discordant/disharmonious when the driver strays from the template. The audio can be harmonious when the driver stays within the template.

In some embodiments, the controller 104 can use a combination of track lateral position and longitudinal positions through the corner to determine and/or cause the communication of one or more corner apex targets. One or more clipping points for a corresponding one or more corners of the track can be instructed to the driver using sound metaphors. A pre-defined template and associated chords can provide a tuned path for the driver to follow. For example, when the driver strays or deviates from the tuned path or the template based on vehicle track lateral position and/or longitudinal position, the chord can become discordant as discussed above.

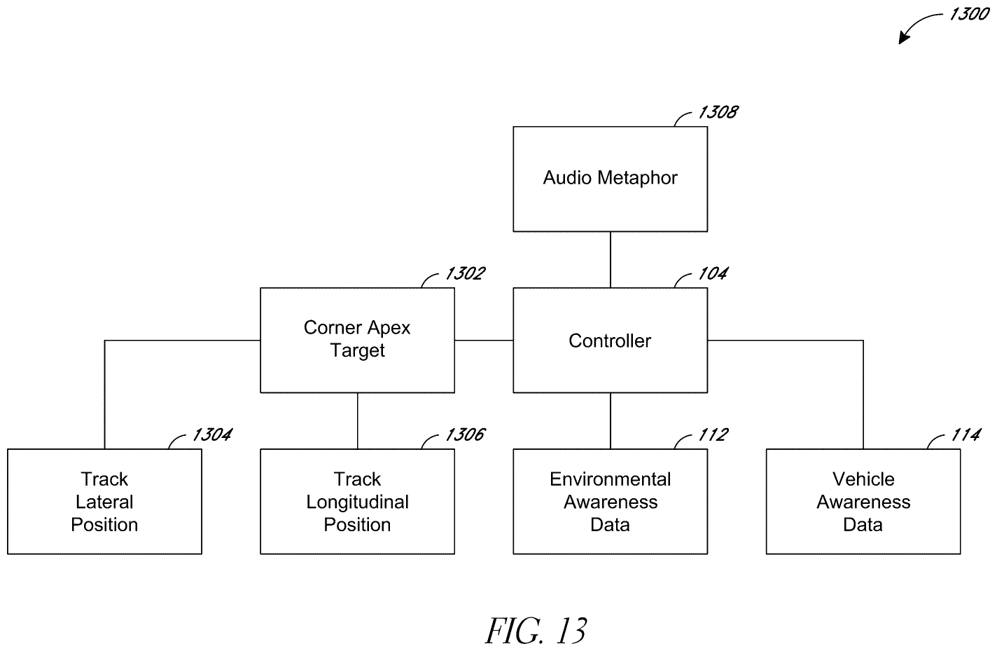

FIG. 13 illustrates an example embodiment for generating audio messages. FIG. 13 illustrates an example embodiment, system 1300 for communicating an audio metaphor corresponding to one or more corner apex targets. In some embodiments, the controller 104 receives or retrieves from memory a corner apex target 1302. The corner apex target 1302 can provide data corresponding to one or more desired vehicle conditions through the corner. For example, the corner apex target 1302 can correspond to a track lateral position 1304 of the vehicle. The corner apex target may also correspond to a track longitudinal position 1306 of the vehicle.

The controller 104 can receive or retrieve environmental awareness data 112 and/or vehicle awareness data 114, including track lateral position and longitudinal position of the vehicle, via for example GPS as discussed herein. In some embodiments, the controller 104 can determine an audio metaphor 1308 to be communicated based on at least one of one or more corner apex targets 1302, vehicle awareness data 114, and/or environmental awareness data 112. In some embodiments, the controller 104 can compare corner apex targets 1302 data to at least one of the vehicle awareness data 114 and/or the environmental awareness data 112. In some embodiments, the controller 104 can determine and cause communication of an audio metaphor 1308 based on at least a comparison between corner apex target 1302 data to at least one of the vehicle awareness data 114 and/or the environmental awareness data 112. The audio metaphor 1308 can convey to the driver a degree to which or amount that the comparison was outside or within a predetermined range or operating parameter for a particular corner apex target.

In some embodiments, one or more corner apex targets 1302 can be associated with desired corner entry. The controller 104 can determine or be provided program code for a corner entry audio metaphor based on a fastest result target or based on protecting a racing line. The fastest result target can be based on determining how to achieve a fastest time through a certain corner. Protecting the racing line can be based on determining how to maintain a desired path of the vehicle through the corner to achieve one or more racing line apex targets through the track and/or the corner. For example, the racing line can correspond to the optimal path around the track. The racing line apex target can correspond to entering a turn or corner at an outside edge of the corner of the track, touching the apex of the track on an inside edge of the corner of the track, and then exiting the turn by returning to the outside edge of the track. In some embodiments, the controller 104 can generate an audio and/or haptic message based on higher level logic as discussed herein. The controller 104 can determine when and which message metaphor is most useful for achieving the desired corner entry, including based on vehicle awareness data 114 and/or environmental awareness data 112. In some embodiments, the controller 104 can cause the system to communicate an audio and/or haptic message corresponding to the racing line apex target based on a predetermined position or location of the vehicle relative to a certain corner on the track. The corresponding position or location of the vehicle may be conveyed to the controller from vehicle sensors 106 and/or or from an external data source 115.

In some embodiments, the controller 104 can determine and correlate the racing line apex target and/or corner apex target to a predetermined target rate-of-turn at a corner entry for a certain corner of the track. The controller 104 can cause the system to communicate an audio metaphor corresponding to the predetermined target rate-of-turn. For example, the predetermined target rate-of-turn at corner entry can be communicated to the driver as a varying audible chord pitch. The chord can transition between flat and sharp notes during the driver's input, indicating whether the turn rate is too quick with sharp notes or is too slow with flat notes flat. The transition between sharp and flat notes can be continuous to communicate a continuous audio metaphor as discussed herein.

As discussed herein, the system can track the position or location of the vehicle on the track. In some embodiments, the controller 104 can cause the system to enable an audible (and/or haptic) replay of a previous operating performance relating to entry into a corner, a racing line apex target, and/or a corner apex target. For example, the system can replay a previous corner entry to a person by replaying the recorded audio metaphor. This can be useful for reminding the driver of their prior result and operation performance, particularly to give the driver information on the direction and magnitude of any correction needed while evaluating the driver's performance. In some embodiments, the replay may be communicated to the driver when the previous performance was not within a defined nominal set tolerance such as a predetermined threshold and/or range. In some embodiments, the system can replay a previous corner entry to a driver based, at least partially, on one or more of user preferences, environmental awareness data 112, and/or vehicle awareness data 114.

In some embodiment, the system can communicate any recorded (and stored in memory) series of audio or haptic messages and metaphors for replaying to the driver. This can used by the team when evaluating performance after the driving event as discussed herein. In some embodiments, the system can communicate a predefined audio and/or haptic message corresponding to a desired performance of the driver. For example, the system can communicate an audio metaphor corresponding to the desired driver inputs (e.g., steering, braking, throttling) of the vehicle as it enters a certain corner. This can be used in a simulator as discussed herein.

In some embodiments, the controller 104 can cause the system to play one or more musical guides or audio templates (or templates). A musical guide or template can be user defined. The musical guide or template can enable a driver to anticipate the desired performance, such as desired operation of the vehicle on the track. The controller 104 can vary the audio message played from the template based on a comparison of environmental awareness data 112 and/or vehicle awareness data 114 to the template. The template can include template data. The template data can correspond to a desired performance of the vehicle and/or the driver. The template can be associated with certain or predetermined portions or locations of the track (e.g., corners) based on, for example, longitudinal and/or latitudinal track positions and desired performance of the vehicle and/or driver based on location on the track. In some embodiments, the controller 104 can play audio that is off-key, out of tune, out of rhythm, or otherwise modified based on vehicle awareness data 114, environmental awareness data 112, and/or user preferences. For example, the controller 104 can play audio that is off key, out of rhythm, etc., when a driver fails to perform a desired (i.e., optimum) driver input to deliver the desired driver inputs associated with the template.

In some embodiments, the system can have one or more musical guides or templates (e.g., instructions stored in memory and executed by the processor) that can include associating particular instrumental sound (e.g. sound corresponding to a particular musical instrument or other sound) with specific data or driver inputs. A particular instrument can be assigned in the template for certain inputs. For example, a first driver input can be associate with a first instrument, and a second driver input can be associated with a second instrument. For example, a steering angle could be associated with violin audio. A brake input can be associated with a guitar audio or a base line. Various driver inputs can be associated with a selection of instruments. Associating instruments with particular data, or driver inputs, can be advantageous for allowing overlapping signals to be interpreted more easily, and by providing an intuitive predictive element with regard to what input the driver is to provide for desired performance and operation of the vehicle.

In some aspects, the controller 104 can cause the system to play pleasing or harmonious music when the driver is performing desired driver inputs and unpleasant or disharmonious sounds when not performing desired driver inputs for optimum performance or operation of the vehicle. The music played by the system can be based at least partially on user preferences, vehicle awareness data 114, and/or environmental awareness data 112 as discussed herein.

The system described herein can be advantageous because sounds or audio messages/metaphors can provide relatively more flexibility for conveying certain information effectively and can provide for overlapping of signals and messages. For example, both a slide guitar and a violin can be part of an audio message provided as part of a musical guide, corresponding to driver inputs of braking and steering as discussed herein, and both can be harmoniously sounding, disharmonious sounding, or one harmonious with the other disharmonious based on whether corresponding driver and steering inputs are within desired range of performance.

In some embodiments, the controller 104 can cause the system to use chords for audio metaphors relating to continuous time varying functions. For example, chords may be used as an audio metaphor or message when the driver's input is continuously assessed and translated to feedback that transitions across or between harmonious and discordant audio as discussed herein to provide, for example, feedback messages or post-event feedback messages. In some embodiments, the instrumental sounds used for various driver inputs, or other data, can suit the driver's preference. The sounds used to convey information to the driver can be selected for greater distinction over external noise. In some embodiments, the instrumental sounds of the audio messages used can be continuous sounds that are played or sounded by the system for a predetermined period of time or during continuous input of a certain driven input. For example, while the driver is pressing on the brake, a continuous sound (harmonious or disharmonious) can be played by the system. As another example, while the driver is turning the wheel a certain or predetermined amount or degree, a continuous sound (harmonious or disharmonious) can be played by the system. A continuous sound can be played based on locations on the track for one or more driver inputs as discussed herein. For example, a continuous sound associated with driver steering input can be continuously played based on the vehicle being proximate or at a corner of the track to provide a template for driver steering wheel control through the corner.

The controller 104 can modulate continuous sound over time and in response to driver inputs, user preferences, environmental awareness data 112, and/or vehicle awareness data 114 to provide, for example, feedback messages or post-event feedback messages. For example, certain audio metaphors or musical sounds can be emphasized by being louder depending on prioritization of desired messages discussed herein. In some embodiments, a certain driver input that is relatively most out of range or not being performed as desired compared to other driver inputs can be emphasized via modulation, including being played louder relative to other audio messages. In some embodiments, a sustained period of audio or musical sound can convey the desired audio metaphor for a particular driver input. The audio metaphor can be related to driver performance as discussed herein where the audible sustained period conveys the desired audio metaphor. In some embodiments, instrumental or musical sounds for continuous emulation of audio messages can include string instruments such as violin, viola, cello, guitar, slide guitar, bass, and/or banjo.

In some embodiments, the controller 104 can cause the system to use sound beats to mark points in time such as passing predetermined locations or points on the track, to represent an increasing or decreasing rate of driver input, and/or to provide other specific audio messages. In some embodiments, beats can be used in convey audio messages corresponding to target operating conditions or drive inputs and/or exceeding thresholds or ranges.). In some embodiments, instrumental or musical sounds for conveying beats can include percussion instruments such as timpani, xylophone, cymbals, triangle, drum, tambourine, maracas, gongs, chimes, celesta, and/or piano.

The attack and release of such audio events can convey the respective metaphor. Attack and release can refer to characteristics of sounds. For example, a drumbeat can have a fast attack sound (e.g., short/sharp start to the sound). A violin can have a slow attack sound (e.g., smooth). A guitar or piano can have a release sound (e.g., the sound continues for a time period after the string or key was struck).

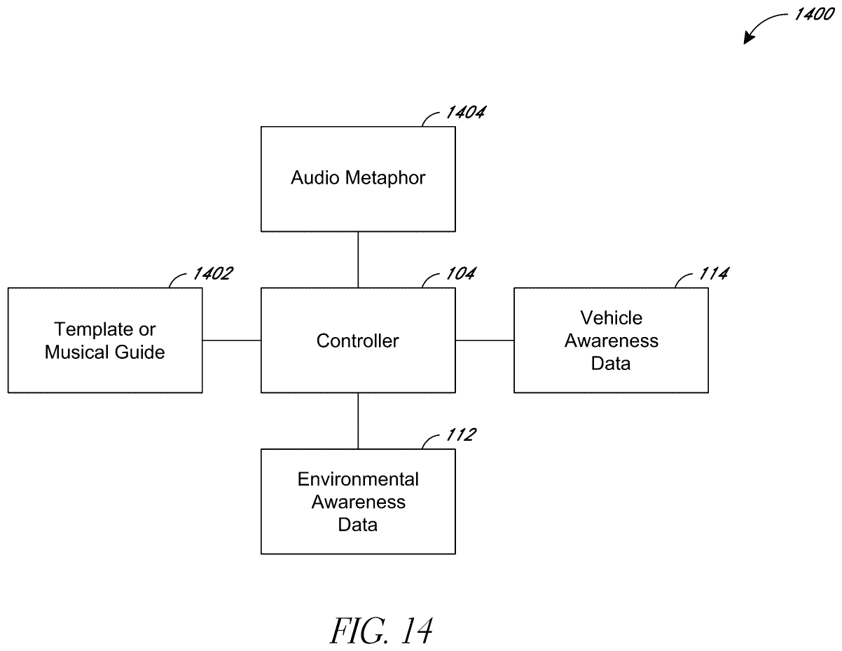

FIG. 14 illustrates an example embodiment for generating audio messages. FIG. 14 illustrates an example embodiment, system 1400 for communicating an audio metaphor based on a template or musical guide 1402. In some embodiments, the controller 104 receives or retrieves from memory one or more templates or musical guides 1402, vehicle awareness data 114 and/or environmental awareness data 112. The controller 104 can determine and cause the system to play an audio metaphor 1404 based on at least one of the templates 1402, vehicle awareness data 114, and/or environmental awareness data 112. In some embodiments, the controller 104 can compare template 1402 to at least one of the vehicle awareness data 114, or the environmental awareness data 112. In some embodiments, the controller 104 can determine and cause the system to play an audio metaphor 1404 based on at least a comparison between template 1402 to at least one of the vehicle awareness data 114 and/or the environmental awareness data 112 to convey desired performance information to the driver as discussed herein. The audio metaphor 1404 can convey to the driver a degree to which or amount that the comparison was outside or within a predetermined range or operating parameter/threshold associated with the template 1402.

For example, in some embodiments, an audio metaphor based on a template can be used for the dynamic application of vehicle brakes or brake pressure (e.g., brake line pressure). The controller 104 can determine and cause the system to convey an audio message (e.g., an audio metaphor) to the driver corresponding to a desired or predetermined pressure profile that generates a beneficial and characteristic transfer of vehicle weight (e.g., vehicle weight distribution on wheels of vehicle as discussed herein) during the braking event that enables peak deceleration and desired wheel sleep. The braking event can be associated with desired braking performance at certain portions or locations of the track, including corners, as discussed herein.

In some embodiments, the controller 104 can receive data indicative of a desired or predetermined brake pressure, pressure profile, or other data associated with the braking event. The controller 104 can provide an audio cue or message to the driver based on a comparison between a desired or predetermined desired brake pressure, pressure profile, or other data and a measured brake pressure, pressure profile, or other data.

The audio metaphor can be a continuous audio metaphor as discussed herein that guides the driver to maintain desired or predetermined weight distribution during the braking event to provide, for example, feedback messages or post-event feedback messages. For example, when weight distribution of the vehicle on the wheels is beyond (e.g., greater than) a predetermined threshold or range for a particular one or more wheels during the braking event, the controller 104 can cause the system to generate a disharmonious or discordant chord audio metaphor that can be continuous. When the weight distribution of the vehicle on the wheels is within (e.g., less than) the predetermined threshold or range for the particular one or more wheels (including all of the wheels to achieve desired weight distribution), the controller 104 can cause the system to generate a harmonious chord audio metaphor that can be continuous. The controller 104 can cause the system to continuously (e.g., linearly) vary and change the chord audio metaphor from harmonious to disharmonious depending on the amount or degree that the weight distribution of the vehicle on the wheels is beyond (e.g., greater than) the predetermined threshold or range for the particular one or more wheels during the braking event such that the chord changes from harmonious to relatively more disharmonious as the amount or degree increase, and vice versa, the chord changes from disharmonious to relatively more harmonious as the amount or degree decreases.

As another example, when weight distribution of the vehicle on the wheels is not within a predetermined profile for a particular one or more wheels during the braking event, the controller 104 can cause the system to generate a disharmonious or discordant chord audio metaphor that can be continuous. When the weight distribution of the vehicle on the wheels is within the predetermined profile for the particular one or more wheels (including all of the wheels to achieve desired weight distribution), the controller 104 can cause the system to generate a harmonious chord audio metaphor that can be continuous. The controller 104 can cause the system to continuously (e.g., linearly) vary and change the chord audio metaphor from harmonious to disharmonious depending on the amount or degree that the weight distribution of the vehicle on the wheels is not within the predetermined profile for the particular one or more wheels during the braking event such that the chord changes from harmonious to relatively more disharmonious as the amount or degree increase, and vice versa, the chord changes from disharmonious to relatively more harmonious as the amount or degree decreases.

The predetermined profile for weight distribution can include rate of change of weight distribution for the particular one or more wheels during the braking event. Depending on the desired or predetermined rate of change, the predetermine profile can include that the rate of change is greater than or less than a predetermined rate of change. Accordingly, the predetermined profile can include a predetermined range of change rate of weight distribution to provide dynamic feedback regarding the weight distribution rate of change. In some cases, the transition period or driver event associated with the change of weight distribution, and in particular rate of change, may be too short or occur too quickly for interactive driver feedback or real-time messaging as discussed herein. Accordingly, whether the driver action was performed to achieve the desired weight distribution may be a post-event feedback message that is reviewed by the driver and other team members after the driver-initiated event, including after the braking event, as well as after the driver has finished driving the vehicle and is reviewing the braking event of the racing or training session as discussed herein. While discussed here in the context of a braking event, the weight distribution feedback messages can be implemented by the system for other driver-initiated events discussed herein, including steering, accelerating, etc.

Based on an audio cue indicating brake line pressure, drivers can adjust control inputs to optimize the brake line pressure for more grip of the tire during deceleration and/or cornering. In some embodiments, drivers will not receive an audio message until the brake line pressure is optimum and/or desired or alternatively, not optimum and/or desired. When the audio messages are communicating optimum and/or desired brake line conditions, drivers can maintain the current optimum and/or desired brake line achieved. Not receiving an audio message until the point in time of optimum and/or desired brake line pressure (or lack thereof) can increase the ability to communicate other audio messages that may be more relevant during certain driving situations.

In some embodiments, the system can be aware of and communicate a braking marker that can be marked on approach. The braking marker can be a location the track for the driver to start braking. The controller 104 can cause the system to communicate the position to the driver with a series of audio messages such as a sequence of beats or beeps. For example, the controller 104 can cause the system to play a first audio message that is a relatively short low tone beat or beep. The controller 104 can cause the system to play a second audio message after the first audio message. The second audio message can be a relatively longer tone beat or beep while having the same low tone as the first audio message. The controller 104 can cause the system to play a third audio message after the second audio message. The third audio message can mark the brake point or location on the track. The third audio message can be a higher tone, while maintaining or increasing the relative tempo of the beats or beeps. The third audio message can be an audio cue that is distinctive and discrete from, for example, the first and second audio messages that communicated an approaching braking marker to provide the driver advance notice of the braking marker communicated with the third audio message.

The example first, second, and third audio message composed can overlap with other audible and haptic functions and messages. The braking marker audio communicated as beats or beeps can overlap, e.g. be played simultaneously with, a corner entry template chord as discussed herein. The corner entry template chord can use a continuous audio metaphor with pitch adjustment based on corner entry performance as discussed herein, while the beats or beeps for braking based on a braking marker can overlap with the continuous audio metaphor.

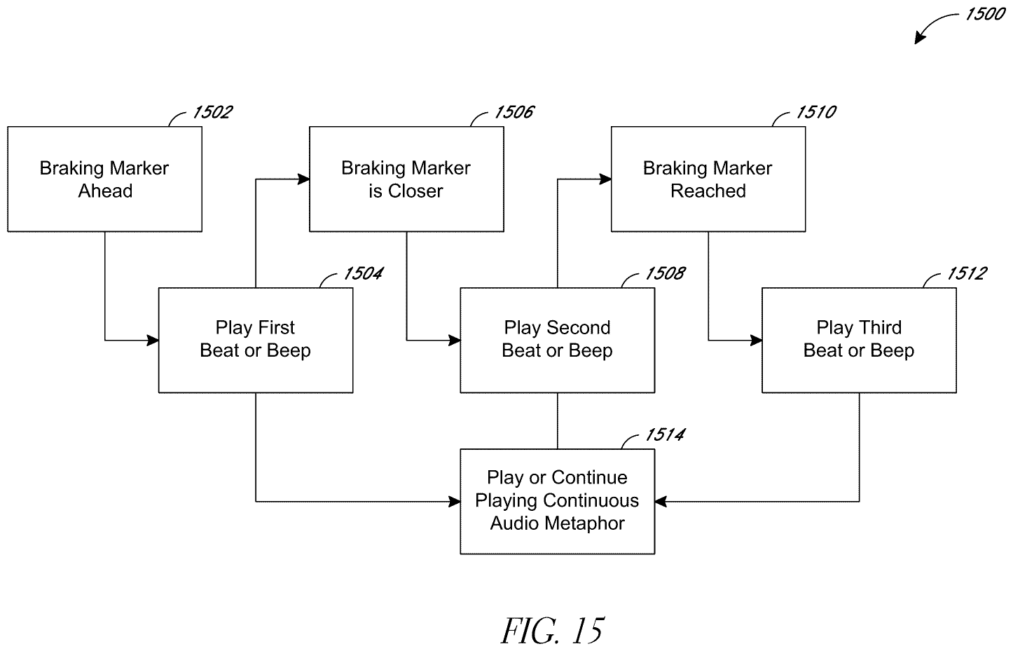

FIG. 15 illustrates an example embodiment of a process for generating audio messages. FIG. 15 illustrates an example embodiment, process 1500 for overlapping communication of audio beats or beeps with communication of a continuous audio message. At step 1502, the controller 104 can determine that a braking marker is ahead such as a first distance (e.g., on the track) away of the vehicle. The controller 104 can determine that the vehicle is the first distance away from the braking marker based on vehicle awareness data 114 and/or environmental awareness data 112. The controller 104 can play a first beep or a series of first beeps 1504 in response to the braking marker being the first distance away. The first beep or series of first beeps 1504 may be a first cue message having a tone or chord or a series of tones or chords. At step 1506, the controller 104 can determine that the braking marker is relatively closer such as at a second distance (e.g., on the track) away of the vehicle. The controller 104 can determine that the vehicle is the second distance away from the braking marker based on vehicle awareness data 114 and/or environmental awareness data 112. The controller 104 can play a second beep or a series of second beeps 1508 in response to the braking marker being the second distance away. The second beep or series of second beeps 1508 may be a second cue message having a tone or chord or a series of tones or chords. At step 1510, the controller 104 can determine that the braking marker is near or proximate (e.g., on the track) to the vehicle. The controller 104 can determine that the vehicle is at (including near or proximate) the braking marker based on vehicle awareness data 114 and/or environmental awareness data 112. The controller 104 can play a third beep or a series of third beeps 1512 in response to the vehicle being at the braking marker. The third beep or series of third beeps 1512 may be a third cue message having a tone or chord or a series of tones or chords. The controller 104 can play the third beep(s) 1512 to indicate that the driver should apply the brake driver input to operate the vehicle. In some embodiments, there may be one, two, three, or more distances from braking markers, and each distance can correspond to a different tone. In some embodiments, the first tone can be a short low tone; the second tone can be similar to the first tone except with a longer duration; and the third tone can be distinctive from the first and second tones.

While the system communicates the first, second, and/or third beep(s) 1504, 1508, and/or 1512, the controller 104 can cause the system to play or continue playing a continuous audio metaphor 1514 according to a template as discussed herein. The controller 104 can cause the system to play the continuous audio metaphor 1514 during any combination of the first, second, and/or third beeps or series of beeps as described in accordance with FIG. 15.

The vehicle information monitored by the controller 104 can include transitional input ramps in combination with or alternatively to continuous audio metaphors as discussed herein. The transitional input ramps can be associated with or correspond to sound beats as discussed herein. To maximize grip of the vehicle tires on the track, a driver can make relatively small transitional inputs into the steering, acceleration, and/or braking controls. The transitional inputs to the controls can result in “ramps” between zero and 100% of utilization of the available tire grip. The ramps resulting from the control inputs can mitigate jolts from the vehicle changing direction. An audio message can communicate to the driver the ramps between zero and 100% and augment any resulting jolts of the car. The amplification of jolts can aid the driver in correcting control inputs for a smoother transition from zero to 100% utilization of available tire grip under, for instance, braking and/or cornering. Better utilization of the available tire grip can result in better direction changes and reduced cornering times.

In some embodiments, drivers will not receive an audio message until the ramps are no longer smooth and/or results in jolts of the car. An audio message can, for example, consist of a particular note or a particular combination notes, pitches, or other variations as discussed herein when optimum and/or desired wheel grip is lost based on the ramp input, resulting in jolts of the car. Thus, drivers can be provided feedback on their ramp inputs to help condition optimum and/or desired use of the tire grip. Not receiving an audio message until the point in time of unsmooth ramps and/or resulting jolts of the car can increase the ability to communicate other audio messages that may be more relevant during certain driving situations.

The vehicle information monitored by the controller 104 can include tire wear. A driver should balance short term performance of the tire against long term benefits of prolonging tire life to match a race strategy. The race strategy can include a plan to minimize pit stops by prolonging tire life. Certain driving techniques can sacrifice immediate speed in order to prolong tire life, which can lead to time saved over an entire race when any time lost due to slower speeds is less than the time that would have been lost with extra pit stops to change tires. Thus, the optimum and/or desired wheel slip and grip points described herein can be balanced with tire life to achieve an overall better race result, not just an immediate improvement of acceleration, deceleration, and/or cornering. An audio message can communicate to the driver re-affirming cues based on certain driving techniques by, for instance, placing a predictive value on the remaining tire life. For example, drivers can receive a tone, beat, etc. cue or other variations as discussed herein relating to the remaining tire life. The tone, beat, or other variations can increase, decrease, or change harmony or other variations as discussed herein as the remaining tire life changes throughout racing or training. The tire life audio message can either be a constant audio cue, an audio cue generated in response to a driver or user input or communicated at certain time intervals or reference points during racing or training.

Based on an audio cue indicating the tire life, drivers can adjust control inputs to optimize the wheel slip for more grip of the tire during deceleration, acceleration, and/or cornering to prolong the tire life if such a race strategy is desired. Conversely, an audio message toward the end of racing or training indicating that more tire life remains than anticipated, the driver may drive more aggressively toward the latter part of the race or training session.

In some embodiments, drivers will not receive an audio message until the tire life is below a predetermined threshold, communicating to the driver an approximate run time before a pit stop is needed. Further, drivers can receive an audio message providing feedback when the driver maneuvers the car to decrease tire life beyond a predetermined threshold for a completed lap, a particular segment of the racetrack, and/or a particular maneuver such as at a particular corner. Not receiving an audio message until the point in time of deviation of the remaining tire life from a predetermined tire life can increase the ability to communicate other audio messages that may be more relevant during certain driving situations.

The vehicle information monitored by the controller 104 can include fuel and/or energy consumption, which along with tire life information, can be utilized to match a race strategy. A driver should balance short term car performance that possibly consumes more fuel and/or energy against long term benefits of decreasing or optimizing fuel and/or energy consumption to match a race strategy of, for example, minimizing pit stops. Even small changes in steering angle and throttle position can affect the fuel and/or consumption. Steering angle and throttle position can replace or supplement wheel slip/grip and/or transitional input ramps information described herein to determine immediate or long-term fuel and/or energy consumption. An audio message can communicate to a driver when inefficient fuel/energy consumption control inputs are detected and help reinforce more fuel and/or energy efficient control inputs. For example, drivers can receive a tone, beat, etc. cue or other variations as discussed herein relating to the remaining fuel and/or energy. The audio message can increase, decrease, or change in variations as discussed herein as the remaining fuel changes throughout racing on a per-lap basis, a track sector basis, or training scenarios.

The audio message can communicate to the driver the over-use or under-use of fuel and/or energy allocated per lap or interval. Fuel and/or energy allocated per lap may be based on remaining fuel/energy available. For vehicles using energy recovery systems, such as regenerative or kinetic systems, audio and/or haptic messages may be generated to convey information about driver and system performance related to energy management and deployment, such as regenerative energy harvesting and deployment. An audio message can communicate to the driver the energy recovery amount compared to a defined threshold and/or with respect to a distance travelled or track position, the capacity or status of recovery amount and the availability of deployment. The audio message may be situational such that the audio message is conveyed under certain conditions. The audio message may not be conveyed if fuel/energy is above a set threshold or is not of driver concern for the current driving requirements—possibly because of higher priority messages. The driver can initiate an audio message to advise the driver on current fuel/energy usage with respect remaining distance and performance requirements, to provide specific maneuver assessment, historic comparison, last lap or sector performance, or as a manual control to recall the last audio message. The remaining fuel and/or energy audio message can either be a constant audio cue or communicated at certain time intervals or reference points during racing or training.

An audio message can communicate feedback to the driver when the steering angle and/or throttle position is not optimum and/or desired, resulting in excessive fuel and/or energy consumption. Based on the audio cue indicating fuel and/or energy consumption in real time, such as an increasing tone, beat, or other variations as discussed herein level as fuel/energy consumption increases during a maneuver, drivers can adjust control inputs to optimize the steering angle, throttle position, wheel slip/grip, and/or transitional input ramps to decrease fuel and/or energy consumption if such a race strategy is desired. Conversely, an audio message toward the end of racing or training indicating that more fuel remains than anticipated, the driver may drive more aggressively toward the latter part of the race or training session.

In some embodiments, electric motorsport racing can use periods of energy harvesting and/or recovering, including throttle, coast, and boost recovery/usage. The availability and use of these periods can be adjusted dynamically to suit energy consumption requirements and race strategies. The controller 104 can cause the system to play audio metaphors that communicate these periods either via beats or continuous audio metaphors as discussed herein. For example, throttle and boost recovery audio messages can be continuous audio metaphors varying between harmonious and disharmonious sounds depending on degree or amount of deviation from desired or predetermined driver input (e.g., based on predetermined thresholds and/or ranges) as discussed herein. The coast audio message can be a beat or beep that changes based on anticipated driver input to, for example, apply the throttle at a certain point or location on the track (as discussed herein for braking markers). The audio metaphors can convey the status of boost availability and consumption rates at varying times, which can overlap other audible and haptic messages as discussed herein.

In some embodiments, drivers will not receive an audio message until the fuel and/or energy is below a predetermined threshold, communicating to the driver an approximate run time before a pit stop is needed. Further, drivers can receive an audio message providing feedback when the driver maneuvers the car to increase fuel and/or energy consumption beyond a predetermined threshold for a completed lap, a particular segment of the racetrack, and/or a particular maneuver such as at a particular corner. Not receiving an audio message until the point in time of deviation of the remaining fuel and/or energy from a predetermined fuel and/or energy life and/or permitted allocation (e.g., applications in electric motorsport can have restrictions on power below that which is available for use) can increase the ability to communicate other audio messages that may be more relevant during certain driving situations. In some embodiments, an audio system directed to minimizing fuel and/or energy consumption as described herein can be utilized in everyday commuter transportation or other professional driving fields, such as cargo transportation with large trucks.

The vehicle information monitored by the controller 104 can include reference points (e.g., corners as discussed herein) on a racetrack including for braking (deceleration), steering angle, acceleration, wheel slip/grip, tire life, and/or fuel and/or energy consumption, and vehicle location, which can be based on input from a global positioning system or otherwise derived (e.g., lap distance and/or in combination with inertia sensors). A driver can learn a racetrack and can look for visual markers on where, for instance, a latest or optimum and/or desired braking point is on the racetrack. An audio message can communicate the same visual cues more precisely as reference points based on vehicle location. An audio system can be configured based on an input of known GPS coordinates of optimum and/or desired reference points. In some embodiments, the audio system may support the ability for a driver to inform the system of particular locations to be used for generating certain types of cues. The system can be placed into a configuration mode in which the driver circles the track and marks specific locations (such as by pressing a button on the vest or steering wheel). The controller 104 can log the coordinates (GPS, derived position and/or field sensor/beacon based) to be used for generating reference point audio messages as described herein. The coordinates can be adjusted as needed if, for instance, the driver may have been mistimed entering an optimum and/or desired reference point. Further, the audio system can shift those reference points based on a desired race or training strategy as described herein such as prolonging or decreasing tire life and/or fuel and/or energy consumption. The shift in race strategy can occur in real time during racing or training such as when the audio system determines that more tire life and fuel and/or energy remains than needed at a particular reference point.

In some embodiments, an audio message can communicate progressive information to help the driver anticipate an approaching reference point such as a particular corner on the track. An audio message can also communicate to the driver an optimum and/or desired point to release the brake and/or release the accelerator pedal. The notes, chords, tones, and/or other variations as discussed herein can increase, decrease, or change as the reference point approaches or passes based on vehicle location. An audio message can communicate feedback to the driver whether the optimum and/or desired braking (deceleration), steering angle, acceleration, wheel slip/grip, tire life, and/or fuel and/or energy consumption at a particular reference point was achieved as discussed herein.

With reference point information on a racetrack for a steering angle, a driver can learn a racetrack and can look for visual markers on where, for instance, to take a particular steering angle into a corner. An audio message can communicate the same visual cues on when to take a steering angle more precisely as reference points based on vehicle location. An audio message can also communicate if the driver is under steering, over steering, steering too fast, steering too slow at a particular reference point as discussed herein. An audio message communicating reference points for a steering angle and/or over or under steering can help optimize cornering abilities and save time. For example, drivers can receive notes, chords, tones, and/or other variations as discussed herein relating to an approaching reference point and an optimum and/or desired steering angle. The notes, chords, tones, and/or other variations as discussed herein can increase, decrease, or change as the reference point approaches or passes based on vehicle location. Further, a different audio gradient cue can increase, decrease, or change in intensity as the driver turns the steering wheel in a particular direction communicating any deviation from an optimum and/or desired steering angle.

An audio message can communicate feedback to the driver whether the optimum and/or desired steering angle at a particular reference point, such as a corner as discussed herein, was achieved. For instance, notes, chords, tones, and/or other variations as discussed herein may relate how many degrees the driver missed the desired steering angle and/or relate to the time lost for less optimal steering control.

When controller 104 can monitor reference points on a racetrack for desired acceleration, a driver can learn a racetrack and can look for visual markers on where, for instance, an earliest or optimum and/or desired acceleration point is on the racetrack. An audio message can communicate the same visual cues for acceleration more precisely as reference points based on vehicle location. In some embodiments, an audio message can communicate progressive information to help the driver anticipate an approaching reference point for acceleration. An audio message can also communicate to the driver an optimum and/or desired point to stop accelerating. For example, notes, chords, tones, and/or other variations as discussed herein cue can increase, decrease, or change as the driver changes or maintains a certain acceleration at a particular reference point, communicating any deviation from optimum and/or desired acceleration.

An audio message can communicate feedback to the driver whether the optimum and/or desired acceleration at a particular reference point was achieved. For instance, notes, chords, tones, and/or other variations as discussed herein may relate to how much the actual speed of the car is different from an optimum and/or desired speed that could have been achieved based on an optimum and/or desired acceleration from a particular reference point. The audio message communicates to the driver whether acceleration was too high or too low from the particular reference point.

The controller 104 can monitor reference points on a racetrack to notify a driver of an approaching corner. An audio message can communicate reference points for approaching corners by surveying a course before a race event and configuring the audio system as described herein. Notifying the driver of an approaching corner can be useful for blind corners, long stage races such as a Road Rally race where a driver's memory is inaccurate, and/or nighttime racing where visibility can be poor. For example, drivers can receive notes, chords, tones, and/or other variations as discussed herein relating to an approaching corner. The notes, chords, tones, and/or other variations as discussed herein can increase, decrease, or as the car nears the approaching corner. Another audio cue can communicate the angle of the approaching corner. In some embodiments, the audio system can combine approaching corners information with reference point information for braking (deceleration), steering angle, acceleration, wheel slip/grip, tire life, fuel and/or energy consumption, and/or weight distribution to improve and optimize performance to achieve a corner apex target as discussed herein.

The controller 104 can monitor time gain or loss from reference laps and/or from reference points. Data available in a data logger and/or input available from a global positioning system or derived location can be utilized to log a reference lap or sector time. Sectors can be reference markers placed along a racetrack, splitting the racetrack into predetermined segments. An audio message can communicate to a driver time gain or loss from reference laps and/or from reference points in real time. The audio message can allow a driver to know in real time how a self-correction or a new technique affects a lap or sector time. Either with or without GPS input, the controller 104 can monitor distance traveled from a reference point and/or start line to determine the position of the car to, for example, compare a reference lap to a new lap. For example, drivers can receive notes, chords, tones, and/or other variations as discussed herein relating to a reference time at a particular reference point. A particular set of notes, chords, tones, and/or other variations as discussed herein can indicate by how many fractions of a second, whole seconds, and/or minutes that driver is ahead or behind on time at a reference point.

A racing vehicle can include sensors that can sense the relative position and change in position (trajectory) of a nearby racing vehicle on the track. Sensors can be located on the front, back, or sides of the racing vehicle. In some embodiments, the controller 104 can determine the position and change in position of a nearby racing vehicle(s) based on data received from GPS and/or beacons configured to track movements of the racing vehicles.

The controller 104 can receive the sensor signal data corresponding to the position and/or change in position of a nearby racing vehicle and to convey this message to the driver via one or more audio messages. When the nearby racing vehicle changes position relative to the driver's racing vehicle, the audio signal may change as the nearby racing vehicle approaches or as the nearby racing vehicle moves away.

For example, the driver may feel an audio cue with varying notes, chords, beats, or other variations as discussed herein relating to nearby racing vehicles in front of the driver's racing vehicle. Similar principles can be applied regarding nearby racing vehicles on the sides of the driver's racing vehicle. The controller 104 may additionally or alternatively detect that a nearby racing vehicle is attempting to pass and may immediately convey this information to the driver.

The controller 104 can alter performance-related audio messages as described herein based on the position of a nearby racing vehicle. For example, when the nearby racing vehicle is in front of the driver's racing vehicle, the controller 104 can alter the audio cue message regarding optimum and/or desired acceleration such as decreasing the rate of acceleration. As another example, the controller 104 can determine the optimum and/or desired steering angle and acceleration to maximize the possibility of passing the nearby racing vehicle. Similarly, the controller 104 can determine how to alter optimum and/or desired braking, timing of gear selection, entry and exit speeds, and/or entry and exit lines audio messages based on the position and change in position of the nearby racing vehicle. The altered audio messages can, for example, help avoid an accident if the unmodified queued audio message was communicating a trajectory that would have caused the driver to collide with the nearby car.

The controller 104 can alter the post-event feedback messages based on position and change in position of a nearby racing vehicle. For example, the controller 104 can generate a post-event feedback message indicating whether the driver took proper action to pass the nearby racing vehicle.

The vehicle information monitored by the controller 104 can include a weight distribution across four tires of the car. Acceleration can be optimized based on the weight distribution of a vehicle across the wheels of the vehicle. For example, on level surfaces of travel, acceleration can be optimized when the weight distribution of a vehicle is more equal across four wheels of the vehicle. On non-level surfaces of travel such as inclines or declines, a weight distribution may be optimal that is not equal across the four tires. An audio message can communicate to the driver an augmented sense of the weight distribution of the vehicle across the four wheels to replace or supplement a sense of weight distribution the driver may already have based on a roll of the vehicle. Audio message for weight distribution can also be related to the rate of change of the weight distribution. Communication of the weight distribution of the vehicle can help correct any driver tendency to not value the weight distribution in vehicle performance. For example, drivers can receive notes, beats, or other variations as discussed herein correlating to the weight distributed on a particular wheel. The notes, beats, or other variations as discussed herein can increase, decrease, or change as weight placed on each wheel changes. There can be four audio cues correlating to each wheel of the car that increase, decrease, or change as the weight placed on each wheel changes.

The weight distribution audio message can either be a constant or continuous audio cue, communicated at certain time intervals or reference points during racing or training, and/or communicated when the weight distribution on a particular wheel exceeds a predetermined amount or rate of change. Not receiving an audio message until the point in time of deviation of the weight distribution from a predetermined weight distribution can increase the ability to communicate other audio messages that may be more relevant during certain driving situations. In some embodiments, the audio message communicating the weight distribution as described herein can be reproduced on a training simulator to replace or supplement throwing the driver around to mimic steering into a corner.

In some embodiments, an audio metaphor can be applied based on unloading the steering input. For example, if after exiting a corner, the driver has not acted to restore the vehicle's balance or weight distribution to unload the steering within a predetermined time period or by a predetermine threshold, an audio metaphor emphasizing a changing or disharmonious sound (e.g. an increasingly loud painful sound) can be generated until the driver applies the correcting vehicle input to unload the steering wheel. In some embodiments, the changing disharmonious sound metaphor can be generated until a measured steering parameter matches a desired steering parameter. In some embodiments, the sound played to the driver can be selected from a variety of sounds including various sound effects or instruments as discussed herein.

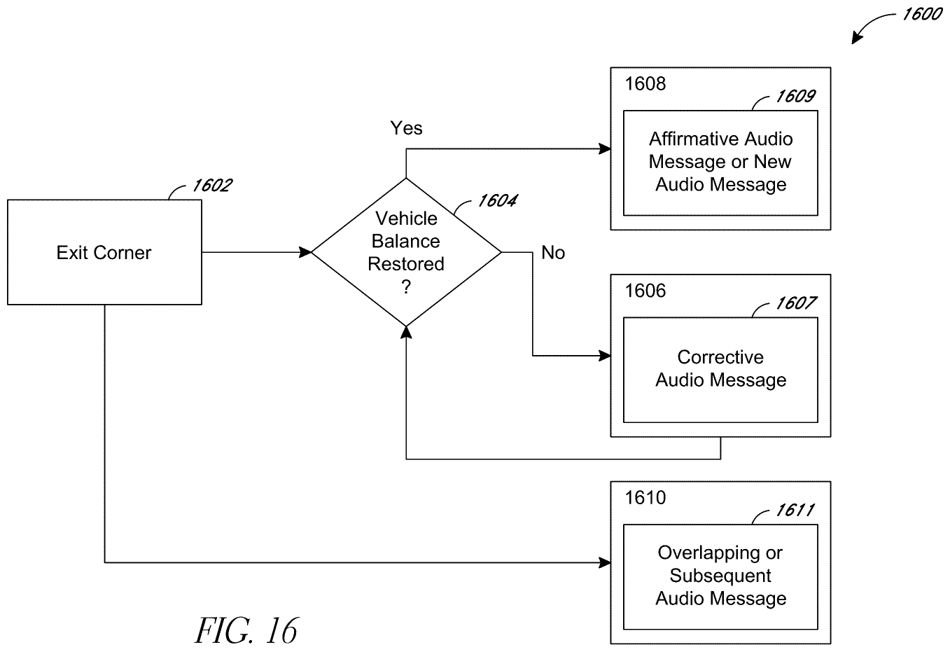

FIG. 16 illustrates an example embodiment of a process for generating audio messages. FIG. 16 illustrates an example embodiment, process 1600 for communicating an audio metaphor for restoring vehicle balance or weight distribution on the wheels of the vehicle as discussed herein. The controller 104 can determine that an exit corner event has occurred at step 1602. The controller 104 can determine when the vehicle balance has been restored or weight distribution at step 1604 within a predetermined time after corner exit or by a predetermined location on the track from the corner exit. If the vehicle balance has not been restored within the predetermined time after corner exit or by the predetermined location on the track from the corner exit, the controller 104 can cause the system to generate a corrective (e.g., disharmonious or unpleasant) audio message 1607 at step 1606.

In some embodiments, when the vehicle balance has been restored within the predetermined time after corner exit or by the predetermined location on the track from the corner exit, the controller 104 can cause the system to play an affirmative (e.g., harmonious or pleasant) audio message 1609 at step 1608. In some embodiments, the audio message 1609 can be a new audio metaphor for another driver input after the vehicle balance is restored. For example, after vehicle balance is restored at step 1604, the controller 104 can cause the system to play an audio message associated with predetermined driver input for applying the throttle out of a particular corner on the racetrack. In some embodiments, the controller 104 can cause the system to communicate vehicle balance and/or weight distribution of the vehicle into a corner to communicate desired driver inputs for optimum vehicle balance and/or weight distribution of the vehicle into the corner to achieve, for example, one or more corner apex targets as discussed herein. For example, the controller 104 can cause the system to communicate a corrective audio messages until a desired or predetermine vehicle balance and/or weight distribution is achieved when entering a corner.

As another example, when weight distribution of the vehicle on the wheels is not within a predetermined profile for a particular one or more wheels during the corner exiting, the controller 104 can cause the system to generate a disharmonious or discordant chord audio metaphor that can be continuous. When the weight distribution of the vehicle on the wheels is within the predetermined profile for the particular one or more wheels (including all of the wheels to achieve desired weight distribution), the controller 104 can cause the system to generate a harmonious chord audio metaphor that can be continuous. The controller 104 can cause the system to continuously (e.g., linearly) vary and change the chord audio metaphor from harmonious to disharmonious depending on the amount or degree that the weight distribution of the vehicle on the wheels is not within the predetermined profile for the particular one or more wheels during the corner exiting such that the chord changes from harmonious to relatively more disharmonious as the amount or degree increase, and vice versa, the chord changes from disharmonious to relatively more harmonious as the amount or degree decreases.

The predetermined profile for weight distribution can include rate of change of weight distribution for the particular one or more wheels during the corner exiting. Depending on the desired or predetermined rate of change, the predetermine profile can include that the rate of change is greater than or less than a predetermined rate of change. Accordingly, the predetermined profile can include a predetermined range of change rate of weight distribution to provide dynamic feedback regarding the weight distribution rate of change. In some cases, the transition period or driver event associated with the change of weight distribution, and in particular rate of change, may be too short or occur too quickly for interactive driver feedback or real-time messaging as discussed herein. Accordingly, whether the driver action was performed to achieve the desired weight distribution may be a post-event feedback message that is reviewed by the driver and other team members after the driver-initiated event, including after exiting the corner, as well as after the driver has finished driving the vehicle and is reviewing the corner exiting event of the racing or training session as discussed herein. While discussed here in the context of an exiting corner event, the weight distribution feedback messages can be implemented by the system for other driver-initiated events discussed herein, including steering, accelerating, etc.

In some embodiments, the controller 104 can determine vehicle balance or weight distribution using data or signals from one or more accelerometer sensors and/or gyro sensors (e.g., sensors 106). The controller 104 can determine pitch, yaw, and/or roll of the vehicle based on data or signals from the one or more sensors. The pitch, yaw, and roll can correspond to y-axis or lateral direction, z-axis or vertical direction, and x-axis or longitudinal direction, respectively, with respect to a coordinate system relative to the vehicle itself. The controller 104 can determine gravity forces on the vehicle based on data or signals from the one or more sensors. The controller 104 can compare pitch, yaw, roll, and/or gravity force values or levels (determined from the data or signals of the one or more sensors) to one or more corresponding predetermined thresholds or ranges. The controller 104 can cause the system to communicate one or more audio and/or haptic messages associated with vehicle balance and/or weight distribution as discussed herein when pitch, yaw, roll, and/or gravity force values or levels exceed or are beyond the corresponding predetermined thresholds or ranges for the driver to correct desired driver inputs, including during braking or corner exiting as discussed herein.

The audio messages 1607, 1609 can be continuous and change continuously from disharmonious to harmonious as discussed herein such as continuously varying from audio message 1607 to audio message 1609 as the driver corrects vehicle balance or weight distribution to provide, for example, feedback messages or post-event feedback messages. The controller 104 can continuously or periodically monitor and determine if vehicle balance has been restored at step 1604 until vehicle balance is restored. The audio messages 1607, 1609 can be user defined. The audio messages 1607, 1609 can change over time or based on vehicle location on the track as discussed herein. In some embodiments, the corrective audio message 1607 may be modulated based on when it is played, how many times it has been played, and/or based on vehicle awareness data 114 and/or environmental awareness data 112.

In some embodiments, the controller 104 can cause the system to play an overlapping audio message 1611 at step 1610. The audio message 1611 can be beat or beeps as discussed herein that overlaps with the audio messages 1607, 1609, which can be continuous. For example, the audio message 1611 can be a varying beat or beep message that indicates to the driver of an approaching marker on the track after exiting the corner 1602 such as discussed herein for a braking marker. The controller 104 can cause the system to play the audio message 1611 while or during playing audio message 1607 and/or audio message 1609 or can cause the system play the audio message after playing audio message 1607 and/or audio message 1609.

The controller 104 can utilize a control theory concept of observability to estimate data that cannot be sensed directly from sensors on a car because of regulations or cost of direct sensors. The control theory concept of observability can be a mathematical software model of the vehicle. The observer model of the vehicle can be used to estimate the optimal steering angle in a turn and/or a corner from GPS location data, wheel speeds, and/or accelerometer data with or without a gyro sensor. The sensors on a vehicle can include accelerometer sensors and/or gyro sensors. The observer model of the vehicle can predict impending wheel lock up during braking (before the wheels actually lock) to allow the driver an opportunity to modulate braking forces for optimal performance. The observer model of the vehicle can also incorporate environmental data such as track maps, rain sensors, and/or temperature sensors or probes to adjust the audio cues for the current race conditions.

The auxiliary information can include pit crew or other team member information. Pit crew information includes information that optimizes pit stop efficiency and improves pit stop safety with audio devices worn by pit crew members. An audio message can communicate to individual pit crew members the status of the other pit members' functions such as when will the vehicle's fuel tank be full in order to properly time changing of the tires to be ready at the same time. Correspondingly, an audio message can communicate to individual pit crew members the status of their own functions such as fuel feed, correct or incorrect wheel nut fitment, or tension on an air gun when tightening a wheel. The pit crew members can receive audio messages relating all pit function available or can toggle through the available audio messages with, for example, a menu correlated to the audio system. An audio message can also assist the car's controller to release the car safely and efficiently into a pit lane.

In some embodiments, pit control or pit crew can utilize audio beats as discussed herein to control the attention of the driver. For example, a low-pitched beat can instruct the driver to hold the brake while in the pit and denote a period where the pit crew and/or driver can review their instrumentation. A shift in beat tempo and pitch can be used to shift the driver's attention to the pit release crew member to allow for an efficient exit from the pit. In some embodiments, other audio message can be used for shifting the driver's attention as described herein.

In some embodiments, the user can tune how the audio metaphor will be generated. For example, the sensitivity to non-optimal/out of range conditions for various parameters may be adjustable. The system may be suitable for adjustment during use or between uses. I.e. adjustable during races, or between races. In some embodiments, the audio metaphor may be saved as it is generated. This may enable later playback. The data used to generate the sound metaphor may also be saved. If multiple people are receiving a sound metaphor based on a single vehicle the various users may select different vehicle optimization parameters to have integrated into their sound metaphor. In some embodiments, the rules used to generate the sound metaphor may be different for each user, this may include a different base being used, and/or other changes to the rules. In some embodiments, multiple user devices 135 may receive data related to a single or a set of vehicle(s), machine(s), or equipment control(s), and the user devices 135 may receive different data and may generate different audio metaphors depending on the type of user/operator (e.g., driver/operator, crew member, or simulator user) as well as other audio message controls discussed herein such as message prioritization.

The audio metaphors described herein may also change with respect to environmental factors. Information about environmental factors may be conveyed in the same way that information about vehicle performance factors are conveyed. Additionally, emotional metaphors, vehicle performance metaphors, and environmental metaphors may all be played simultaneously, may all use different channels, and may be prioritized between each other. Users may generate user specific rules to determine how prioritization is handled. Environmental factors may be communicated to the system either through the vehicle sensors 106, the CAN bus 105, or through the external data source 108.

In some embodiments, a training system may involve playing a recorded audio, haptic, and/or other metaphor.

In some embodiments, the sound metaphor may use directional, spatial, 3D surround or other audio effects to convey additional information. The messaging apparatus 100 may include multiple speakers 102 and/or incorporate surround sound technology to enhance sound metaphor content. The system may be configured to allow the user to perceive sounds as coming from different directions. This can enhance the sound metaphor. For example, the road ahead of the vehicle may have debris, surface contamination, animals, or unexpected water that may present a danger to the vehicle and occupants. The vehicle may have sensors, such as vehicle sensors 106 that detect the locations of the debris and/or other obstacles. When the sensors detect the obstacle, an audio metaphor may be generated. This audio metaphor may incorporate the use of directional, spatial, 3D surround or other sound to enable the driver to hear the direction of the debris. In effect, it may seem to the driver that the debris is emitting some sound. In some embodiments, the audio signal could include a simulated Doppler effect. Including a simulated Doppler effect may improve the driver's awareness of the obstacle.

The directional sound can be used to convey vehicle performance information to the operator. The metaphor can be used to express information related to road position, cornering position, vehicle dynamics information, and/or relative position to other objects in the vehicle's vicinity. A directional audio metaphor may indicate the type of danger, proximity, closure rate, optimal road position, and/or avoidance strategy. This directional awareness with or without Doppler effect could assist where vision is impaired due to weather conditions or the nature of the surface/terrain travelled.

Directional sound effects can be used to specify what another sound metaphor is conveying information about. For example, a whining noise may be associated with non-optimal wheel slip. Another example, a whining noise may be associated with loss of tire pressure. In both examples, the direction of the whine could indicate which wheel is affected. For example, if the front left wheel is non-optimally slipping, a whine may seem to emanate from the front left portion of the vehicle.

The directional sound effects can be applied in conjunction with the other sound metaphors creation techniques described elsewhere in this disclosure. For example, the controller 104 can cause the system to communicate haptic messages to communicate direction haptic messages that supplement or augment directional sound effects.

In some embodiments, the messaging apparatus 100 may be combined with the tactile apparatus as described in U.S. Pat. Nos. 8,552,847, 8,941,476, 9,327,703, and/or 9,734,678, each of which is incorporated in its entirety by reference and made a part of this specification. Once combined the two systems can extend the details of the feedback provided to the wearer. In some embodiments, the tactile apparatus and the audio apparatus may convey information about the same vehicle systems. In some embodiments, the tactile apparatus and the audio apparatus may convey information about different vehicle systems. For example, a moving vehicle may momentarily hit a retaining wall. The audio metaphor may announce the nature and severity of the impact, while the position of the haptics on the driver may be used as a reference to indicate where the impact was registered on the vehicle. Other combinations are also possible, wherein the audio indicates the direction, and the tactile indicates the severity. In some embodiments, the tactile apparatus acts similarly to another audio channel as described elsewhere in this disclosure.

In some embodiments, the audio metaphors can be associated with haptic messages. In some embodiments, the timing of audio message and haptic messages can be synchronized. In some embodiments, the controller 104 can cause the system to use an audio message to convey first data corresponding to, for example, a first driver input or first operating parameter and to use a haptic message may be used to convey second data corresponding to a second driver input or second operating parameter. The second data may be associated with the first data. In some embodiments the first (or second) data may be used to provide context for understanding the meaning of the second (or first) data. For example, a first data could indicate a magnitude, and the second data could represent what the magnitude corresponds to (e.g. steering input, throttle input, braking input, fuel level, speed, or other parameters).

The association of audio metaphors to haptic messages is advantageous to allow a greater variety of messages to be conveyed to the driver.

In some embodiments, the controller 104 can cause the system to convey wind direction and/or strength using haptic messages. The controller 104 can cause the system to play an audio message that corresponds to a whistling wind noise to provide the driver with the context that the haptic message corresponds to wind direction and/or strength. This application for combined audio and haptic messages can increase the capacity for the driver to use multiple different sensory feedback messages.

In some embodiments, the controller 104 can cause the system to use audio braking markers or braking messages as discussed herein with a combination of haptic messages. In some embodiments, the controller 104 can synchronize communication of the audio messages and the haptic messages. For example, the controller 104 can cause the system to play an audible beeping sound with a gradually increasing volume and cause the system to communicate a synchronized haptic message with a gradually increasing intensity. The controller 104 can cause the system to also synchronize the duration of the audible beeps with the duration of the haptic messages corresponding to the braking markers as discussed herein. For example, the controller 104 can cause the system to communicate the braking markers by using a combination of audible beep, beep, BEEP with a synchronized haptic tap, tap, TAP. The “BEEP” indicating a third beep with a tone distinctive from the first two “beeps.” In some embodiments, the distinctiveness of the third beep can be volume and/or tone. The “TAP” indicating a third tap with a haptic communication distinctive from the first two “taps.” In some embodiments, the distinctiveness of the third tap can be intensity and/or duration.

In some embodiments, the controller 104 can cause the system to play an audio message for communicating corner trajectory feedback corresponding to one or more corner apex targets as discussed herein that can be communicated with chord audio that transitions between harmonious and discordant. The time and/or rate of transition between harmony and discordance can depend on the drivers input in relation to the optimum trajectory template corresponding to the one or more corner apex targets. For example, the closer or more proximate the vehicle is to the corner apex target, the relatively faster or quicker the transition between a harmonious audio message and a discordant/disharmonious audio message. Simultaneously, while the trajectory is outside of the nominal target defined by the template for the one or more corner apex targets, the controller 104 can cause the system to communicate a haptic message to the driver to, for example, direct the driver toward a corner apex target. For example, the controller 104 can cause the system to communicate the haptic message as a beating or vibration on the respective left or right side of a haptic device such as a vest, with the left side vibration communicating to the driver to steer further or more left and the right side vibration communication to the driver to steer further or more the right. The haptic message (e.g., left or right beating) can occur in conjunction with the generation of discordant audio communicating to the driver other information about desired driver inputs to achieve the one or more corner apex targets.

In embodiments where the tactile apparatus is acting similarly to another audio channel, all the previous discussion about mixing and generating metaphors applies, except that the tactile apparatus outputs to the user are described in U.S. Pat. Nos. 8,552,847, 8,941,476, 9,327,703, and/or 9,734,678, each of which is incorporated in its entirety by reference and made a part of this specification.

In some embodiments, message generation by the controller 104 can include high level message generation. High level message generation can incorporate additional logic to, for example, prioritize messages and/or modify messages based on environmental and/or vehicle awareness data 112, 114 as discussed herein. The additional logic for high level message generation can control which messages are provided to the driver, in what order, and with what modification (e.g. modulation of volume, pitch, or another sound characteristic). High level message generation can incorporate additional logic to extend the benefit of the system. For example, incorporating additional logic to audio and/or haptic message generation and prioritization can be beneficial to situational awareness/functionality. Situational awareness/functionality can involve the collection of data based on environmental and/or vehicle awareness data 112, 114, including by various vehicle sensors 106 or from the external data source 115. Priority and importance of messages may be determined based at least partially on data from vehicle sensors 106 or an external data source 115 as discussed herein, for example, with reference FIG. 6. In some embodiments, the data can be indicative of a road condition, hazard, and/or other situations as discussed herein.

In some embodiments, the controller 104 can correlate and analyze multiple sources of data to dynamically adjust and/or change the message type and metaphor delivery. In some embodiments, generation of messages and metaphor delivery can be based, at least partially, on vehicle awareness data 114 and/or environmental awareness data 112. In some embodiments, priority and overlapping of messages can be based, at least partially, on vehicle awareness data 114 and/or environmental awareness data 112. For example, the controller 104 can vary audio and/or haptic messages corresponding to one or more braking markers depending on at least one of wind data, tire temperature, fuel load, and/or any other information, such as vehicle awareness data 114 and/or environmental awareness data 112.

In some embodiments, the recorded vehicle and environment data can be used for data analysis. This data can be used to produce audio metaphors on demand. The audio metaphor playback can be dynamically controlled. This may be particularly useful for training applications.

The audio metaphors can be comprised of synthesized content, music, and other sound files. The various audio sources may be mixed or used to vary one or more parameters of the audible content to produce the metaphors as described herein. The changes to parameters can include changes in volume, pitch, beat, vibrato, tone, pattern, repetition, scale, and directionality. Changes in parameters can also equate to changes in one channel of sound without changing others. In some cases, various instruments within the audio content may be subjected to changes, while other instruments are not. In some embodiments, one instrument's sound may be modified in a first way, and a second instrument's sound may be modified in a second way.

The systems and modes of creating audio metaphors can be applied to many application areas. For various application areas the sensors used, and the dynamics tracked may vary. In some embodiments, the system may be programmable to accept various inputs. In some embodiments, the system may be vehicle class specific. Some examples of vehicles that the system could be applied to include motorsport vehicles, road vehicles, simulators, construction vehicles, transport vehicles, watercraft, aircraft, submersible vehicles, space vehicles, autonomous vehicles, navigation and rally driving, and instrumental vehicle operation. For example, the audio messages described herein can be reproduced on a training simulator to replace or supplement throwing the driver around to mimic steering into a corner. Further, the driver, operator, athlete, and/or other person can use the audio system described herein when reviewing data in a simulator or on a screen to aid in improvement analysis. The engineer can use the audio system when reviewing data in a simulator or on a screen to aid in corrective engineering. Using the audio system while reviewing data in the data logger can aid the driver, operator, or athlete in remembering the vehicle's, machine's, and/or equipment's response based on the driver's, operator's, or athlete's, and/or other person's control inputs. Similarly, using the audio system can aid the engineer in understanding the vehicle's, machine's, and/or equipment's response based on the driver's, operator's, athlete's, or other person's control inputs and any vehicle, machine, and/or equipment mechanical and/or electronic modifications.

The system may also be applicable to remote controlled vehicles such as unmanned aerial vehicles or drones. In some embodiments the system may also be applicable to non-vehicle systems such as for monitoring a factory—wherein the sensors may, for example, detect line stoppages and the audio metaphor may convey this information to an operator or line worker.

The system may include various different audio delivery systems such as a digital radio system in the vehicle, a helmet audio system, an audio system configured to touch the user, a directional audio device, a device to transmit audio signal as an FM or AM signal for use by AM or FM receiver systems, a device to transmit audio signal over a network, a set of headphones, or a loudspeaker.

In some embodiments, one or more of these systems may be used. In some embodiments, these systems may be used simultaneously by the same or different users. The systems can be enabled, disabled, adjusted, or otherwise controlled such as adjusting audio metaphors as discussed herein by a user such as a non-driver, a non-operator, or a non-athlete, including engineers or other team members. In some embodiments, the audio signal may be transmitted over a network, such as the internet. The audio signal or data used to generate the audio signal can be transmitted over a network and can be accessible in real-time or on demand via computer or smart phone app. In some embodiments, the audio signal transmitted over a network may include a key for decoding the signal into sensor data or other data. The data, audio, haptic, and/or other message or metaphor may be transmitted to the driver, passenger, instructor, person using a simulator, engineer, team member, audience members, subscribers, app users, or other people.

### Variability of Embodiments

Various example embodiments have been disclosed in this disclosure. In many of these embodiments various components may be omitted. In many of these embodiments various components may be interchanged between the various embodiments. In many of these embodiments various components interconnections may be changed. All variations of the disclosed example embodiments, based on interchanging components between embodiments, removing various components of embodiments, or rearranging the components/steps of the embodiments are all to be understood as disclosed herein. Common technology, known in the art can be added to the disclosed embodiments. For example, throughout this disclosure, where a driver or a user is being referred to or discussed, this can include that a driver, user, passenger, instructor, person using a simulator, engineer, team member, pit crew, audience members, subscribers, app users, or other people receives the data, audio, haptic, and/or other message or metaphor with or without the driver or the user.

Depending on the embodiment, certain acts, events, or functions of any of the processes or algorithms described herein can be performed in a different sequence, can be added, merged, or left out altogether (e.g., not all described operations or events are necessary for the practice of the algorithm). Moreover, in certain embodiments, operations or events can be performed concurrently, e.g., through multi-threaded processing, interrupt processing, or multiple processors or processor cores or on other parallel architectures, rather than sequentially.

The various illustrative logical blocks, modules, routines, user interfaces, and algorithm steps described in connection with the embodiments disclosed herein can be implemented as electronic hardware, or combinations of electronic hardware and computer software. To illustrate this interchangeability, various illustrative components, blocks, modules, and steps have been described above generally in terms of their functionality. Whether such functionality is implemented as hardware, or as software that runs on hardware, depends upon the particular application and design constraints imposed on the overall system. The described functionality can be implemented in varying ways for each particular application, but such implementation decisions should not be interpreted as causing a departure from the scope of the disclosure.

Moreover, the various illustrative logical blocks, user interfaces, and modules described in connection with the embodiments disclosed herein can be implemented or performed by a machine, such as a general purpose processor device, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor device can be a microprocessor, but in the alternative, the processor device can be a controller, microcontroller, or state machine, combinations of the same, or the like. A processor device can include electrical circuitry configured to process computer-executable instructions. In another embodiment, a processor device includes an FPGA or other programmable device that performs logic operations without processing computer-executable instructions. A processor device can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor device may also include primarily analog components. For example, some or all of the algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, routine, or algorithm described in connection with the embodiments disclosed herein can be embodied directly in hardware, in a software module executed by a processor device (controller), or in a combination of the two, that command, control, or cause the system(s) and associated components described herein to perform one or more functions or features of the method, process, routine, or algorithm. A software module can reside in RAM memory, flash memory, ROM memory, EPROM memory, EEPROM memory, registers, hard disk, a removable disk, a CD-ROM, or any other form of a non-transitory computer-readable storage medium. An example storage medium can be coupled to the processor device such that the processor device can read information from, and write information to, the storage medium. In the alternative, the storage medium can be integral to the processor device. The processor device and the storage medium can reside in an ASIC. The ASIC can reside in a user terminal. In the alternative, the processor device and the storage medium can reside as discrete components in a user terminal.

### List of Example Numbered Embodiments

The following is a list of example numbered embodiments. The features recited in the below list of example embodiments can be combined with additional features disclosed herein. Furthermore, additional inventive combinations of features are disclosed herein, which are not specifically recited in the below list of example embodiments and which do not include the same features as the embodiments listed below. For sake of brevity, the below list of example embodiments does not identify every inventive aspect of this disclosure. The below list of example embodiments is not intended to identify key features or essential features of any subject matter described herein.

1. A system for improving racing vehicle driver performance, the system comprising:
a speaker capable of communicating audio signals to a driver of a racing vehicle; and
a controller that controls the speaker to communicate performance-related information to the driver during movement of the racing vehicle, said controller configured to receive vehicle information, including vehicle information collected by vehicle sensors of the racing vehicle, and configured to use the vehicle information to select audio messages to convey to the driver, the audio messages comprising (1) cue messages regarding actions to be performed by the driver, and (2) post-event feedback messages that convey to the driver whether actions were performed properly.

2. The system of embodiment 1, wherein the audio messages comprise variation in one or more of frequency, pitch, beat, volume, tone, rhythm pattern, chords, or sequence of chords.

3. The system of embodiment 1 or 2, wherein the vehicle information further comprises external data received by the racing vehicle.

4. The system of embodiment 3, wherein the controller is configured to adjust the audio messages in response to the external data.

5. The system of any one of Embodiments 1 to 4, wherein the controller is configured to make the audio messages harmonious in response to the racing vehicle being operated within a desired operating range.

6. The system of embodiment 5, wherein the controller is configured to include a cue message that varies from the harmonious audio messages in response to the racing vehicle being operated outside the desired operating range.

7. The system of embodiment 6, wherein an extent of the variation of the cue message corresponds to an extent of the racing vehicle being operated outside the desired operating range.

8. The system of embodiment 7, wherein the extent of the variation of the cue message corresponds to increasingly disharmonious or discordant chords being played with the extent of the racing vehicle being operated outside the operating range increasing.

9. The system of any one of embodiments 1 to 8, wherein the controller is configured to adaptively modify a selection of audio messages conveyed to the driver based on performance data collected by the controller.

The system of any one of embodiments 1 to 9, wherein the controller is configured to prioritize audio messages for audio delivery to the driver based at least partly on message type.

11. The system of embodiment 10, wherein the controller is configured to prioritize the audio messages in response to external data received by the racing vehicle, the external data comprising information corresponding to an environment of the racing vehicle.

12. The system of any one of embodiments 1 to 11, wherein the controller is configured to make the audio messages out of tune in response to the racing vehicle being operated outside a desired operating range.

13. The system of any one of embodiments 1 to 12, wherein the speaker comprises at least two speakers and wherein the audio messages comprise directional sound corresponding to at least one of racing vehicle awareness or environmental awareness of the racing vehicle.

14. The system of embodiment 13, wherein the racing vehicle awareness comprises at least one of a racetrack position of the racing vehicle or a cornering position of the racing vehicle.

15. The system of embodiment 13 or 14, wherein the audio messages corresponding to the racing vehicle awareness comprise at least one of a desired racetrack position of the racing vehicle or a location of impact on the racing vehicle.

16. The system of any one of embodiments 13 to 15, wherein the environmental awareness of the racing vehicle comprises at least one of position of an other racing vehicle relative to the racing vehicle or position of an object in an environment of the racing vehicle.

17. The system of embodiment 16, wherein the audio messages corresponding to the environmental awareness comprise a cue message to avoid the other racing vehicle or the object in the environment of the racing vehicle.

18. The system of embodiment 16 or 17, wherein the audio messages corresponding to the environmental awareness comprise a cue message corresponding to, relative to the racing vehicle, at least one of a proximity or approach rate of the other racing vehicle or the object in the environment of the racing vehicle.

19. The system of any one of embodiments 16 to 18, wherein the audio messages corresponding to the environmental awareness comprise a cue message to indicate a type of object in the environment of the racing vehicle.

The system of any one of embodiments 1 to 19, wherein a stereo system of the racing vehicle comprises the speaker.

21. The system of any one of embodiments 1 to 20, wherein a helmet configured to be worn by the driver of the racing vehicle comprises the speaker.

22. The system of any one of embodiments 1 to 21, wherein the actions performed by the driver comprise at least one of steering, gear shifting, braking, regenerative braking, energy harvesting, deployment of energy, or acceleration.

23. The system of any one of embodiments 1 to 22, wherein the controller is configured to communicate the audio messages corresponding to performance thresholds of the racing vehicle, the thresholds comprising at least one of wheel slip ratio, tire grip, tire wear, tire temperature, curb impact force, acceleration rate, deceleration rate, cornering rate, or fuel/energy consumption.

24. The system of any one of embodiments 1 to 23, wherein the audio messages correspond to at least one of environment of the racing vehicle, track position of the racing vehicle, or entry speed of the racing vehicle, or exit speed of the racing vehicle.

25. The system of any one of embodiments 1 to 24, wherein the controller is configured to adjust the audio messages depending on user inputs.

26. The system of any one of embodiments 1 to 25, wherein the cue messages regarding actions to be performed by the driver are continuously varied in response to actions performed by the driver or an environment of the racing vehicle.

27. The system of any one of embodiments 1 to 26, wherein the cue messages vary in at least one of rhythm or beat to prompt an action to be performed by the driver.

28. The system of any one of embodiments 1 to 27, wherein the controller is configured to associate tactile messages with the audio messages, the tactile messages corresponding to at least one of emphasis of the audio messages, classification of the audio messages, relating the audio messages to different post-event feedback messages.

29. The system of any one of embodiments 1 to 28, wherein the controller is configured to associate tactile messages with the audio messages, the tactile messages comprising directional cues corresponding to at least one of racing vehicle awareness or environmental awareness of the racing vehicle.

30. The system of embodiment 29, wherein the tactile messages corresponding to the racing vehicle awareness comprise at least one of a racetrack position of the racing vehicle or a cornering position of the racing vehicle.

31. The system of embodiment 29 or 30, wherein the tactile messages corresponding to the racing vehicle awareness comprise at least one of a desired racetrack position of the racing vehicle or a location of impact on the racing vehicle.

32. The system of any one of embodiments 29 to 31, wherein the tactile messages corresponding to the environmental awareness of the racing vehicle comprise at least one of position of an other racing vehicle relative to the racing vehicle or position of an object in an environment of the racing vehicle.

33. The system of embodiment 32, wherein the tactile messages corresponding to the environmental awareness comprise a cue message to avoid the other racing vehicle or the object in the environment of the racing vehicle.

34. The system of embodiment 32 or 33, wherein the tactile messages corresponding to the environmental awareness comprise a cue message corresponding to, relative to the racing vehicle, at least one of a proximity or approach rate of the other racing vehicle or the object in the environment of the racing vehicle.

35. The system of any one of embodiments 32 to 34, wherein the tactile messages corresponding to the environmental awareness comprise a cue message indicating a type of object in the environment of the racing vehicle.

36. The system of any one of embodiments 1 to 35, further comprising an other speaker capable of communicating audio signals to a person, wherein the controller is further configured to select which of the audio messages to convey to the person.

37. The system of embodiment 36, wherein the audio messages are communicated to the person substantially in real time as the driver controls the racing vehicle.

38. The system of embodiment 36 or 37, wherein the audio messages are communicated to the person after the driver controls the racing vehicle.

39. The system of any one of embodiments 1 to 38, further comprising a user interface for selecting a type of audio message conveyed to the driver.

40. A system for improving racing vehicle driver performance, the system comprising:
a speaker capable of communicating audio signals to a driver; and
a controller that controls the speaker to communicate performance-related information to the driver, the controller configured to receive vehicle information and configured to use the vehicle information to communicate to the driver an audio message that notifies the driver of a desired timing for performing a driver action corresponding to controlling a racing vehicle.

41. The system of embodiment 40, wherein the driver action is at least one of a braking action, an acceleration action, a gear shift action, or a steering action.

42. The system of embodiment 40 or 41, wherein the desired timing is based at least partly on a weight of the racing vehicle distributed on a particular wheel of the racing vehicle.

43. The system of any one of embodiments 40 to 42, wherein the desired timing is based at least partly on a desired tire slip relative to a surface of travel of a tire of the racing vehicle on a racetrack.

44. The system of embodiment 43, wherein the desired tire slip is based at least partly on spin of the tire relative to the surface of travel on the racetrack.

45. The system of any one of embodiments 40 to 44, wherein the audio message notifies the driver of a transitional input ramp associated with steering the racing vehicle to achieve the desired timing.

46. The system of any one of embodiments 40 to 45, wherein the controller is further configured to:
calculate the desired timing based at least partly on the vehicle information collected by vehicle sensors of the racing vehicle;
compare the desired timing to an actual time of the driver action; and
determine whether to output the audio message based on a result of the comparison.

47. The system of any one of embodiments 40 to 46, wherein the controller is further configured to:
calculate the desired timing based at least partly on the vehicle information collected by vehicle sensors of the racing vehicle;
compare the desired timing to an actual time of the driver action; and
select the audio message to convey to the driver based on a result of the comparison.

48. The system of any one of embodiments 40 to 47, wherein the controller is configured to determine the desired timing at least by comparing a vehicle location to a predetermined location on a racetrack.

49. The system of any one of embodiments 40 to 48, wherein the desired timing corresponds to a maximum of at least one of acceleration, deceleration, or cornering of the racing vehicle.

The system of embodiment 49, wherein the maximum of at least one of acceleration, deceleration, or cornering for the racing vehicle is based at least partly on a reference location on a racetrack.

51. The system of any one of embodiments 40 to 50, further comprising a user interface for selecting an audio message type conveyed to the driver.

52. The system of any one of embodiments 40 to 51, wherein the controller is configured to adaptively modify a selection of audio messages conveyed to the driver based on performance data collected by the controller.

53. The system of any one of embodiments 40 to 52, wherein the controller is configured to prioritize audio messages for delivery to the driver based at least partly on message type.

54. The system of any one of embodiments 40 to 53, further comprising an other speaker capable of communicating the audio message to a person, the audio message configured to notify the person of the desired timing for performing the driver action corresponding to controlling the racing vehicle.

55. The system of any one of embodiments 40 to 54, wherein the desired timing for performing the driver action is based at least partly on at least one of a position or a change in position of a nearby racing vehicle, and wherein the controller is further configured to receive information related to the nearby racing vehicle based at least partly on at least one of data received from vehicle sensors of the racing vehicle, the vehicle sensors configured to detect the position and the change in position of the nearby racing vehicle during movement of the racing vehicle, data received from a Global Positioning System (GPS) configured to track the position and the change in position of the nearby racing vehicle.

56. The system of any one of embodiments 40 to 55, wherein at least one of a vehicle stereo system, directional audio system, or helmet comprises the speaker.

57. The system of any one of embodiments 40 to 56, further comprising an audio device configured to have at least a portion physically contact the driver, the audio device comprising the speaker capable of communicating the audio signals to the driver

58. The system of any one of embodiments 40 to 57, wherein the controller is configured to determine whether the driver action was performed early or late relative to the desired timing by comparing a racing vehicle location at the time of the driver action to a pre-defined location on a racetrack.

59. The system of any one of embodiments 40 to 58, wherein the controller is configured to cause output of the audio signals in response to determining that the driver performed said action more than a threshold amount of time before the desired timing.

60. The system of any one of embodiments 40 to 59, wherein the controller is configured to cause output of the audio signals in response to determining that the driver performed said action more than a threshold amount of time after the desired timing.

61. A method for improving racing vehicle driver performance, the method comprising:
detecting a driver-initiated event performed during driving of a racing vehicle, said detecting performed by a computing device that monitors real time vehicle information associated with the racing vehicle;
determining a desired timing of the driver-initiated event based at least partly on collected sensor data;
comparing actual timing of the driver-initiated event to the desired timing; and
delivering to a driver an audio signal that is dependent upon a result of the comparison.

62. The method of embodiment 61, wherein the audio signal is applied to the driver substantially in real time.

63. The method of embodiment 61 or 62, wherein the audio signal is applied to the driver after the driver-initiated event.

64. The method of any one of embodiments 61 to 63, wherein the audio signal corresponds a degree to which the driver-initiated event was performed too early or too late relative to the desired timing.

65. The method of embodiment 64, wherein the desired timing is based at least partly on a pre-specified reference location on a racetrack.

66. The method of any one of embodiments 61 to 65, wherein delivering the audio signal comprises conveying audio messages that comprise variation in one or more of frequency pitch, beat, volume, tone, rhythm pattern, chords, or sequence of chords.

67. The method of any one of embodiments 61 to 66, wherein the vehicle information further comprises external data received by the racing vehicle.

68. The method of embodiment 67, wherein the audio signal is adjusted in response to the external data.

69. The method of any one of embodiments 61 to 68, wherein the audio signal is made harmonious in response to the racing vehicle being operated within a desired operating range.

70. The method of embodiment 69, wherein the audio signal comprises a feedback message that varies from the harmonious audio signal in response to the racing vehicle being operated outside the desired operating range.

71. The method of embodiment 70, wherein an extent of the variation of the feedback message corresponds to an extent of the racing vehicle being operated outside the desired operating range.

72. The method of embodiment 71, wherein the extent of the variation of the feedback corresponds to the extent of the racing vehicle being operated outside the desired operating range.

73. The method of any one of embodiments 61 to 72, wherein a selection of audio messages conveyed to the driver is adaptively modified based on performance data collected by the controller.

74. The method of any one of embodiments 61 to 73, wherein the audio messages are prioritized for audio delivery to the driver based at least partly on message type.

75. The method of embodiment 74, wherein the controller is configured to prioritize the audio messages in response to external data received by the racing vehicle, the external data comprising information corresponding to an environment of the racing vehicle.

76. The method of any one of embodiments 61 to 75, wherein the audio signal is made out of tune in response to the racing vehicle being operated outside a desired operating range.

77. The method of any one of Embodiments 61 to 76, wherein the audio signal is delivered to the driver via a speaker.

78. The method of embodiment 77, wherein the speaker comprises at least two speakers and wherein the audio signal comprises directional sound corresponding to at least one of racing vehicle awareness or environmental awareness of the racing vehicle.

79. The method of embodiment 78, wherein the audio signal corresponding to the racing vehicle awareness comprises at least one of a racetrack position of the racing vehicle or a cornering position of the racing vehicle.

80. The method of embodiment 78 or 79, wherein the audio signal corresponding to the racing vehicle awareness comprises at least one of a desired racetrack position of the racing vehicle or location of an impact on the racing vehicle.

81. The method of any one of embodiments 78 to 80, wherein the audio signal corresponding to the environmental awareness of the racing vehicle comprises at least one of position of an other racing vehicle relative to the racing vehicle or position of an object in an environment of the racing vehicle.

82. The method of embodiment 81, wherein the audio signal corresponding to the environmental awareness comprises a cue message to avoid the other racing vehicle or the object in the environment of the racing vehicle.

83. The method of embodiment 81 or 82, wherein the audio signal corresponding to the environmental awareness comprises a cue message corresponding to, relative to the racing vehicle, at least one of a proximity or approach rate of the other racing vehicle or the object in the environment of the racing vehicle.

84. The method of any one of embodiments 81 to 83, wherein the audio signal corresponding to the environmental awareness comprises a cue message indicating a type of object in the environment of the racing vehicle.

85. The method of any one of embodiments 61 to 84, wherein the driver-initiated event comprises at least one of steering, gear shifting, braking, or acceleration.

86. The method of any one of embodiments 61 to 85, wherein the audio signal corresponds to performance thresholds of the racing vehicle, the thresholds comprising at least one of wheel slip ratio, tire grip, tire wear, tire temperature, curb impact force, acceleration rate, deceleration rate, cornering rate, or fuel/energy consumption.

87. The method of any one of embodiments 61 to 86, wherein the audio signal corresponds to at least one of environment of the racing vehicle, track position of the racing vehicle, entry speed of the racing vehicle, or exit speed of the racing vehicle.

88. The method of any one of embodiments 61 to 87, wherein the audio signal is adjusted depending on user inputs.

89. The method of any one of embodiments 61 to 88, wherein cue messages regarding actions to be performed by the driver are continuously varied in response to actions performed by the driver or an environment of the racing vehicle.

90. The method of any one of embodiments 61 to 89, wherein cue messages vary in at least one of rhythm or beat to prompt an action to be performed by the driver.

91. The method of any one of embodiments 61 to 90, wherein tactile messages are associated with the audio signal, the tactile messages corresponding to at least one of emphasis of the audio signal, classification of the audio signal, or relating the audio signal to different post-event feedback messages.

92. The method of any one of embodiments 61 to 91, wherein tactile messages are associated with the audio signal, the tactile messages comprising directional sound corresponding to at least one of racing vehicle awareness or environmental awareness of the racing vehicle.

93. The method of embodiment 92, wherein the racing vehicle awareness comprises at least one of a racetrack position of the racing vehicle or a cornering position of the racing vehicle.

94. The method of any one of embodiments 61 to 93, wherein the audio signal has an audio pattern representing a late driver-initiated event and is applied in response to a determination that the driver-initiated event occurred more than a threshold amount of time after the desired timing.

95. The method of any one of embodiments 61 to 94, wherein the audio signal has an audio pattern representing an early driver-initiated event and is applied in response to a determination that the driver-initiated event occurred more than a threshold amount of time before the desired timing.

96. The method of any one of embodiments 61 to 95, wherein said desired timing is determined by the computing device based at least partly on vehicle location data.

97. A system for improving control of a vehicle, the system comprising:
a speaker capable of communicating audio signals to a person; and
a controller that controls the speaker to communicate performance-related information to the person, the controller configured to receive vehicle information and configured to use the vehicle information to communicate to the person an audio message that notifies the person of a desired timing for performing a driver action corresponding to controlling the vehicle.

98. The system of embodiment 97, wherein the driver action is at least one of a braking action, an acceleration action, a gear shift action, or a steering action.

99. The system of embodiment 97 or 98, wherein the desired timing is based at least partly on a weight of the vehicle distributed on a particular wheel of the vehicle.

100. The system of any one of embodiments 97 to 99, wherein the desired timing is based at least partly on a desired tire slip relative to a surface of travel of a tire of the vehicle.

101. The system of embodiment 100, wherein the desired tire slip is based at least partly on spin of the tire relative to the surface of travel.

102. The system of any one of embodiments 97 to 101, wherein the audio message notifies the person of a transitional input ramp associated with steering the vehicle to achieve the desired timing.

103. The system of any one of embodiments 97 to 102, wherein the controller is further configured to:
calculate the desired timing based at least partly on the vehicle information collected by vehicle sensors;
compare the desired timing to an actual time of the driver action; and
determine whether to output the audio message based on a result of the comparison.

104. The system of any one of embodiments 97 to 103, wherein the controller is further configured to:
calculate the desired timing based at least partly on the vehicle information collected by vehicle sensors;
compare the desired timing to an actual time of the driver action; and
select the audio message to convey to the person based on a result of the comparison.

105. The system of any one of embodiments 97 to 104, wherein the controller is configured to determine the desired timing at least by comparing a vehicle location to a predetermined location.

106. The system of any one of embodiments 97 to 105, wherein the desired timing corresponds to a maximum of at least one of acceleration, deceleration, or cornering of the vehicle.

107. The system of embodiment 106, wherein the maximum of at least one of acceleration, deceleration, or cornering for the vehicle is based at least partly on a reference location.

108. The system of any one of embodiments 97 to 107, further comprising a user interface for selecting a type of audio message conveyed to the person.

109. The system of any one of embodiments 97 to 108, wherein the controller is configured to adaptively modify a selection of audio messages conveyed to the person based on performance data collected by the controller.

110. The system of any one of embodiments 97 to 109, wherein the controller is configured to prioritize audio messages for tactile delivery to the person based at least partly on message type.

111. The system of any one of embodiments 97 to 110, wherein the desired timing for performing the driver action is based at least partly on at least one of a position or a change in position of a nearby vehicle, and wherein the controller is further configured to receive information related to the nearby vehicle based at least partly on data received from vehicle sensors of the vehicle, vehicle sensors configured to detect the position and the change in position of the nearby vehicle during movement of the vehicle, based at least partly on data received from a Global Positioning System (GPS) configured to track the position and the change in position of the nearby vehicle, or based at least partly on both data from the vehicle sensors and the data from the GPS.

112. The system of any one of embodiments 97 to 111, wherein at least one of a vehicle stereo system, directional audio system, or helmet comprises the speaker.

113. The system of any one of embodiments 97 to 112, further comprising an audio device configured to have at least a portion physically contact the person, the audio device comprising the speaker capable of communicating audio signals to the person.

114. The system of any one of embodiments 97 to 113, wherein the controller is configured to determine whether the driver action was performed early or late relative to the desired time by comparing a vehicle location at the time of the driver action to a pre-defined location.

115. The system of any one of embodiments 97 to 114, wherein the controller is configured to cause output of the audio signals in response to determining that a driver of the vehicle performed the driver action more than a threshold amount of time before the desired time.

116. The system of any one of embodiments 97 to 115, wherein the controller is configured to cause output of the audio signals in response to determining that a driver of the vehicle performed the driver action more than a threshold amount of time after the desired time.

117. The system of any one of embodiments 97 to 116, wherein the person is a driver of the vehicle.

118. The system of any one of embodiments 97 to 117, wherein the person is an individual that is not driving the vehicle.

119. The system of embodiment 118, wherein the individual is situated in a simulator associated with the vehicle.

120. The system of embodiment 118 or 119, wherein the individual is situated in the vehicle while a driver controls the vehicle.

121. The system of any one of embodiments 97 to 120, wherein the vehicle comprises at least one of a racing vehicle, motorsport vehicle, commuter or transport vehicle, electric vehicle, hybrid vehicle, construction vehicle, watercraft, aircraft, submersible vehicle, space vehicle, autonomous vehicle, or instructional vehicle.

122. The system of any one of embodiments 97 to 121, wherein the audio message corresponds to performance thresholds of the vehicle, the performance thresholds comprising at least one of wheel slip ratio, tire grip, tire wear, tire temperature, curb impact force, acceleration rate, deceleration rate, cornering rate, or fuel/energy consumption.

123. The system of any one of embodiments 97 to 122, wherein the audio message corresponds to at least one of environment of the vehicle, track position of the vehicle, or entry speed of the vehicle, or exit speed of the vehicle.

124. The system of any one of embodiments 97 to 123, wherein the controller is configured to adjust the audio message depending on user inputs.

125. The system of any one of embodiments 97 to 124, wherein the audio message regarding actions to be performed by a driver of the vehicle is continuously varied in response to actions performed by the driver or an environment of the vehicle.

126. The system of any one of embodiments 97 to 125, wherein the audio message varies in at least one of rhythm or beat to prompt an action to be performed by a driver of the vehicle.

127. The system of any one of embodiments 97 to 126, wherein the controller is configured to associate a tactile message with the audio message, the tactile message corresponding to at least one of emphasis of audio message classification of the audio message, relating the audio message to different post-event feedback message.

128. The system of any one of embodiments 97 to 127, wherein the controller is configured to associate a tactile message with the audio message, the tactile message comprising directional sound corresponding to at least one of vehicle awareness or environmental awareness of the vehicle.

129. The system of embodiment 128, wherein the tactile message corresponding to the vehicle awareness comprises at least one of a road position of the vehicle or a cornering position of the vehicle.

130. The system of embodiment 128 or 129, wherein the tactile message corresponding to the vehicle awareness comprises at least one of a desired position of the vehicle or location of impact on the vehicle.

131. The system of any one of embodiments 128 to 130, wherein the tactile message corresponding to the environmental awareness of the vehicle comprises at least one of position of an other vehicle relative to the vehicle or position of an object in an environment of the vehicle.

132. The system of embodiment 131, wherein the tactile message corresponding to the environmental awareness comprises a cue message to avoid the other vehicle or the object in the environment of the vehicle.

133. The system of embodiment 131 or 132, wherein the tactile message corresponding to the environmental awareness comprises a cue message corresponding to, relative to the vehicle, at least one of a proximity or approach rate of the other vehicle or the object in the environment of the vehicle.

134. The system of any one of embodiments 131 to 133, wherein the tactile message corresponding to the environmental awareness comprises a cue message to indicating a type of object in the environment of the vehicle.

135. The system of any one of embodiments 97 to 134, wherein the audio signals are communicated to the person substantially in real time as a driver of the vehicle controls the vehicle.

136. The system of any one of embodiments 97 to 135, wherein the audio signals are communicated to the person after a driver of the vehicle controls the vehicle.

137. A method for improving driver performance of a vehicle, the method comprising:
detecting a driver-initiated event performed during driving of a vehicle, said detecting performed by a computing device that monitors real time vehicle information associated with the vehicle;
determining a desired timing of the driver-initiated event based at least partly on collected sensor data;
comparing actual timing of the driver-initiated event to the desired timing; and
delivering to a person an audio signal that is dependent upon a result of the comparison.

138. The method of embodiment 137, wherein the audio signal is applied to the person substantially in real time.

139. The method of embodiment 137 or 138, wherein the audio signal is applied to the person after the driver-initiated event.

140. The method of any one of embodiments 137 to 139, wherein the audio signal corresponds a degree to which the driver-initiated event was performed too early or too late relative to the desired timing.

141. The method of embodiment 140, wherein the desired timing is based at least partly on a pre-specified reference location.

142. The method of any one of embodiments 137 to 141, wherein delivering the audio signal comprises conveying audio messages that comprise variation in one or more of frequency, pitch, beat, volume, tone, rhythm pattern, chords, or sequence of chords.

143. The method of any one of embodiments 137 to 142, wherein the vehicle information further comprises external data received by the vehicle.

144. The method of embodiment 143, wherein the audio signal is adjusted in response to the external data.

145. The method of any one of embodiments 137 to 144, wherein the audio signal is made harmonious in response to the vehicle being operated within a desired operating range.

146. The method of any one of embodiments 145 to 145, wherein the audio signal comprises a cue message that varies from the harmonious audio signal in response to the vehicle being operated outside the desired operating range.

147. The method of embodiment 146, wherein an extent of the variation of the cue message corresponds to an extent of the vehicle being operated outside the desired operating range.

148. The method of embodiment 147, wherein the extent of the variation of the cue message corresponds to the extent of the vehicle being operated outside the desired operating range.

149. The method of any one of embodiments 137 to 148, wherein a selection of audio messages conveyed to the person is adaptively modified based on performance data collected by the controller.

150. The method of any one of embodiments 137 to 149, wherein the audio signal is prioritized for audio delivery to the person based at least partly on message type.

151. The method of embodiment 150, wherein the controller is configured to prioritize the audio signal in response to external data received by the vehicle, the external data comprising information corresponding to an environment of the vehicle.

152. The method of any one of embodiments 137 to 151, wherein audio signal is made out of tune in response to the vehicle being operated outside a desired operating range.

153. The method of any one of embodiments 137 to 152, wherein the audio signal is delivered to the person via a speaker.

154. The method of embodiment 153, wherein the speaker comprises at least two speakers and wherein the audio signal comprises directional sound corresponding to at least one of vehicle awareness or environmental awareness of the vehicle.

155. The method of embodiment 154, wherein the vehicle awareness comprises at least one of a road position of the vehicle or a cornering position of the vehicle.

156. The method of embodiment 154 or 155, wherein the audio signal corresponding to the vehicle awareness comprises at least one of a desired position of the vehicle or location of impact on the vehicle.

157. The method of any one of embodiments 153 to 156, wherein the environmental awareness of the vehicle comprises at least one of position of an other vehicle relative to the vehicle or position of an object in an environment of the vehicle.

158. The method of embodiment 157, wherein the audio signal corresponding to the environmental awareness comprises a cue message to avoid the other vehicle or the object in the environment of the vehicle.

159. The method of embodiment 157 or 158, wherein the audio signal corresponding to the environmental awareness comprises a cue message corresponding to relative to the vehicle at least one of a proximity or approach rate of the other vehicle or the object in the environment of the vehicle.

160. The method of any one of embodiments 157 to 159, wherein the audio signal corresponding to the environmental awareness comprises a cue message to indicating a type of object in the environment of the vehicle.

161. The method of any one of embodiments 137 to 160, wherein the driver-initiated event comprises at least one of steering, gear shifting, braking, or acceleration.

162. The method of any one of embodiments 137 to 161, wherein the audio signal corresponds to performance thresholds of the vehicle, the performance thresholds comprising at least one of wheel slip ratio, tire grip, tire wear, tire temperature, curb impact force, acceleration rate, deceleration rate, cornering rate, or fuel/energy consumption.

163. The method of any one of embodiments 137 to 162, wherein the audio signal corresponds to at least one of environment of the vehicle, track position, or entry or exit speed.

164. The method of any one of embodiments 137 to 163, wherein the audio signal is adjusted depending on user inputs.

165. The method of any one of embodiments 137 to 164, wherein cue messages regarding actions to be performed by a driver of the vehicle are continuously varied in response to actions performed by the driver or an environment of the vehicle.

166. The method of any one of embodiments 137 to 165, wherein cue messages vary in at least one of rhythm or beat to prompt an action to be performed by a driver of the vehicle.

167. The method of any one of embodiments 137 to 166, wherein tactile messages are associated with the audio signal, the tactile messages corresponding to at least one of emphasis of the audio signal, classification of the audio signal, relating the audio signal to different post-event feedback messages.

168. The method of any one of embodiments 137 to 167, wherein tactile messages are associated with the audio signal, the tactile messages comprising directional sound corresponding to at least one of vehicle awareness or environmental awareness of the vehicle.

169. The method of Embodiment 168, wherein the vehicle awareness comprises at least one of a road position of the vehicle or a cornering position of the vehicle.

170. The method of any one of embodiments 137 to 169, wherein the audio signal has an audio pattern representing a late driver-initiated event and is applied in response to a determination that the driver-initiated event occurred more than a threshold amount of time after the desired timing.

171. The method of any one of embodiments 137 to 170, wherein the audio signal has an audio pattern representing an early driver-initiated event and is applied in response to a determination that the driver-initiated event occurred more than a threshold amount of time before the desired timing.

172. The method of any one of embodiments 137 to 171, wherein said desired timing is determined by the computing device based at least partly on vehicle location data.

173. The method of any one of embodiments 137 to 172, wherein the person is a driver of the vehicle.

174. The method of any one of embodiments 137 to 173, wherein the person is an individual that is not driving the vehicle.

175. The method of embodiment 174, wherein the individual is situated in a simulator associated with the vehicle.

176. The method of embodiment 174 or 175, wherein the individual is situated in the vehicle while a driver controls the vehicle.

177. The method of any one of embodiments 137 to 176, wherein the vehicle comprises at least one of a racing vehicle, motorsport vehicle, commuter or transport vehicle, electric vehicle, hybrid vehicle, construction vehicle, watercraft, aircraft, submersible vehicle, space vehicle, autonomous vehicle, or instructional vehicle.

178. A system for improving control of a vehicle, the system comprising:
a speaker capable of communicating audio signals to a person; and
a controller that controls the speaker to communicate performance-related information to the person, the controller configured with template information, the controller further configured to receive vehicle information and configured to generate an audio message based on the vehicle information and template information, the audio message communicating performance related information to a person.

179. The system of embodiment 178, wherein the performance related information is a desired timing for performing a driver action corresponding to controlling the vehicle.

180. The system of embodiment 178 or 179, wherein the vehicle information comprises vehicle awareness data.

181. The system of any one of embodiments 178 to 180, wherein the vehicle information comprises vehicle environmental data.

182. The system of any one of embodiments 178 to 181, wherein the controller is configured to compare template information to the vehicle information.

183. The system of any one of embodiments 178 to 182, wherein the vehicle information comprises vehicle awareness data.

184. A system for improving control of a vehicle, the system comprising:
a speaker capable of communicating audio signals to a person; and
a controller that controls the speaker to communicate: a first tone when the controller determines that the vehicle is a first distance from a marker, a second tone when the controller determines that the vehicle is a second distance from the marker, and a third tone when the controller determines that the vehicle is a first third from the marker.

185. The system of embodiment 184, wherein the second tone has a longer duration than the second tone.

186. The system of embodiment 184 or 185, wherein the third tone is louder than the second tone.

187. The system of any one of embodiments 184 to 186, wherein the second distance from the marker is less than the first distance from the marker.

188. The system of any one of embodiments 184 to 187, wherein the third distance from the marker is zero.

189. The system of any one of embodiments 184 to 188, wherein the timing of the third tone corresponds to the desired timing for the driver to apply the brakes.

190. The system of any one of embodiments 184 to 189, wherein the marker is a braking marker.

191. The system of any one of embodiments 184 to 190, wherein the position of the marker is accessed by the controller.

192. A system for improving control of a vehicle, the system comprising:
a speaker capable of communicating audio signals to a person;
a haptic system capable of communicating haptic signals to the person; and
a controller that controls the speaker and the haptic system to communicate performance-related information to the person;
wherein the controller generates a message with a haptic signal and an audio signal, the message communicating performance related information to a person.

193. The system of embodiment 192, wherein the haptic signals and the audio signals are delivered simultaneously to the person.

194. The system of embodiment 192 or 193, wherein the haptic signals and the audio signals convey the same information to the person.

195. The system of any one of embodiments 192 to 194, wherein the haptic signals and the audio signals convey different information.

196. A system for improving racing vehicle driver performance, the system comprising:
a speaker configured to communicate audio messages to a driver of a racing vehicle; and
a controller configured to cause the speaker to communicate performance-related information to the driver during movement of the racing vehicle, the controller configured to receive vehicle information, including vehicle information collected by vehicle sensors of the racing vehicle, and configured to use the vehicle information to select the audio messages to convey to the driver, the audio messages comprising (1) cue messages regarding actions to be performed by the driver, and (2) feedback messages that convey in real time to the driver whether actions are being performed properly by the driver,
wherein the cue messages comprise a series of tones indicative the actions to be performed by the driver,
wherein the feedback messages comprise a continuous chord indicative in real time of the actions being performed by the driver, and
wherein the controller is configured to cause the speaker to communicate the cue messages and the feedback messages simultaneously such that the cue messages overlap with the feedback messages with the cue messages being played intermittently while the feedback messages are played continuously.

197. The system of embodiment 196, wherein cue messages comprise a first series of tones and a second series of tones, wherein the first series of tones differs from the second series of tones by tempo or tone.

198. The system of embodiment 197, wherein the first series of tones are indicative of a marker or an object on a racetrack being a predetermined distance away from the racing vehicle on the racetrack, and wherein the second series of tones are indicative of the racing vehicle being at the marker or the object on the racetrack.

199. The system of embodiment 198, wherein the marker is a braking marker on the racetrack corresponding to where the actions to be performed by the driver comprise a braking action to decelerate the racing vehicle.

200. The system of embodiment 198 or 199, wherein the controller is configured cause the speaker to stop the cue messages after the racing vehicle passes the marker or the object on the racetrack.

201. The system of embodiment 200, wherein the controller is configured to cause the speaker to communicate the feedback messages to correspond to restoring weight distribution on wheels of the racing vehicle without playing cue messages after the racing vehicle passes the marker or the object on the racetrack.

202. The system of embodiment 201, wherein the controller is configured to cause the speaker to communicate the feedback messages to correspond to restoring weight distribution on the wheels of the racing vehicle when the racing vehicle is exiting from a corner of the racetrack.

203. The system of embodiment 202, wherein the controller is configured to cause the speaker to communicate the feedback messages to be disharmonious or discordant when the weight distribution on the wheels of the racing vehicle is not within a predetermined threshold, and wherein the controller is configured to cause the speaker to communicate the feedback messages to be harmonious when the weight distribution on the wheels of the racing vehicle is within the predetermined threshold.

204. The system of any one of embodiments 196 to 203, wherein the controller is configured to cause the speaker to communicate the continuous chord to be harmonious when the actions are within a desired operating threshold for operating the racing vehicle.

205. The system of embodiment 204, wherein the controller is configured to cause the speaker to communicate the continuous chord is disharmonious or discordant when the actions are not within the desired operating threshold for operating the racing vehicle.

206. The system of embodiment 205, wherein the continuous chord is continuously varied between harmonious and disharmonious based on an extent of the actions being within or not within the desired operating threshold, wherein the greater the extent of the actions being within the desired operating threshold, the relatively more harmonious the continuous chord is, and wherein the greater the extent of the actions being not within the desired operating threshold, the relatively more disharmonious the continuous chord is.

207. The system of any one of embodiments 196 to 206, wherein the controller is configured to prioritize communication of the cue messages over the feedback messages.

208. The system of any one of embodiments 196 to 207, further comprising at least two speakers comprising the speaker, wherein at least one of the cue messages or the feedback messages comprises directional sound indicative of a direction of a marker or an object on a racetrack.

209. The system of any one of embodiments 196 to 208, further comprising a tactor configured to communicate haptic messages to the driver of the racing vehicle, wherein the controller is configured to cause the tactor to generate a series of vibrations corresponding to the series of tones to emphasize the cue messages to the driver.

210. The system of any one of embodiments 196 to 209, further comprising a tactor configured to communicate haptic messages to the driver of the racing vehicle, wherein the controller is configured to cause the tactor to generate vibrations communicating a directional cue with respect to a racetrack.

211. The system of embodiment 210, wherein the controller is configured to cause the tactor to generate the haptic messages to communicate information to the driver corresponding to information communicated by at least one of the cue messages or the feedback messages.

212. The system of any one of embodiments 196 to 211, further comprising a tactor configured to communicate haptic messages to the driver of the racing vehicle, wherein the controller is configured to cause the tactor to generate vibrations communicating information to the driver different from information communicated by the cue messages and the feedback messages.

213. The system of any one of embodiments 196 to 212, wherein the system is part of a simulator and the racing vehicle is simulated.

214. The system of any one of embodiments 196 to 213, wherein the controller is configured to store the conveyed audio messages in a memory and to convey the stored audio messages to a person after the actions are performed by the driver.

215. A method for improving racing vehicle driver performance, the method comprising:
detecting a driver-initiated event performed during driving of a racing vehicle, said detecting performed by a computing device that monitors real time vehicle information associated with the racing vehicle;
determining a desired driver action of the driver-initiated event based at least partly on collected sensor data;
comparing actual driver action of the driver-initiated event to the desired driver action; and
delivering to a driver an audio message that is dependent on a result of the comparison,
wherein the audio message is harmonious when the actual driver action is within a predetermined threshold corresponding to the desired driver action, and
wherein the audio message is disharmonious or discordant when the actual driver action is not within the predetermined threshold corresponding to the desired driver action.

216. The method of embodiment 215, wherein the comparing the actual driver action to the desired driver action is repeatedly or continuously performed and further comprising continuously varying the audio message between harmonious and disharmonious based on the repeated or continuous comparison, wherein the audio message is continuously varied to be relatively more harmonious the relatively closer the actual driver action is to the predetermined threshold and to be relatively less harmonious the relatively less close the actual driver action is to the predetermined threshold.

217. The method of embodiment 215 or 216, further comprising delivering to the driver an other audio message, the other audio message comprising a series of tones indicative of an upcoming desired driver action to be performed.

218. The method of embodiment 217, wherein the other audio message is delivered simultaneously to driver as the audio message is being delivered to the driver such that the other audio message overlaps with the audio message during delivery.

219. The method of embodiment 217 or 218, wherein the other audio message comprises a first series of tones and a second series of tones, wherein the first series of tones differs from the second series of tones by tempo or tone.

220. The method of embodiment 219, further comprising delivering the first series of tones to indicate a marker or an object on a racetrack being a predetermined distance away from the racing vehicle on the racetrack and delivering the second series of tones to indicate the racing vehicle being at the marker or the object on the racetrack.

221. The method of embodiment 220, wherein the marker is a braking marker on the racetrack corresponding to where the upcoming desired driver action to be performed comprises a braking action to decelerate the racing vehicle as.

222. The method of embodiment 220 or 221, further comprising stopping delivery of the other audio message after the racing vehicle passes the marker on the racetrack.

223. The method of any one of embodiments 217 to 222, further comprising prioritizing communicating of the other audio message over the audio message.

224. The method of any one of embodiments 215 to 223, wherein the audio message corresponds to restoring weight distribution on wheels of the racing vehicle.

225. The method of embodiment 224, wherein the audio message corresponds to restoring weight distribution on the wheels of the racing vehicle when the racing vehicle is exiting from a corner of a racetrack.

226. The method of embodiment 224 or 225, wherein the audio message is disharmonious or discordant when the weight distribution on the wheels of the racing vehicle is not within a predetermined weight distribution threshold, and wherein the audio message is harmonious when the weight distribution on the wheels of the racing vehicle is within the predetermined weight distribution threshold.

227. The method of any one of embodiments 215 to 226, further comprising delivering to the driver a haptic message that is dependent on the result of the comparison.

228. The method of embodiment 227, wherein the haptic message comprises a directional cue indicative of a direction of a marker or an object on a racetrack.

229. The method of embodiment 227 or 228, wherein the haptic message is indicative of information corresponding to information conveyed by the audio message.

230. The method of any one of embodiments 227 to 229, wherein the haptic message is indicative of information different from information conveyed by the audio message.

231. The method of any one of embodiments 215 to 230, further comprising simulating the racing vehicle in a simulator.

232. The method of any one of embodiments 215 to 231, further comprising storing the delivered audio message in a memory and conveying the stored audio message to a person after the actual driver action is performed by the driver.

233. The method of any one of embodiments 215 to 232, wherein the audio message is communicated to the driver substantially in real time.

234. A system for improving vehicle driver performance, the system comprising:
a speaker configured to communicate audio messages to a driver of a vehicle;
a memory configured to store specific computer-executable instructions; and
a hardware processor in communication with the memory and configured to execute the specific computer-executable instructions to at least:
   receive vehicle information, including vehicle information collected by vehicle sensors of the vehicle;
   cause the speaker to communicate performance-related information to the driver during movement of the vehicle;
   select an audio message to convey to the driver based on the vehicle information, the audio message conveying to the driver whether an action is being performed as desired by the driver; and
   vary the audio message between harmonious and disharmonious dependent on whether the action is being performed as desired by the driver based on a predetermined performance threshold or range for operating the vehicle.

235. The system of embodiment 234, wherein the hardware processor is configured to execute further computer-executable instructions to continuously vary the audio message between harmonious and disharmonious dependent on an extent of whether the action is being performed as desired by the driver based on the predetermined performance threshold or range.

236. The system of embodiment 234 or 235, wherein the hardware processor is configured to execute further computer-executable instructions to select an other audio message to convey to the driver based on environmental information of the vehicle, the other audio message conveying to the driver an upcoming action to be performed by the driver.

237. The system of embodiment 236, wherein the hardware processor is configured to execute further computer-executable instructions to cause the speaker to communicate the audio message and the other audio message simultaneously such that the other audio message overlaps with the audio message.

238. The system of embodiment 237, wherein the hardware processor is configured to execute further computer-executable instructions to cause the speaker to communicate the audio message continuously and the other audio message intermittently.

239. The system of any one of embodiments 236 to 238, wherein the other audio message comprises a series of tones indicative of the upcoming action to be performed by the driver.

240. The system of embodiment 239, further comprising a tactor configured to communicate haptic messages to the driver of the vehicle, wherein the hardware processor is configured to execute further computer-executable instructions to cause the tactor to generate a series of vibrations corresponding to the series of tones to emphasize the other audio message to the driver.

241. The system of any one of embodiments 236 to 240, wherein the other audio message comprises a first series of tones and a second series of tones, wherein the first series of tones differs from the second series of tones by tempo or tone.

242. The system of embodiment 241, wherein the first series of tones are indicative of a marker or an object on a road being a predetermined distance away from the vehicle on the road, and wherein the second series of tones are indicative of the vehicle being at the marker or the object on the road.

243. The system of embodiment 242, wherein the marker is a braking marker on the road corresponding to where the upcoming action to be performed by the driver comprises a braking action to decelerate the vehicle.

244. The system of embodiment 242 or 243, wherein the hardware processor is configured to execute further computer-executable instructions to cause the speaker to stop the other audio message after the vehicle passes the marker on the road.

245. The system of embodiment 244, wherein the hardware processor is configured to execute further computer-executable instructions to cause the speaker to communicate the audio message to correspond to restoring weight distribution on wheels of the vehicle without playing the other audio message after the vehicle passes the marker on the road.

246. The system of any one of embodiments 236 to 245, wherein the hardware processor is configured to execute further computer-executable instructions to prioritize communication of the other audio message over the audio message.

247. The system of any one of embodiments 236 to 246, wherein the environmental information comprises data received by the hardware processor from a data source external to the vehicle.

248. The system of any one of embodiments 234 to 247, wherein the hardware processor is configured to execute further computer-executable instructions to cause the speaker to communicate the audio message to correspond to restoring weight distribution on wheels of the vehicle when the vehicle is exiting from a corner of a road.

249. The system of any one of embodiments 234 to 248, wherein the hardware processor is configured to execute further computer-executable instructions to cause the speaker to communicate the audio message to correspond to restoring weight distribution on wheels of the vehicle when the action is a braking action.

250. The system of embodiment 248 or 249, wherein the hardware processor is configured to execute further computer-executable instructions to cause the speaker to communicate the audio message to be disharmonious or discordant when the weight distribution on the wheels of the vehicle is not within a predetermined range or within a predetermined profile, and wherein the hardware processor is configured to execute further computer-executable instructions to cause the speaker to communicate the audio message to be harmonious when the weight distribution on the wheels of the vehicle is within the predetermined range or within the predetermined profile, wherein the predetermined profile comprises a desired rate of change of the weight distribution on the wheels of the vehicle.

251. The system of any one of embodiments 234 to 250, wherein the hardware processor is configured to execute further computer-executable instructions to cause the speaker to communicate the audio message to be harmonious when the action is within the predetermined performance threshold or range for operating the vehicle.

252. The system of any one of embodiments 234 to 251, wherein the hardware processor is configured to execute further computer-executable instructions to cause the speaker to communicate the audio message to be disharmonious or discordant when the action is not within the predetermined performance threshold or range for operating the vehicle.

253. The system of embodiment 252, wherein the audio message is continuously varied between harmonious and disharmonious based on an extent of the action being within or not within the predetermined performance threshold or range, wherein the greater the extent of the action being within the predetermined performance threshold or range, the relatively more harmonious the audio message is, and wherein the greater the extent of the action being not within the predetermined performance threshold or range, the relatively more disharmonious the audio message is.

254. The system of any one of embodiments 234 to 253, further comprising at least two speakers comprising the speaker, wherein the hardware processor is configured to execute further computer-executable instructions to cause the at least two speakers to communicate the audio message to convey directional information indicative of a direction of a marker or an object on a road.

255. The system of any one of embodiments 234 to 254, further comprising a tactor configured to communicate a haptic message to the driver of the vehicle, wherein the hardware processor is configured to execute further computer-executable instructions to cause the tactor to generate vibrations communicating a directional cue with respect to a road.

256. The system of embodiment 255, wherein the hardware processor is configured to execute further computer-executable instructions to cause the tactor to generate the haptic message to communicate information to the driver corresponding to information communicated by the audio message.

257. The system of embodiment 255 or 256, wherein the hardware processor is configured to execute further computer-executable instructions to cause the tactor to generate the haptic message to communicate information to the driver different from the information communicated by the audio message.

258. The system of any one of embodiments 234 to 257, wherein the system is part of a simulator and the vehicle is simulated.

259. The system of any one of embodiments 234 to 258, wherein the hardware processor is configured to execute further computer-executable instructions to store the conveyed audio message in the memory and to convey the stored audio message to a person after the action is performed by the driver.

260. The system of any one of embodiments 234 to 259, wherein the audio message comprises a first instrumental sound and a second instrumental sound, the first instrumental sound corresponding to a first operating parameter, and the second instrumental sound corresponding to a second operating parameter.

261. The system of embodiment 260, wherein the first operating parameter corresponds to a first driver action for operating the vehicle.

262. The system of embodiment 260 or 261, wherein the second operating parameter corresponds to a second driver action for operating the vehicle.

263. The system of embodiment 260 or 261, wherein the second operating parameter corresponds to environmental awareness data of the vehicle.

264. The system of any one of embodiments 260 to 263, wherein the hardware processor is configured to execute further computer-executable instructions to cause the speaker to play the first and second instrumental sounds simultaneously such that the first and second instrumental sounds overlap.

265. The system of embodiment 264, wherein the hardware processor is configured to execute further computer-executable instructions to prioritize the first instrumental sound or the second instrumental sound and to modulate the prioritized first or second instrumental sound.

266. The system of embodiment 265, wherein modulating the prioritized first or second instrumental sound comprises playing the prioritized first or second instrumental sound louder relative to the non-prioritized first or second instrumental sound.

267. The system of any one of embodiments 234 to 266, wherein the vehicle is a racing vehicle.

268. The system of any one of embodiments 234 to 267, wherein the vehicle is a racecar.

269. The system or method of any one of embodiments 1 to 268, wherein action or timing comprises driver input to perform desired or predetermined energy harvesting and/or energy deployment of the vehicle while operating the vehicle.

Methods of using the foregoing system(s) (including device(s), apparatus(es), assembly(ies), structure(s), and/or the like) disclosed herein are included; the methods of use can include using or assembling any one or more of the features disclosed herein to achieve functions and/or features of the system(s) as discussed in this disclosure. Methods of manufacturing the foregoing system(s) disclosed herein are included; the methods of manufacture can include providing, making, connecting, assembling, and/or installing any one or more of the features of the system(s) disclosed herein to achieve functions and/or features of the system(s) as discussed in this disclosure.

### Terminology

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

Depending on the embodiment, certain acts, events, or functions of any of the algorithms described herein can be performed in a different sequence, can be added, merged, or left out altogether (e.g., not all described acts or events are necessary for the practice of the algorithms). Moreover, in certain embodiments, acts or events can be performed concurrently, e.g., through multi-threaded processing, interrupt processing, or multiple processors or processor cores or on other parallel architectures, rather than sequentially.

Although these inventions have been disclosed in the context of certain preferred embodiments and examples, it will be understood by those skilled in the art that the present inventions extend beyond the specifically disclosed embodiments to other alternative embodiments and/or uses of the inventions and obvious modifications and equivalents thereof. In addition, while several variations of the inventions have been shown and described in detail, other modifications, which are within the scope of these inventions, will be readily apparent to those of skill in the art based upon this disclosure. It is also contemplated that various combination or sub-combinations of the specific features and aspects of the embodiments may be made and still fall within the scope of the inventions. It should be understood that various features and aspects of the disclosed embodiments can be combined with or substituted for one another in order to form varying modes of the disclosed inventions. Thus, it is intended that the scope of at least some of the present inventions herein disclosed should not be limited by the particular disclosed embodiments described above.

1. A system for improving racing vehicle driver performance, the system comprising:
a speaker configured to communicate audio messages to a driver of a racing vehicle; and
a controller configured to cause the speaker to communicate performance-related information to the driver during movement of the racing vehicle, the controller configured to receive vehicle information, including vehicle information collected by vehicle sensors of the racing vehicle, and configured to use the vehicle information to select the audio messages to convey to the driver, the audio messages comprising (1) cue messages regarding actions to be performed by the driver, and (2) feedback messages that convey in real time to the driver whether actions are being performed properly by the driver,
wherein the cue messages comprise a series of tones indicative the actions to be performed by the driver,
wherein the feedback messages comprise a continuous chord indicative in real time of the actions being performed by the driver, and
wherein the controller is configured to cause the speaker to communicate the cue messages and the feedback messages simultaneously such that the cue messages overlap with the feedback messages with the cue messages being played intermittently while the feedback messages are played continuously.

2. The system of claim 1, wherein cue messages comprise a first series of tones and a second series of tones, wherein the first series of tones differs from the second series of tones by tempo or tone.

3. The system of claim 2, wherein the first series of tones are indicative of a marker or an object on a racetrack being a predetermined distance away from the racing vehicle on the racetrack, and wherein the second series of tones are indicative of the racing vehicle being at the marker or the object on the racetrack.

4. (canceled)

5. (canceled)

6. (canceled)

7. (canceled)

8. (canceled)

9. The system of claim 1, wherein the controller is configured to cause the speaker to communicate the continuous chord to be harmonious when the actions are within a desired operating threshold for operating the racing vehicle.

10. The system of claim 9, wherein the controller is configured to cause the speaker to communicate the continuous chord is disharmonious or discordant when the actions are not within the desired operating threshold for operating the racing vehicle.

11. The system of claim 10, wherein the continuous chord is continuously varied between harmonious and disharmonious based on an extent of the actions being within or not within the desired operating threshold, wherein the greater the extent of the actions being within the desired operating threshold, the relatively more harmonious the continuous chord is, and wherein the greater the extent of the actions being not within the desired operating threshold, the relatively more disharmonious the continuous chord is.

12. (canceled)

13. (canceled)

14. (canceled)

15. The system of claim 1, further comprising a tactor configured to communicate haptic messages to the driver of the racing vehicle, wherein the controller is configured to cause the tactor to generate vibrations communicating a directional cue with respect to a racetrack.

16. (canceled)

17. (canceled)

18. (canceled)

19. (canceled)

20. A method for improving racing vehicle driver performance, the method comprising:
detecting a driver-initiated event performed during driving of a racing vehicle, said detecting performed by a computing device that monitors real time vehicle information associated with the racing vehicle;
determining a desired driver action of the driver-initiated event based at least partly on collected sensor data;
comparing actual driver action of the driver-initiated event to the desired driver action; and
delivering to a driver an audio message that is dependent on a result of the comparison,
wherein the audio message is harmonious when the actual driver action is within a predetermined threshold corresponding to the desired driver action, and
wherein the audio message is disharmonious or discordant when the actual driver action is not within the predetermined threshold corresponding to the desired driver action.

21. The method of claim 20, wherein the comparing the actual driver action to the desired driver action is repeatedly or continuously performed and further comprising continuously varying the audio message between harmonious and disharmonious based on the repeated or continuous comparison, wherein the audio message is continuously varied to be relatively more harmonious the relatively closer the actual driver action is to the predetermined threshold and to be relatively less harmonious the relatively less close the actual driver action is to the predetermined threshold.

22. The method of claim 20 or 21, further comprising delivering to the driver an other audio message, the other audio message comprising a series of tones indicative of an upcoming desired driver action to be performed.

23. The method of claim 22, wherein the other audio message is delivered simultaneously to driver as the audio message is being delivered to the driver such that the other audio message overlaps with the audio message during delivery.

24. The method of claim 22 or 23, wherein the other audio message comprises a first series of tones and a second series of tones, wherein the first series of tones differs from the second series of tones by tempo or tone.

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. A system for improving vehicle driver performance, the system comprising:
a speaker configured to communicate audio messages to a driver of a vehicle;
a memory configured to store specific computer-executable instructions; and
a hardware processor in communication with the memory and configured to execute the specific computer-executable instructions to at least:
receive vehicle information, including vehicle information collected by vehicle sensors of the vehicle;
cause the speaker to communicate performance-related information to the driver during movement of the vehicle;
select an audio message to convey to the driver based on the vehicle information, the audio message conveying to the driver whether an action is being performed as desired by the driver; and
vary the audio message between harmonious and disharmonious dependent on whether the action is being performed as desired by the driver based on a predetermined performance threshold or range for operating the vehicle.

40. The system of claim 39, wherein the hardware processor is configured to execute further computer-executable instructions to continuously vary the audio message between harmonious and disharmonious dependent on an extent of whether the action is being performed as desired by the driver based on the predetermined performance threshold or range.

41. The system of claim 39, wherein the hardware processor is configured to execute further computer-executable instructions to select an other audio message to convey to the driver based on environmental information of the vehicle, the other audio message conveying to the driver an upcoming action to be performed by the driver.

42. The system of claim 41, wherein the hardware processor is configured to execute further computer-executable instructions to cause the speaker to communicate the audio message and the other audio message simultaneously such that the other audio message overlaps with the audio message.

43. The system of claim 42, wherein the hardware processor is configured to execute further computer-executable instructions to cause the speaker to communicate the audio message continuously and the other audio message intermittently.

44. The system of claim 41, wherein the other audio message comprises a series of tones indicative of the upcoming action to be performed by the driver.

45. The system of claim 44, further comprising a tactor configured to communicate haptic messages to the driver of the vehicle, wherein the hardware processor is configured to execute further computer-executable instructions to cause the tactor to generate a series of vibrations corresponding to the series of tones to emphasize the other audio message to the driver.

46. The system of claim 41, wherein the other audio message comprises a first series of tones and a second series of tones, wherein the first series of tones differs from the second series of tones by tempo or tone.

47. The system of claim 46, wherein the first series of tones are indicative of a marker or an object on a road being a predetermined distance away from the vehicle on the road, and wherein the second series of tones are indicative of the vehicle being at the marker or the object on the road.

48. The system of claim 47, wherein the marker is a braking marker on the road corresponding to where the upcoming action to be performed by the driver comprises a braking action to decelerate the vehicle.

49. (canceled)

50. (canceled)

51. (canceled)

52. (canceled)

53. The system of claim 39, wherein the hardware processor is configured to execute further computer-executable instructions to cause the speaker to communicate the audio message to correspond to restoring weight distribution on wheels of the vehicle when the vehicle is exiting from a corner of a road.

54. The system of claim 39, wherein the hardware processor is configured to execute further computer-executable instructions to cause the speaker to communicate the audio message to correspond to restoring weight distribution on wheels of the vehicle when the action is a braking action.

55. The system of claim 53, wherein the hardware processor is configured to execute further computer-executable instructions to cause the speaker to communicate the audio message to be disharmonious or discordant when the weight distribution on the wheels of the vehicle is not within a predetermined range or within a predetermined profile, and wherein the hardware processor is configured to execute further computer-executable instructions to cause the speaker to communicate the audio message to be harmonious when the weight distribution on the wheels of the vehicle is within the predetermined range or within the predetermined profile, wherein the predetermined profile comprises a desired rate of change of the weight distribution on the wheels of the vehicle.

56. The system of claim 39, wherein the hardware processor is configured to execute further computer-executable instructions to cause the speaker to communicate the audio message to be harmonious when the action is within the predetermined performance threshold or range for operating the vehicle.

57. The system of claim 39, wherein the hardware processor is configured to execute further computer-executable instructions to cause the speaker to communicate the audio message to be disharmonious or discordant when the action is not within the predetermined performance threshold or range for operating the vehicle.

58. The system of claim 57, wherein the audio message is continuously varied between harmonious and disharmonious based on an extent of the action being within or not within the predetermined performance threshold or range, wherein the greater the extent of the action being within the predetermined performance threshold or range, the relatively more harmonious the audio message is, and wherein the greater the extent of the action being not within the predetermined performance threshold or range, the relatively more disharmonious the audio message is.

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)
